(19) United States
(12) Patent Application Publication
(10) Pub. No.: US 2026/0076674 A1
(43) Pub. Date: Mar. 19, 2026

(54) DEVICES AND METHODS FOR REPAIRING AND SEALING TISSUE FENESTRATIONS DURING OPEN AND MINIMALLY INVASIVE SURGICAL PROCEDURES

(71) Applicant: PatchClamp MedTech, Inc., Newark, DE (US)

(72) Inventors: Marc Robert Mayberg, Seattle, WA (US); Adam Lee Kosky, Palm Desert, CA (US); David Andrew Desmarais, Seattle, WA (US)

(73) Assignee: PatchClamp MedTech, Inc., Newark, DE (US)

(21) Appl. No.: 19/325,842

(22) Filed: Sep. 11, 2025

Related U.S. Application Data
(60) Provisional application No. 63/695,303, filed on Sep. 16, 2024.
(60) Provisional application No. 63/878,084, filed on Sep. 8, 2025.
(60) Provisional application No. 63/878,848, filed on Sep. 9, 2025.

(51) Int. Cl. A61B 17/08; A61B 17/00; A61B 17/10

(52) U.S. Cl. CPC A61B 17/083; A61B 17/00234; A61B 17/10; A61B 2017/00367; A61B 2017/00867; A61B 2017/00938

(57) ABSTRACT

Provided are tissue repair and sealing devices, and methods for the use of tissue repair and sealing devices, for use in both minimally invasive surgical (MIS) procedures and open, non-MIS procedures to rapidly repair tissue fenestrations and create a pressure-resistant, watertight seal in a tissue barrier. Tissue repair and sealing devices disclosed herein comprise an integrated graft and deployable clasp assembly and an applicator assembly having a graft-clasp lock cylinder that is slidably connected to a folded, deployable clasp. The applicator assembly places a graft on a tissue inner surface and a deployable clasp on a tissue outer surface to secure the graft to the tissue inner surface to, thereby, repair a tissue fenestration and create a watertight barrier.

### CROSS-REFERENCE TO RELATED APPLICATIONS

This U.S. non-provisional patent application was filed on Sep. 11, 2025 as U.S. patent application Ser. No. 19/325,842 and claims the benefit of U.S. Provisional Patent Application No. 63/878,848, which was filed on Sep. 9, 2025, U.S. Provisional Patent Application No. 63/878,084, which was filed on Sep. 8, 2025, and U.S. Provisional Patent Application No. 63/695,303, which was filed on Sep. 16, 2024. The contents of U.S. Provisional Patent Application Nos. 63/878,848, 63/878,084, and 63/695,303 are incorporated herein by reference in their entirety.

### BACKGROUND OF THE DISCLOSURE

### Technical Field

The present disclosure relates, generally, to the field of medicine, in particular to surgical procedures, including both minimally invasive (MIS) and open (non-MIS) surgical procedures. Disclosed herein are devices, and methods for their use, for repairing and sealing tissue fenestrations, such as those that occur during MIS and non-MIS surgical procedures or that are due to other processes (e.g., congenital, infectious, or neoplastic diseases). Tissue repair and sealing devices comprise a graft-clasp and applicator assembly that permits the positioning of a graft on an inner tissue surface and securing that graft with a clasp that is deployed on an outer tissue surface and locked into a coupler to repair a fenestrated tissue and create a pressure-resistant, watertight seal.

### Background and Related Art

Advances in endoscopic, robotic, and microsurgical technology have permitted the rapid advancement of minimally invasive surgical (MIS) procedures whereby a surgical site is accessed through a small incision. For example, MIS procedures are used to access working spaces within a body cavity or body space (e.g., an abdominal cavity, a cranial sinus, an intracranial space, or a peraspinal tissue) or a luminal pathway (e.g., a cardiovascular system, a gastrointestinal system, a cranial or spinal cerebrospinal fluid pathway, or an organ, such as a uterus, a bladder, or a kidney).

Several factors common to MIS procedures, including limited working space, restricted surgical access, poor visualization, and the friable nature of certain tissues, make it difficult to repair and seal cuts, tears, or openings in tissues that are beneath the skin (collectively tissue fenestrations). Failure to rapidly repair a tissue fenestration and create a pressure resistant watertight seal results in the leakage of body fluids through the fenestrated tissue, which inhibits tissue healing, promotes infection, and leads to substantial post-surgical morbidity.

Dural perforations (durotomies), which can (1) occur spontaneously (e.g., congenital defects); (2) arise secondarily to tumor formation, trauma, or infections; or (3) result intentionally through a planned incision/puncture or unintentionally during a surgical procedure. Durotomies range in size from millimeters (e.g., lacerations) to several square centimeters (e.g., resection of dura for tumors).

Incidental spinal durotomy is relatively common and particularly significant due to the sheer volume and cost of spinal surgeries (in the U.S the estimated annual procedural volume for spinal surgeries is over 1.6 million) and because cerebrospinal fluid (CSF), which fills a space between the outerdural layer and adjacent neural or vascular structures, is under pulsatile pressure in the range of 8 to 15 cm within the cranium, fenestrated dural tissue is particularly challenging to repair. See, McDermott and Liang, HCUP Statistical Brief #281 (2018) and Guerin et al., Injury 43(4):397-401 (2012).

Incomplete dural seals in spinal and cranial surgeries represent a substantial challenge, leading to increased mortality, infection rates, lengths of hospital stay, and healthcare costs. This is especially prevalent in minimally invasive surgery (MIS) where access and visibility are limited. The intrinsic dural pressure, which is higher than the external atmospheric pressure, contributes to continued leaking of CSF and failure of dura to heal spontaneously. Postoperative cerebrospinal fluid (CSF) leaks due to incompletely sealed dural perforations (durotomies) are relatively common for spinal surgery (1-19% in Minimally Invasive Surgery [MIS] and 1-15% in open cases), Ghobrial et al., Neurosurg. Focus 39(4) (2015) and Prevedello et al., Neurosurg. Focus 58(2):E1 (2025) and for complex MIS endonasal cranial surgery (10-33%). White et al., Barrow Q 18(3) (2002).

Existing devices for attaching grafts to the outer surface of tissue fenestrations have limited utility in MIS procedures. Devices that are known in the art are difficult to manipulate and typically require additional procedures (e.g., harvesting a tissue for buttressing and placing drains to reduce pressure gradients). Moreover, tissue grafts attached to an outer tissue surface, are prone to failure and are particularly susceptible to pressure differentials between the inside and outside of a fenestrated tissue (e.g., a fenestrated blood vessel, dura mater, or gastrointestinal wall tissue). Because grafts positioned on an outer tissue surface often fail to repair tissue fenestrations and create watertight seals, fluids leak from a higher-pressure tissue interior (e.g., blood, cerebrospinal fluid, or gastrointestinal contents).

Currently available techniques for closing tissue fenestrations during MIS procedures (which include (a) suturing or stapling, (b) tissue adhesive, and (c) tissue grafts), are time-consuming and technically difficult in the limited space and restricted access that is characteristic of MIS procedures. They often are ineffective in providing an immediate pressure-resistant watertight closure and are associated with unacceptable postoperative failure rates leading to significant complications and substantial increase in cost of care. Choi et al., World Neurosurg. 149:140-147 (2021). The development of innovative endoscopic MIS approaches to the brain have been limited in part by the difficulty in re-establishing integrity of the dura at the skull base. Mayberg, J. Neurosurg. 114(6):1541-1542 (2011) and Jaffray, Archives of Disease in Childhood 90:537-542 (2005). And metallic implants (staples) can interfere with subsequent magnetic resonance imaging.

Tissue patches, which include (1) autologous patches (e.g., fascia and fat), (2) heterologous (e.g., bovine or porcine), or synthetic (e.g., collagen matrix), require the use of sutures or a tissue adhesive to hold the patch in place. Moreover, tissue patches are susceptible to infection and tissue rejection. Pedicle graft overlays to facilitate healing require an additional procedure with a glue or buttress to ensure adherence, do not provide immediate watertight closure, and are associated with elevated surgical morbidity.

Absorbable and non-absorbable tissue adhesives, such as fibrin glue and polyglycol gel, also have limited utility in the rapid repair of tissue fenestrations and creation of pressure-resistant, watertight seals, and pose substantial technical challenges that can contribute to poor surgical results, namely: (1) the required mixing and applying of a rapidly-curing, two-component adhesive is difficult to perform in a small space; (2) buttressing of a graft with another tissue (e.g., fat) is often necessary; and (3) the bonding strength of tissue adhesives can be inadequate for the creation of a pressure-resistant, watertight seal.

Despite the availability of existing technologies for closing tissue fenestrations during surgical procedures, there exists a significant unmet need for improved technologies for repairing and sealing tissues during both open and MIS procedures, including devices and methods that permit the rapid repair of tissue fenestrations and the reliable creation of pressure-resistant, watertight seals. The present disclosure fulfills these needs and provides further related advantages over existing technologies for use in open and minimally invasive surgical (MIS) procedures.

### SUMMARY OF THE DISCLOSURE

Provided herein are tissue repair and sealing devices that exhibit unexpected and surprising advantages over devices and technologies that are currently available in the art for repairing and sealing tissue fenestrations—including those that occur during minimally invasive (MIS) and open (non-MIS) surgical procedures.

The tissue repair and sealing devices disclosed herein fulfill an unmet need in the art for surgical devices that improve the closure of tissue fenestrations. These devices require only one hand to place a graft directly against a tissue inner surface at a site of perforation and to secure that graft by locking a clasp against a tissue outer surface to create a pressure-resistant, watertight seal. Through in vitro and in vivo testing, substantial improvements in sealing time and effectiveness have been obtained as compared to traditional devices and methods known in the art. Hanks et al., J. Neurosurg Spine (submitted).

Within various embodiments, the tissue repair and sealing devices, and methods for their use, which are disclosed herein, permit the rapid and reliable repair of tissue fenestrations and creation of watertight seals that are resistant to pressure differentials such as those that occur across the inside and outside of normal tissues. Thus, these tissue repair and sealing devices provide a surgeon with ease of use and flexibility within a restricted working space with a corresponding reduction in complications related to postoperative fluid leakage.

Tissue repair and sealing devices disclosed herein comprise (1) a graft-clasp positioning mechanism that permits the positioning of a graft on a tissue inner surface and a clasp on a tissue outer surface; (2) a graft-clasp lock mechanism that locks a clasp into a graft-clasp coupler on the graft-clasp subassembly, and (3) a graft-clasp release mechanism that disconnects the graft-clasp subassembly from the applicator subassembly at the distal end of the graft positioning cylinder.

The tissue repair and sealing devices disclosed herein include assemblies comprising (1) an applicator subassembly and (2) a graft-clasp subassembly. Within certain embodiments, an applicator subassembly includes, (i) a deployable graft-clasp lock mechanism, (ii) a deployable graft-clasp release mechanism, and (iii) a deployable graft-clasp positioning mechanism. Within these and other embodiments, graft-clasp subassemblies include, in operable connection, (i) a foldable graft, (ii) a deployable clasp, and (iii) a bioabsorbable coupler that joins the graft to the clasp and, in some embodiments, provides a recess or other feature that permits the clasp to lock into the coupler to secure a graft on a tissue inner surface and, thereby, create a pressure-resistant, watertight seal.

Tissue repair and sealing devices according to these embodiments comprise first, second, and third cylinders that are arranged concentrically (i.e. nested), each cylinder having a proximal end and a distal end wherein (1) a graft-clasp lock mechanism comprises a graft-clasp lock cylinder having a first diameter, (2) a graft-clasp release mechanism comprises a graft-clasp release cylinder having a second diameter that is less than the first diameter, and (3) a graft-clasp positioning mechanism comprises a graft-clasp positioning cylinder having a third diameter that is less than the second diameter.

Within some aspects of these embodiments, deploying a graft-clasp positioning mechanism permits the sequential positioning of a graft on a tissue inner surface and a clasp on a tissue outer surface. Within other aspects of these embodiments, deploying a graft-clasp lock mechanism locks a clasp into a graft-clasp coupler. Within further aspects of these embodiments, deploying a graft-clasp release mechanism disconnects a graft-clasp subassembly from an applicator subassembly at the distal end of the graft positioning cylinder.

### BRIEF DESCRIPTION OF THE DRAWINGS

Certain aspects of the present disclosure will become more evident in reference to the drawings, below, including FIGS. 1-44, which present aspects of graft-clasp and applicator assemblies and methods for their use according to various embodiments of the tissue repair and sealing devices disclosed herein. In certain embodiments, these tissue repair and sealing devices comprise elements recited in Table 1, which defines each of the elements identified in the tissue repair and sealing devices shown in FIGS. 1-26.

TABLE 1
Tissue Repair and Sealing Device Elements

| | | |
|---|---|---|
| ELEMENT NO. | ELEMENT NAME | SOURCE AND NO. |

GRAFT-CLASP AND APPLICATOR ASSEMBLY

| | |
|---|---|
| 101 | Graft-Clasp and Applicator Assembly |

APPLICATOR SUBASSEMBLY

| | | |
|---|---|---|
| 102 | Applicator Subassembly | |
| 105 | Applicator Housing, Left | |
| 107 | Applicator Housing, Right | |
| 108 | Graft-Clasp Positioning Mechanism | |
| 109 | Graft-Clasp Positioning Main Shuttle, Top | |
| 115 | Graft-Clasp Positioning Main Shuttle, Bottom | |
| 135 | Graft-Clasp Positioning Main Shuttle, Rocker | |
| 187 | Graft-Clasp Positioning Cylinder (Ball Socket) | |
| 117 | Graft-Clasp Positioning Main Shuttle, Gear | |
| | Retainer | |
| 177 | Graft-Clasp Positioning Steel Metric Ball-Nose | 3391A652 |
| | Spring Plunger | |
| 124 | Graft-Clasp Lock Mechanism | |
| 125 | Graft-Clasp Lock Carriage, Frame Pusher | |
| 129 | Graft-Clasp Lock Frame Pusher, Trigger | |
| 137 | Graft-Clasp Lock Frame Pusher Rack | |
| 139 | Graft-Clasp Lock Frame Pusher Rack, Bottom | |
| 149 | Graft-Clasp Lock 20 Degree Pressure Angle Plastic | Lily Bearing |
| | Gear | 2662N24 |
| 169 | Graft-Clasp Lock Cylinder | 316 Stainless Steel |
| | | 1800T279 |
| 175 | Graft-Clasp Lock Compression Spring | 9657K696 |
| 126 | Graft-Clasp Release Mechanism | |
| 127 | Graft-Clasp Release Carriage Patch Lock Release | |
| 119 | Graft-Clasp Release Carriage Eject | |
| 145 | Graft-Clasp Release Eject Lever | |
| 147 | Graft-Clasp Release Patch Lock Release Gear | |
| 155 | Graft-Clasp Release Eject Rack | |
| 157 | Graft-Clasp Release Button Head Hex Drive Screw | Lily Bearing |
| | | 92095A181 |
| 159 | Graft-Clasp Release Gear Pivot Lock | |
| 165 | Graft-Clasp Release Gear Pivot Sleeve | |
| 167 | Graft-Clasp Release Low-Carbon Steel Bar | 9143K259 |
| 189 | Graft-Clasp Release Cylinder | 316 Stainless Steel |

GRAFT-CLASP SUBASSEMBLY

| | | |
|---|---|---|
| 103 | Graft-Clasp Subassembly | |
| 179 | Graft-Clasp Coupler (Post and Ball) | |
| 185 | Graft-Clasp Graft (Patch) | |
| 195 | Graft-Clasp Clasp_5B | 1.005B-2 |
| 197 | Graft-Clasp Clasp_5A | 1.005-3 |
| 199 | Graft-Clasp Clasp_5C | 1.005C-1 |
| 205 | Graft-Clasp Clasp_13 | 1.013-1 |
| 207 | Graft-Clasp Clasp_12 | 1.012-1 |
| 209 | Graft-Clasp Clasp_14 | 1.014-1 |
| 212 | Graft-Clasp Clasp Release Surface | |
| 214 | Graft-Clasp Clasp Coupler Lock | |
| 215 | Graft-Clasp Clasp Strut | |

*** Limitations may include: (1) biophysical parameters, (2) materials, (3) bioresorbability, (4) immunogenicity, and (5) drug elution matrix

FIG. 1 is a left-side perspective view of an exemplary graft-clasp and applicator assembly 101 according to embodiments of the tissue repair and sealing devices disclosed herein.

FIG. 2 is a right-side perspective view of an exemplary applicator subassembly graft-clasp positioning mechanism 108 according to embodiments of the tissue repair and sealing devices disclosed herein.

FIG. 3 is a left-side perspective view of an exemplary applicator subassembly graft-clasp lock mechanism 124 according to embodiments of the tissue repair and sealing devices disclosed herein.

FIG. 4 is a right-side perspective view of an exemplary applicator subassembly graft-clasp release mechanism 126 according to embodiments of the tissue repair and sealing devices disclosed herein.

FIG. 5 is a left-side view of an exemplary applicator subassembly 102 according to embodiments of the tissue repair and sealing devices disclosed herein.

FIG. 6 is a left-side view of an exemplary applicator subassembly 102 (shown without housing) according to embodiments of the tissue repair and sealing devices disclosed herein.

FIG. 7 is a left-side view of an exemplary graft-clasp and applicator assembly 101 (shown without housing and without shuttle top) according to embodiments of the tissue repair and sealing devices disclosed herein.

FIG. 8 is a right-side view of an exemplary applicator subassembly 102 according to embodiments of the tissue repair and sealing devices disclosed herein.

FIG. 9 is a right-side view of an exemplary applicator subassembly 102 (shown without housing) according to embodiments of the tissue repair and sealing devices disclosed herein.

FIG. 10 is a right-side view of an exemplary graft-clasp and applicator assembly 101 (shown without housing and without shuttle top) according to embodiments of the tissue repair and sealing devices disclosed herein.

FIG. 11 is a top view (shown without housing and without shuttle top) of an exemplary applicator subassembly 102 according to embodiments of the tissue repair and sealing devices disclosed herein.

FIG. 12 is a bottom view (shown without housing and without shuttle top) of an exemplary applicator subassembly 102 according to embodiments of the tissue repair and sealing devices disclosed herein.

FIG. 13 is a front view of an exemplary graft-clasp and applicator assembly 101 according to embodiments of the tissue repair and sealing devices disclosed herein.

FIG. 14 is a back view of an exemplary graft-clasp and applicator assembly 101 according to embodiments of the tissue repair and sealing devices disclosed herein.

FIG. 15 is a right-side perspective view of exemplary graft-clasp subassemblies 103 according to embodiments of the tissue repair and sealing devices disclosed herein (shown with alternative clasp configurations).

FIGS. 16A-16D are perspective side, top and bottom views of the external clasp (FIG. 16A-C) and right side schematic views (FIG. 16C-F) of the graft-clasp subassembly locking mechanism according to embodiments of the tissue repair and sealing devices disclosed herein.

FIG. 17A-D are distal end views of an exemplary graft-clasp subassembly releasably attached to the distal end of an applicator subassembly graft-clasp positioning cylinder according to embodiments of the tissue repair and sealing devices disclosed herein.

FIG. 18 is a right-side view showing the attachment of an exemplary graft-clasp subassembly 185 via coupler 179 to a ball socket cylinder 187 of an applicator subassembly according to embodiments of the tissue repair and sealing devices disclosed herein.

FIGS. 19A-19B are left side views that show the external clasp 209 mounted on the applicator subassembly 187 which is attached to the post, ball, and flange coupler 179 of the graft-clasp subassembly (and prior to locking the clasp) according to embodiments of the tissue repair and sealing devices disclosed herein.

FIGS. 20A-20C are left side views showing the enclosure of the external clasp 209 mounted on the applicator subassembly 187 by distal advancement of a sliding clasp-lock cylinder 169 according to embodiments of the tissue repair and sealing devices disclosed herein.

FIG. 21 is the left side views showing the attachment (FIG. 21A) and release (FIG. 21C) of a ball at the distal end of the applicator subassembly 187 to the coupler of the graft-clasp subassembly 179 by distal and proximal advancement of a sliding locking cylinder 18 according to embodiments of the tissue repair and sealing devices disclosed herein.

FIG. 22 shows a variety of potential external clasp radial strut 209 configurations for the graft-clasp subassembly (103) which increase the surface area of the struts against the membrane surface, thereby providing more force to stabilize the position of the graft.

FIG. 23 shows a device handpiece 102 with interchangeable graft-clasp subassembly cassettes 103 which may have grafts of different size and/or materials that can be quickly substituted on the handpiece during a surgical procedure according to embodiments of the tissue repair and sealing devices disclosed herein.

FIG. 24 illustrates how the applicator subassembly 189 of the device can be inserted through the working channel of a surgical endoscope 601 and the graft 187 visualized at the distal end for repair and sealing of a tissue fenestration during an endoscopic surgery procedure according to embodiments of the tissue repair and sealing devices disclosed herein.

FIGS. 25A-25F are perspective views showing steps in the deployment of a graft-clasp subassembly to repair a tissue fenestration according to embodiments of the tissue repair and sealing devices disclosed herein and showing that both the graft 185 and the clasp struts 209 are folded away from the proximal end of the applicator subassembly (FIG. 25B) according to embodiments of the tissue repair and sealing devices disclosed herein.

FIGS. 26A-26H are photographs showing the deployment of a graft-clasp subassembly (FIGS. 25A-25D) and the external clasp (FIGS. 25E-25H) from the clasp-lock cylinder of the device according to embodiments of the tissue repair and sealing devices disclosed herein.

FIGS. 27A-27D are photographs showing the deployment of a graft-clasp subassembly to repair and seal a fenestrated tissue in an in vitro model system according to embodiments of the tissue repair and sealing devices disclosed herein.

FIGS. 28A-28B are side (above) and top (below) views of an exemplary tissue repair and sealing device for closing dura at a burr hole showing the dural incision (FIG. 28A) which is repaired and sealed (FIG. 28B) using the device according to embodiments of the tissue repair and sealing devices disclosed herein.

FIGS. 29A-29E FIG. 29A-29K show a modification of the device to function as an external clasp attachment assembly in a stapler-like fashion by sequentially attaching several external clasps to multiple pre-implanted couplers in dural grafts for the rapid closure of long open cranial or spinal incisions according to embodiments of the tissue repair and sealing devices disclosed herein. FIGS. 29A-29E show the method by which the attachment assembly attaches an external clasp to individual pre-implanted couplers. FIGS. 29F-29K show the method by which a rectangular graft of dural substitute material placed below a long dural incision is secured with sequential application of multiple clasps.

FIGS. 30A-30F shows repair and sealing of a long open surgery craniotomy dural incision by application of multiple individual curved rectangular grafts placed sequentially end-to-end under the dura, according to embodiments of the tissue repair and sealing devices disclosed herein. In a similar fashion, FIGS. 30G-30H show a duroplasty to repair a large defect in the dura performed by securing a piece of graft material sized to the defect with multiple circumferential individual curved rectangular grafts.

FIGS. 31A-31B are perspective views showing the application of a two-component sealant after deployment of a clasp to secure a graft-clasp and seal a fenestrated tissue according to embodiments of the tissue repair and sealing devices disclosed herein.

FIGS. 32A-32C are perspective views showing test chambers for assessing the performance of tissue repair and seal devices in constrained access (MIS) lumbar surgical procedures. In FIG. 32A a pressurized cassette is inserted into a printed model of the human lumbar spine to simulate MIS spinal dural repair. FIG. 32B (side view) and FIG. 32C (top view) show a pressurized test chamber with repair and sealing device inserted through an adjustable narrow access port to access a tissue membrane for MIS simulation of tissue repair and sealing according to embodiments of the tissue repair and sealing devices disclosed herein.

FIGS. 33A-33D illustrate an embodiment of the presently disclosed tissue repair and sealing device that is configured for use via a flexible applicator tube 313 advanced over a guide wire 373 in surgical procedures (e.g., lumbar punctures and gastrostomies) to occlude a large-bore needle puncture or percutaneous ostomy site.

FIGS. 34A-34E show steps in the deployment of a graft-clasp subassembly 302 comprising a disc-shaped locking clasp 309 and compressible sponge graft 305 for use with a narrow delivery tube 315 in an endoscope or large-bore spinal needle according to various tissue repair and seal device embodiments.

FIGS. 35A-35E show an alternative configuration of the graft-clasp subassembly 303 comprised of internal and external compressible sponges for use with an endoscope according to various tissue repair and seal device embodiments.

FIGS. 36A-36D show the use of a tissue repair and sealing device comprising an intradural drug-eluting wafer for localized release and administration of a drug to the cerebrospinal fluid (CSF) according to embodiments of the tissue repair and sealing devices disclosed herein. The wafer is placed inside the dura with the applicator through a large-bore spinal needle and secured with an external clasp (FIG. 36A—coronal view; FIG. 36B—sagittal view). Drug is released from the polymer in a continuous regulated manner, where it diffuses throughout the CSF (FIG. 36C-coronal view; FIG. 36D-sagittal view.

FIGS. 37A-37D show the use of a tissue repair and sealing device comprising an extradural drug-eluting wafer for localized release and administration of a drug to an epidural space according to embodiments of the tissue repair and sealing devices disclosed herein. The wafer is placed immediately outside the dura with the applicator through a large-bore spinal needle (FIG. 37A—coronal view; FIG. 37B—sagittal view. Drug is released from the polymer in a continuous regulated manner, where it diffuses into the epidural space to provide a local effect on the nerves and spine (e.g. opioid, corticosteroid) at the level of administration (FIG. 37C—coronal view; FIG. 37D—sagittal view).

FIGS. 38A-38D are photographs demonstrating the use of a tissue repair and sealing device in a human cadaver model system for spinal endoscopy according to embodiments of the tissue repair and sealing devices disclosed herein.

FIG. 39 is a graph of strut force applied to a tissue surface by an external clasp of the device according to embodiments of the tissue repair and sealing devices disclosed herein.

FIG. 40 is a graph of lateral force stability testing of a tissue repair and sealing device graft a tissue membrane mounted in the pressure chamber over pressures from 0-35 cm H₂O.

FIG. 41 is a graph representing comparison of the amount of time required for a board-eligible neurosurgeon to repair and seal a durotomy in a constrained access pressure chamber model for the repair and sealing device vs. two contemporary repair and seal techniques.

FIG. 42 is a graph comparing measured graft bursting pressure for various durotomy repair and sealing techniques in a pressure chamber.

FIG. 43 is a graph comparing measured graft failure pressure for various repair and sealing techniques of durotomy in a pressure chamber.

FIG. 44 is a photograph demonstrating the use of a tissue repair and sealing device in a live pig laminectomy model system using a graft-clasp subassembly deployed through a 14 mm access port.

### DETAILED DESCRIPTION OF THE DISCLOSURE

The devices and methods disclosed herein fulfill an unmet need in the art by providing safe and reliable closure of dura in MIS craniospinal surgical procedures that reduces morbidity and cost of craniospinal dural repair procedures and further provides MIS approaches for CNS lesions that would otherwise require more invasive procedures such as craniotomy or laminectomy. These tissue repair and sealing devices achieve rapid attachment and stabilization of a dural graft to establish an immediate watertight barrier using a simple one-hand disposable application tool that safely inserts a graft without further tearing of the dura and that is safely removed, repositioned, or exchanged during the course of a surgical procedure.

The present disclosure provides devices and methods for their use in the surgical repair and sealing of tissue fenestrations that occur during open and minimally invasive (MIS) surgical procedures or that arise naturally through a congenital, infectious, neoplastic or other process. Tissue repair and sealing devices disclosed herein comprise a graft-clasp subassembly and an applicator subassembly that permits the positioning of a graft on an inner tissue surface and the locking of a clasp on an outer tissue surface to rapidly secure the graft, repair a fenestrated tissue, and create a pressure-resistant and watertight seal.

Thus, provided herein are tissue repair and sealing devices that comprise: (a) an applicator subassembly comprising, in operable interconnection, (i) a graft-clasp lock mechanism for deploying a graft-clasp lock cylinder, (ii) a graft-clasp release mechanism for deploying a graft-clasp release cylinder, and (iii) a graft-clasp positioning mechanism for positioning a graft-clasp subassembly and (b) a graft-clasp subassembly comprising, in operable interconnection, (i) a foldable graft, (ii) a deployable clasp, and (iii) a bioabsorbable coupler.

Tissue repair and sealing devices employ a detachable graft-clasp subassembly that includes three integrated subcomponents: (1) a graft (tissue or synthetic) that self-expands after release from an outer graft-clasp lock cylinder and is positioned via an inner graft-clasp positioning cylinder against the inner surface of the perforated tissue; (2) a bioabsorbable coupler for reversible attachment of the graft-clasp subassembly to the applicator subassembly; and (3) a bioabsorbable clasp having flexible bioabsorbable radial struts that locks into the bioabsorbable coupler to secure the graft to a tissue inner surface and the clasp to a tissue outer surface to, thereby, create a pressure-resistant, watertight seal.

A graft-clasp positioning cylinder is configured to accommodate at its distal end a folded graft-clasp subassembly and, when activated by depressing a trigger on the applicator subassembly, locks a clasp into a groove within graft-clasp coupler. The second graft-clasp lock cylinder is configured at its distal end to detach a graft-clasp subassembly from the distal end of a third graft-clasp lock cylinder. The third graft-clasp lock cylinder is configured to removably attach at its distal end to a graft-clasp subassembly for positioning a graft on an inner tissue surface and a deployable clasp on an outer tissue surface.

Deploying the first graft-clasp lock cylinder locks the clasp of the graft-clasp subassembly into a graft-clasp coupler. The graft-clasp is released by activating the second cylinder, which detaches the graft-clasp subassembly from the applicator subassembly and to, thereby, secure the graft onto an inner tissue surface to, thereby, achieve the rapid repair of a tissue fenestration and the reliable creation of a pressure-resistant, watertight seal.

The graft-clasp components snap together and if desired, may be assembled immediately prior to application, allowing interchange of grafts of differing size, shape and material. The coupling component consists of a ball-and-socket configuration between graft and applicator which allows rotation of the graft during angled approaches. The external clasp consists of multiple, radially oriented bioabsorbable clasp struts on a ring that locks onto the coupling unit to secure the graft in place by direct contact force against the outer surface of the dura. The graft and radial clasp struts are folded into the graft-clasp positioning cylinder of the device until after release and expansion of the graft followed by the outer clasp.

A surgeon passes the distal 1-2 mm of the graft-clasp positioning cylinder 187 through a tissue fenestration while simultaneously advancing the graft with the graft-clasp positioning cylinder such that the graft expands to its original flat shape (i.e., prior to folding and loading into the graft-clasp lock cylinder) in close proximity to the inside tissue surface. The expanded graft is soft and very flexible and can contact and even gently displace adjacent structures (e.g., nerve roots). Because the graft is highly flexible, it can be removed by retracting it back into the graft-clasp lock cylinder at any point without additional damage to the dura.

After expansion and positioning of the internal graft with the graft-clasp positioning cylinder (187), a second cylinder (the graft-clasp lock cylinder 169) advances the clasp along the graft-clasp positioning cylinder to release the radial clasp struts, stabilize the graft in position, and locks the clasp in a recess in the graft-clasp coupler to create a pressure-resistant, watertight seal that repairs the tissue fenestration. Actuating the graft-clasp release mechanism deploys a graft-clasp release cylinder 189 that separates the applicator subassembly 102 from the deployed graft-clasp subassembly 103. Over weeks/months, the bioabsorbable coupler and strut assembly are resorbed, and fibroblasts enter the intradural graft to establish permanent healing of the durotomy.

A graft and clasp are joined stably via a coupler to create a graft-clasp subassembly that is configured for removable attachment to/from an applicator subassembly. In use, a graft is positioned on an inner tissue surface, a clasp is positioned on an outer tissue surface and locked into a groove in the graft-clasp coupler to secure the graft against a tissue inner surface and the clasp against a tissue outer surface to create a pressure-resistant, watertight seal and, thereby, to repair a tissue fenestration.

In certain embodiments, tissue repair and sealing devices disclosed herein comprise (1) an applicator subassembly comprising, in operable connection, (i) a deployable graft-clasp lock mechanism, (ii) a deployable graft-clasp release mechanism, and (iii) a deployable graft-clasp positioning mechanism and (2) a graft-clasp subassembly comprising, in operable connection, (i) a foldable graft, (ii) a deployable clasp, and (iii) a bioabsorbable coupler.

Within some aspects of these embodiments, tissue repair and sealing devices comprise first, second, and third concentrically (i.e. nested) cylinders wherein (1) graft-clasp lock mechanisms comprising a first cylinder having a first diameter, (2) graft-clasp release mechanisms comprising a second cylinder having a second diameter that is less than the first diameter, and (3) graft-clasp positioning mechanisms comprising a third cylinder having a third diameter that is less than said second diameter and first, second, and third cylinders each have a proximal end and a distal end.

Within other aspects of these embodiments, deploying a graft-clasp positioning mechanism permits the positioning of a graft on a tissue inner surface and a clasp on a tissue outer surface tissue repair and sealing devices. Within other aspects of these embodiments, deploying a graft-clasp lock mechanism locks a clasp into a graft-clasp coupler. Within other aspects of these embodiments, deploying a graft-clasp release mechanism disconnects a graft-clasp subassembly from an applicator subassembly at the distal end of the graft positioning cylinder.

Within certain embodiments, these devices fulfill an unmet need in the art by providing safe and reliable closure of dura in MIS craniospinal surgical procedures that reduces morbidity and cost of craniospinal dural repair procedures and further provides MIS approaches for CNS lesions that would otherwise require more invasive procedures such as craniotomy or laminectomy. These tissue repair and sealing devices achieve rapid attachment and stabilization of a dural graft to establish an immediate watertight barrier using a simple one-hand disposable application tool that safely inserts a graft without further tearing of the dura and that is safely removed, repositioned, or exchanged during the course of a surgical procedure.

As demonstrated herein, the tissue repair and seal devices disclosed herein achieve rapid watertight dural closure superior to current techniques by rapidly producing a watertight closure of the dura under direct vision that can be immediately assessed, thereby saving operating room time and expense, and reducing the complications related to postoperative CSF leakage.

The devices disclosed herein can be configured for deployment through an MIS access port as small as 3 mm to greater than 14 mm, fully biodegradable and biocompatible materials, interchangeable tailored fit of graft, and unique clasp design to ensure position stability.

Grafts may include an autologous or heterologous tissue or commercially available synthetic tissue (dural) substitute that is incorporated into the device as an integrated graft-clasp unit, which can be assembled and substituted at the time of surgery for a different graft material, size and shape. Rotational coupling between applicator and graft to allow the graft to remain flat against the inner dura during angled approaches.

Within certain embodiments, tissue repair and sealing devices are low in profile to permit passage through narrow MIS ports and to facilitate better visualization of a target tissues and fenestrations. These devices fit within MIS workflow (craniospinal) and are also designed for use in open (cranial and spinal) procedures for rapid closure of dural incisions or duraplasty.

The tissue repair and sealing devices and methods disclosed herein may be employed in the direct visual, percutaneous, and/or endoscopic repair and sealing of a wide variety of human tissues. It will be understood by those of skill in the art that the tissue repair and sealing devices and methods described and exemplified herein may be adapted (without deviating from the spirit and scope of the present disclosure) to address problems specific to the nature, condition, and surgical exposure of the fenestrated tissue. Such modifications may, for example (1) vary material composition, (2) substitute graft materials (e.g., to provide biodegradability, biosorbability, and or drug elution), (3) modify sizes, shapes, and biophysical properties, (4) vary the size and/or shape of a graft-clasp unit, and (5) employ a flexible applicator for use with a guide wire (e.g., for the percutaneous or endoscopic repair and sealing of punctures or ostomies).

The present disclosure provides tissue repair and sealing devices in both MIS and non-MIS procedures to achieve the rapid repair of fenestrated tissues and the reliable creation of pressure-resistant watertight seals. The tissue repair and sealing devices disclosed herein comprise, in operable combination, (1) an applicator assembly comprising a graft-clasp lock cylinder having a proximal end and a distal end that is movably attached to an graft-clasp positioning cylinder having a proximal end and a distal end and (2) a detachable graft and clasp assembly comprising a graft subassembly and a deployable clasp and coupler subassembly that is fixedly attached to the center of the graft.

Within certain embodiments, the tissue repair and sealing devices disclosed herein comprise: (a) an applicator subassembly comprising three concentric cylinders (a first graft-clasp lock cylinder having a first diameter, a second graft-clasp lock cylinder having a second diameter, and a third graft-clasp lock cylinder having a third diameter, wherein the first diameter is greater than the second diameter and the second diameter is greater than the third diameter) and (b) a graft-clasp subassembly comprising, in operable combination, a graft, a coupler, and a clasp (wherein the first graft-clasp lock cylinder is configured to accommodate at its distal end a folded graft-clasp subassembly, the second graft-clasp lock cylinder is configured to separate said graft-clasp subassembly from said applicator subassembly, and the third graft-clasp lock cylinder is configured to removably attach at its distal end to a graft-clasp subassembly via the coupler).

Because the graft is highly flexible, it can be removed by retracting it back into the applicator at any point without additional damage to the dura. After expansion and positioning of the internal graft, a graft-clasp lock cylinder advances the external clasp along the graft-clasp positioning cylinder to sequentially release the radial clasp struts to stabilize the graft. Prior to locking, the struts apply only minimal force against the tissue, which allows the surgeon to adjust the position of the graft and confirm sealing.

Once the optimal graft position is achieved, the graft-clasp lock cylinder is advanced against the deployed strut ring to lock the struts into a groove on the coupler thereby ensuring that all struts are in contact with the tissue surface and apply a measurable force to the surface preventing displacement. With final positioning confirmed, a graft-clasp release cylinder separates graft-clasp subassembly from the applicator assembly. The bioabsorbable coupler and strut assembly become resorbed into the surrounding tissue, and fibroblasts enter the intradural graft to establish permanent healing of the tissue fenestration (e.g., durotomy).

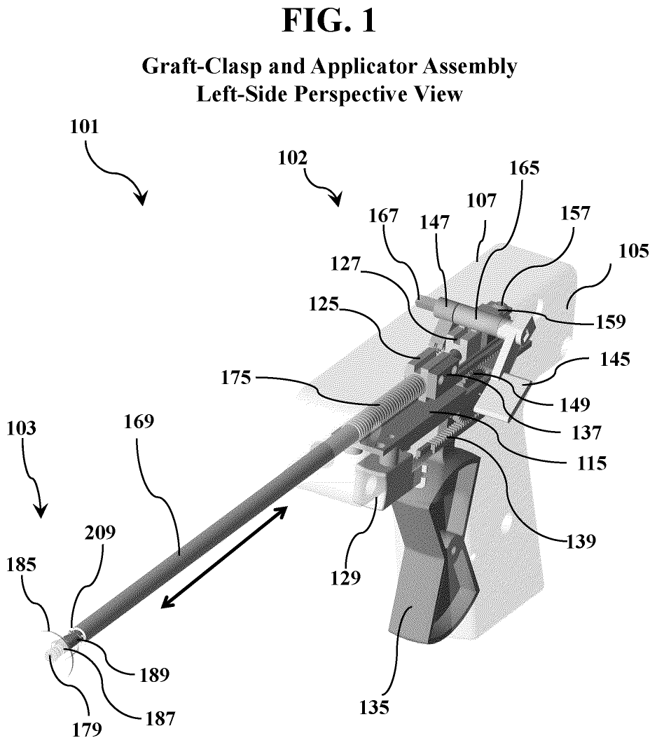

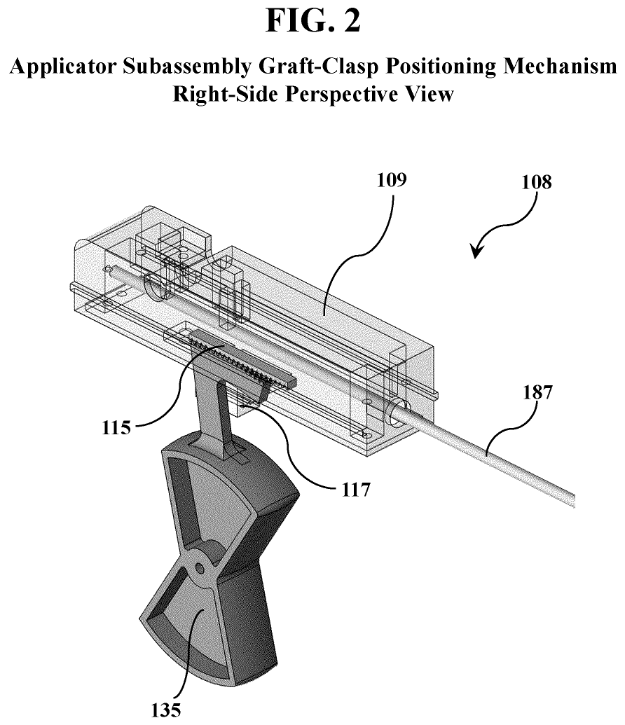

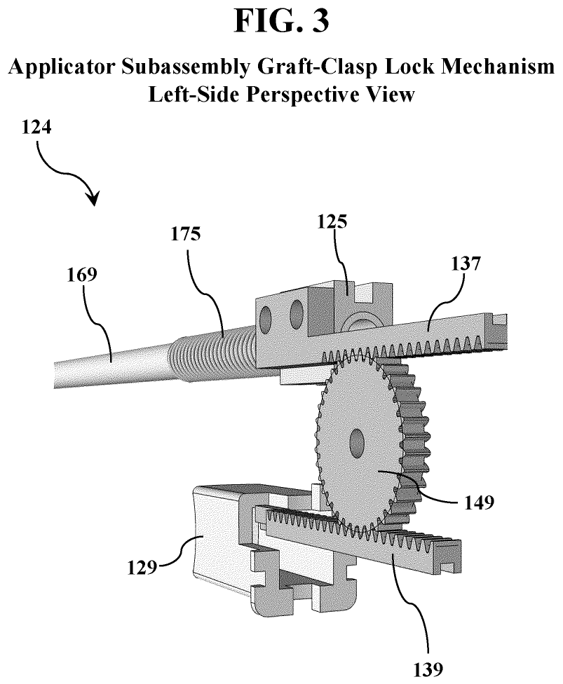

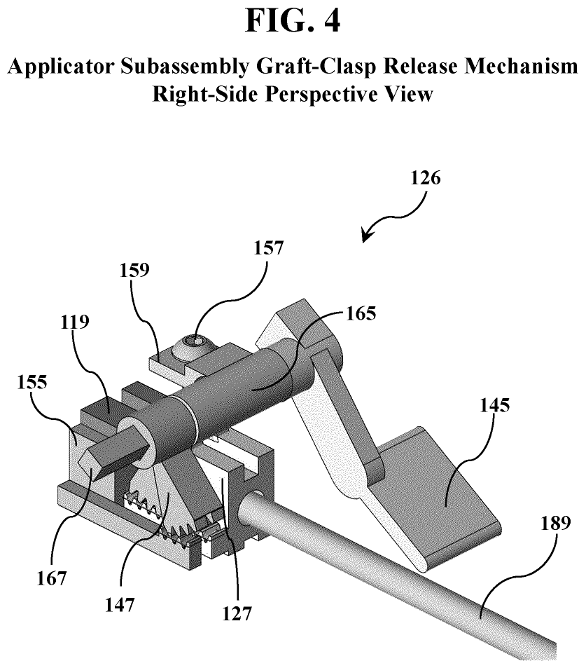

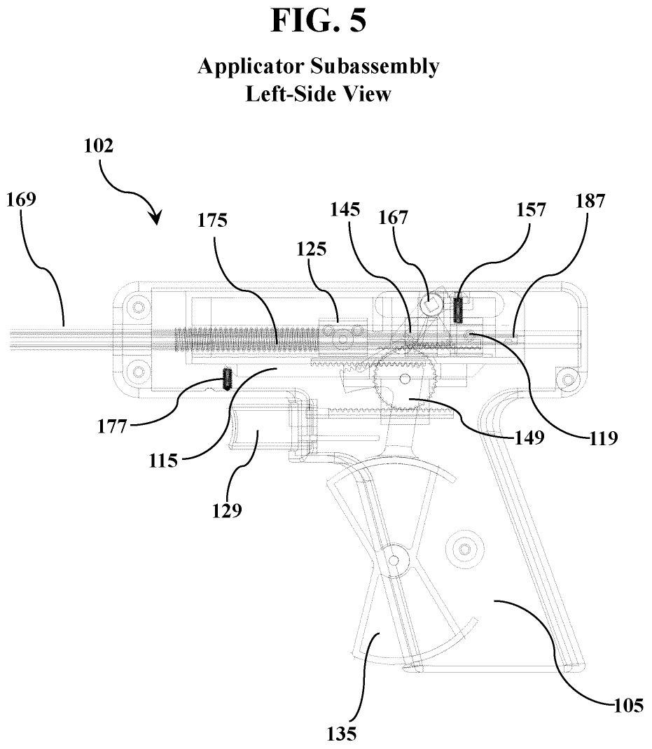

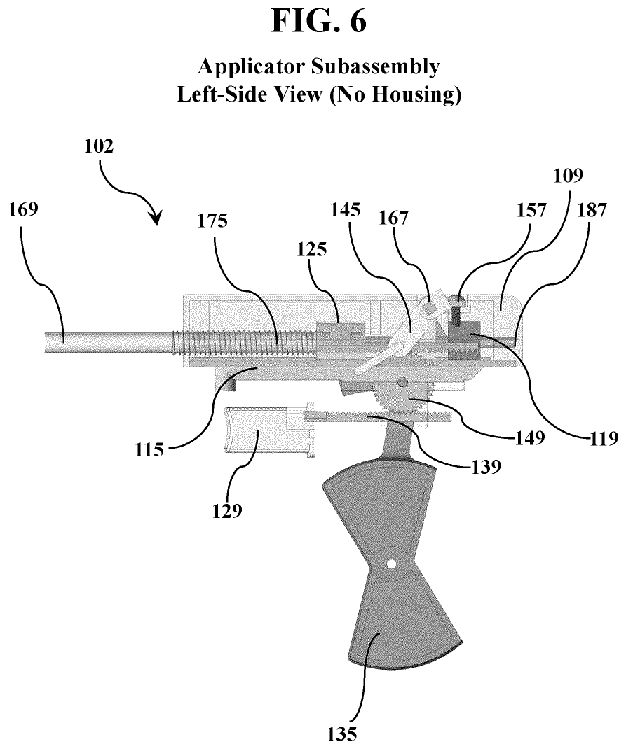

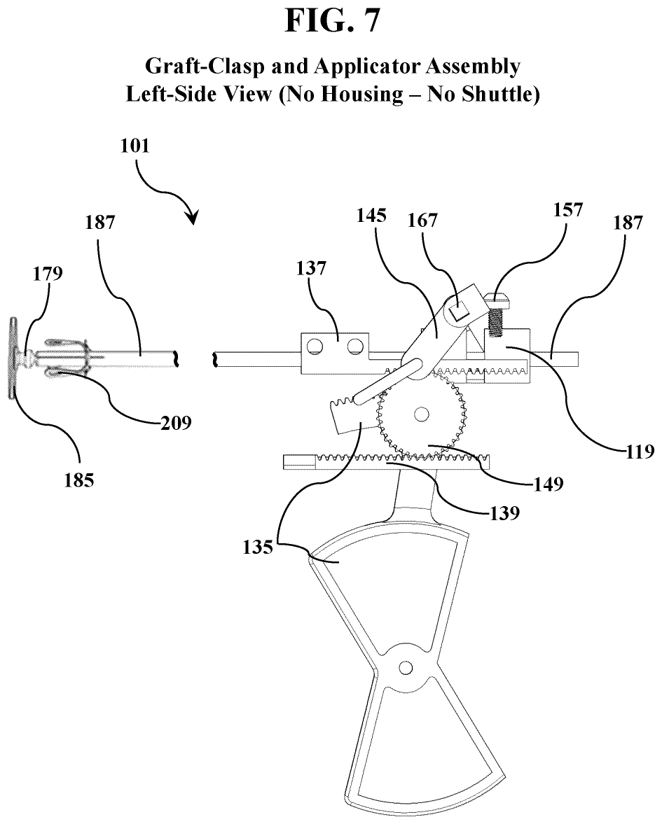

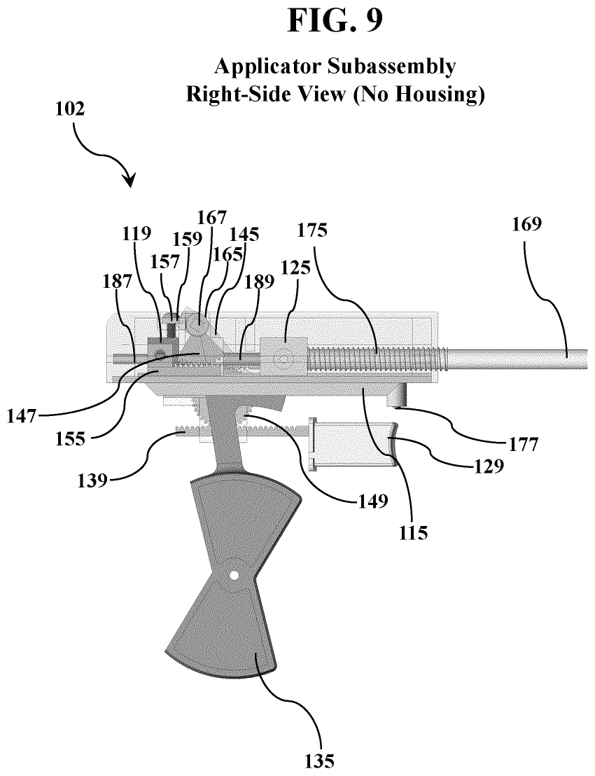

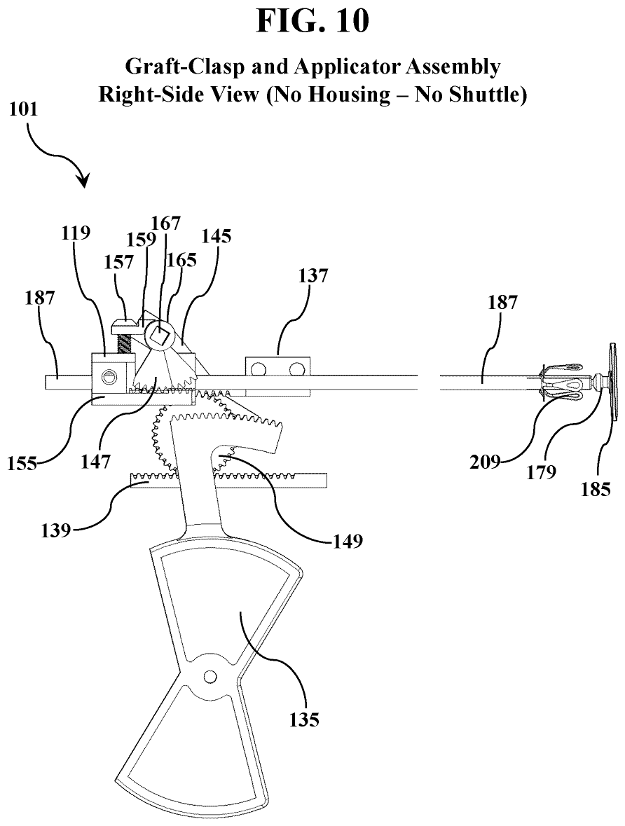

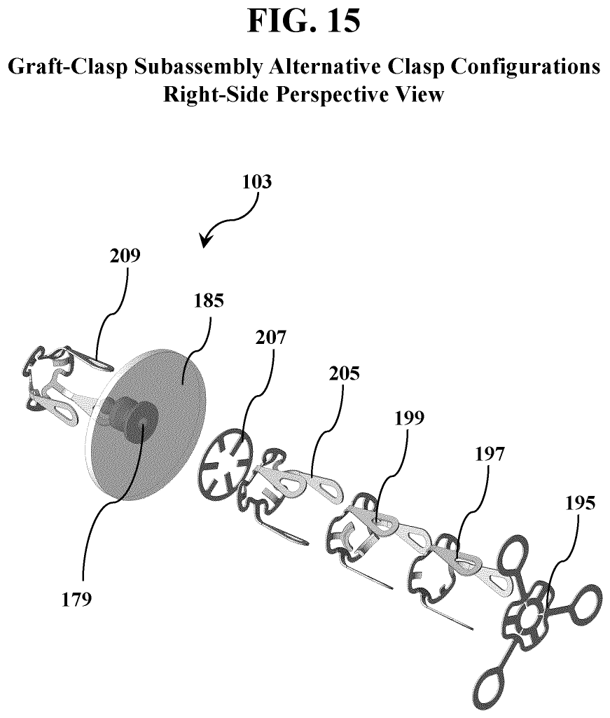

FIGS. 1-44 illustrate various aspects exemplary tissue repair and sealing devices according to various embodiments of the present disclosure. Tissue repair and sealing devices are applicator assemblies (FIGS. 1, 7, 10, and 13-14) comprising (1) an applicator subassembly (FIGS. 5-6, 8-9, and 11-12), and (2) a graft-clasp subassembly (FIGS. 15-20). Applicator subassemblies comprise (a) a graft-clasp positioning mechanism (FIG. 2), (b) a graft-clasp lock mechanism (FIG. 3), and (c) a graft-clasp release mechanism (FIG. 4). Graft-clasp subassemblies comprise (a) a foldable graft 185, (b) a retractable clasp 209, and (c) a bioabsorbable graft-clasp coupler 179 that secures the graft to the clasp. Graft-clasp positioning mechanism (FIG. 2) comprises a graft-clasp positioning cylinder 187 configured at its distal end for reversible attachment to a graft-clasp subassembly comprising a coupler that is configured for reversible attachment to the distal end of said graft-clasp positioning cylinder. Graft-clasp lock mechanism (FIG. 3) comprises a graft-clasp lock cylinder 169 and graft-clasp release mechanism (FIG. 4) comprises a graft-clasp release cylinder 189. Graft-clasp subassemblies comprise (a) a foldable graft 185, (b) a retractable clasp 209, and (c) a bioabsorbable graft-clasp coupler 179 that secures the graft to the clasp.

This tissue repair and sealing device 101 comprises:
(1) an applicator subassembly 102 and a graft-clasp subassembly 103, wherein Applicator subassembly 102 includes
   (a) a housing (left) 105 and housing (right) 107;
   (b) a graft-clasp positioning mechanism having a main shuttle top 109, a main shuttle bottom 115, a main shuttle rocker 135, a graft-clasp positioning cylinder 187, a main shuttle, gear retainer 117, and a steel metric ball-nose spring plunger 177;
   (c) a graft-clasp lock mechanism comprising a carriage frame pusher 125, a frame pusher trigger 129, a frame pusher rack top 137, a frame pusher rack bottom 139, a 20 degree Pressure Angle Plastic Gear, 149, graft-clasp lock cylinder (316 Stainless Steel) 169, and compression spring 175; and
   (d) a graft-clasp release mechanism comprising a carriage, a patch lock release 127, an a carriage eject 119, an eject lever 145, a patch lock release Gear 147, an eject rack 155, an applicator button head hex drive screw 157, a gear pivot lock 159, a gear pivot sleeve 165, a low-carbon steel bar 167, and a 316 Stainless Steel Cylinder 189; and
(2) a graft-clasp subassembly 103 comprising (a) a post and ball coupler 179, (b) a graft 185, and (c) a clasp 209.

FIGS. 1, 7, 10, and 13-14 illustrate tissue repair and sealing device assemblies 101, comprising:
(1) an applicator subassembly 102 that includes
   (a) a graft-clasp positioning mechanism having a main shuttle top 109, a main shuttle bottom 115, a main shuttle rocker 135, an graft-clasp positioning cylinder 187, a main shuttle, gear retainer 117, and a steel metric ball-nose spring plunger 177;
   (b) a graft-clasp lock mechanism comprising a carriage frame pusher 125, a frame pusher trigger 129, a frame pusher rack top 137, a frame pusher rack bottom 139, a 20 degree Pressure Angle Plastic Gear, 149, graft-clasp lock cylinder 169, and compression spring 175; and
   (c) a graft-clasp release mechanism comprising a carriage, a patch lock release 127, an a carriage eject 119, an eject lever 145, a patch lock release Gear 147, an eject rack 155, an applicator button head hex drive screw 157, a gear pivot lock 159, a gear pivot sleeve 165, a low-carbon steel bar 167, and a graft-clasp release cylinder 189; and
(2) a detachable graft-clasp subassembly 103 that includes
   (a) a coupler (post and ball) 179,
   (b) a graft 185, and
   (c) a clasp 209.

FIG. 2 is a right-side perspective view illustrating an applicator subassembly graft-clasp positioning mechanism having a main shuttle top 109, a main shuttle bottom 115, a main shuttle rocker 135, a graft-clasp positioning cylinder (graft-clasp positioning cylinder) 187, a graft-clasp positioning main shuttle top 109, a graft-clasp positioning main shuttle, bottom 115, a graft-clasp positioning main shuttle, rocker 135, a graft-clasp positioning main shuttle, gear retainer 117, and a steel metric ball-nose spring plunger 177.

FIG. 3 is a left-side perspective view illustrating an applicator subassembly graft-clasp lock mechanism comprising a graft-clasp lock cylinder (316 Stainless Steel) 169, graft-clasp lock carriage, frame pusher 125, graft-clasp lock frame pusher, trigger 129, graft-clasp lock frame pusher rack 137, graft-clasp lock frame pusher rack, bottom 139, graft-clasp lock 20 degree pressure angle plastic gear 149, graft-clasp lock cylinder (316 Stainless Steel) 169, and graft-clasp lock compression spring 175.

FIG. 4 is a right-side perspective view illustrating an applicator subassembly graft-clasp release mechanism comprising: graft-clasp release cylinder 189, graft-clasp release carriage patch lock release 127, graft-clasp release carriage eject 119, graft-clasp release eject lever 145, graft-clasp release patch lock release gear 147, graft-clasp release eject rack 155, graft-clasp release button head hex drive screw 157, graft-clasp release gear pivot lock 159, graft-clasp release gear pivot sleeve 165, graft-clasp release low-carbon steel bar 167.

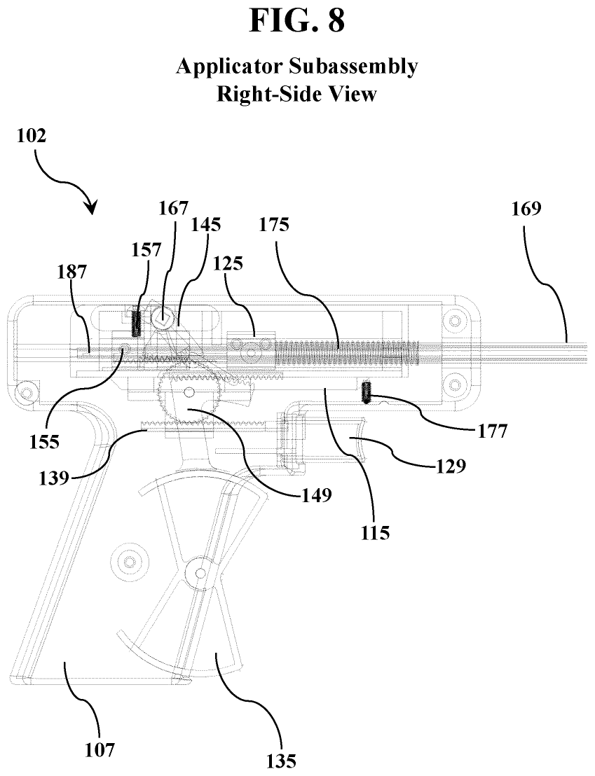

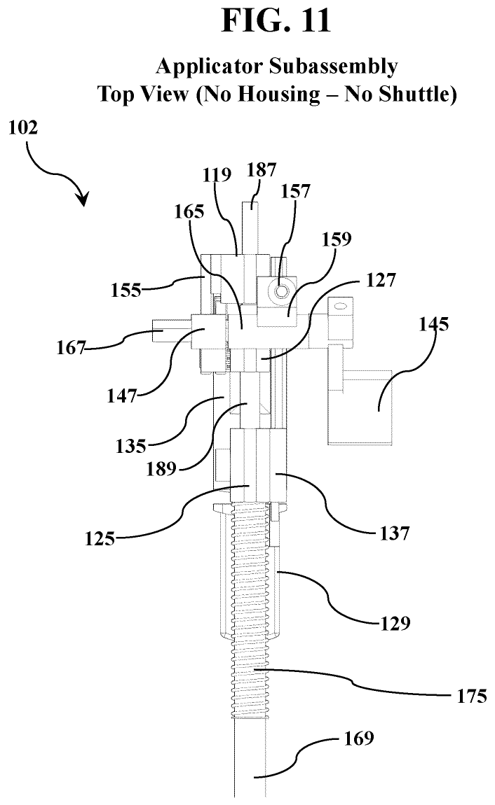

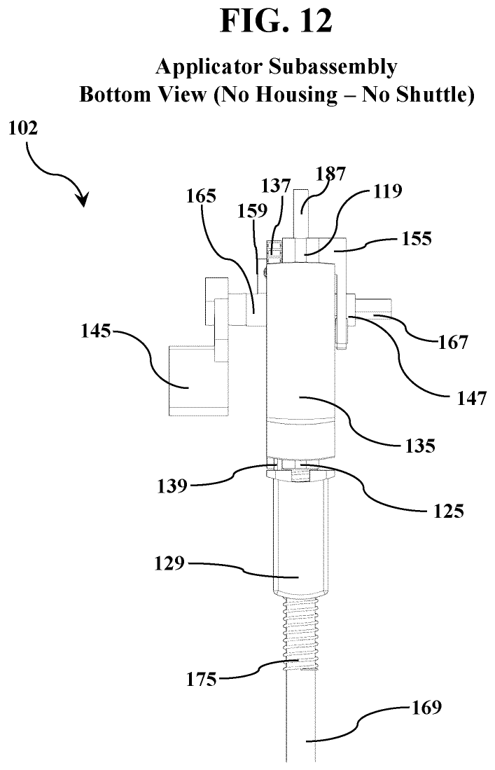

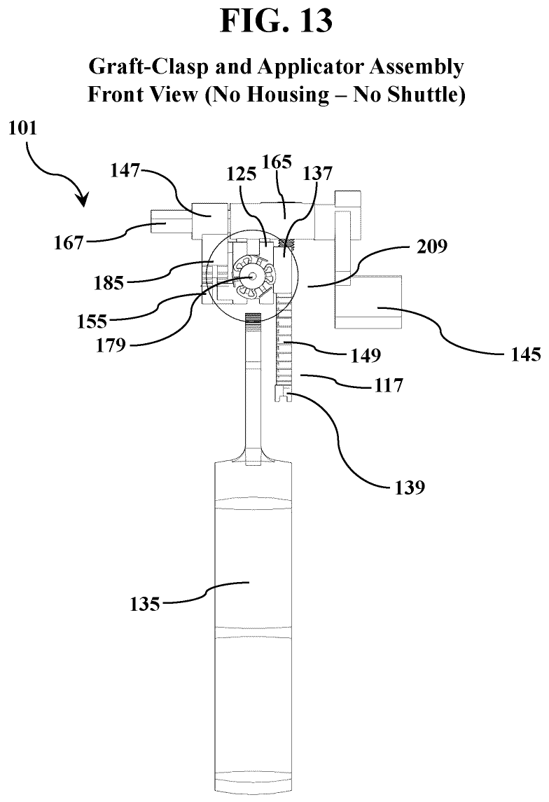

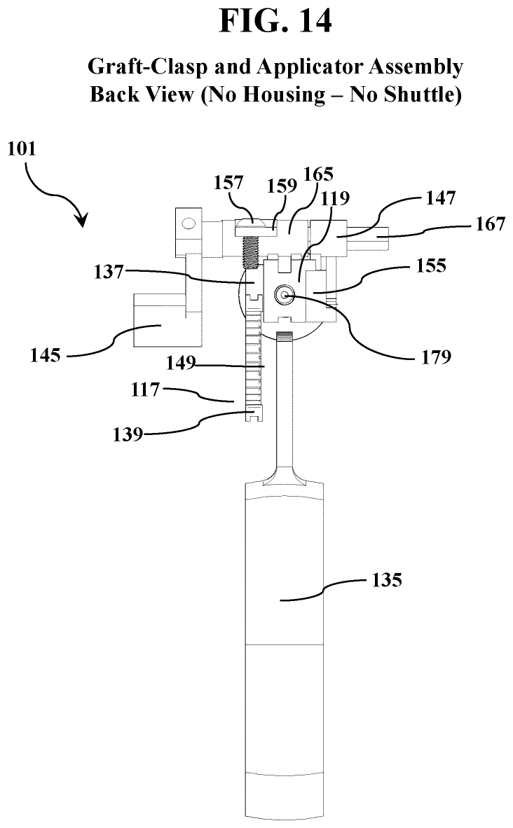

FIGS. 5-7 are left-side views, FIGS. 8-10 are right-side views, FIG. 11 is a top view, FIG. 12 is a bottom view, FIG. 13 is a front view, and FIG. 14 is a back view illustrating (1) an applicator subassembly 102 comprising (a) a graft-clasp positioning mechanism having a main shuttle top 109, a main shuttle bottom 115, a main shuttle rocker 135, a graft-clasp positioning cylinder 187, a main shuttle, gear retainer 117, and a steel metric ball-nose spring plunger 177; (b) a graft-clasp lock mechanism comprising a carriage frame pusher 125, a frame pusher trigger 129, a frame pusher rack top 137, a frame pusher rack bottom 139, a 20 degree Pressure Angle Plastic Gear, 149, graft-clasp lock cylinder (316 Stainless Steel) 169, and compression spring 175; and (c) a graft-clasp release mechanism comprising a carriage, a patch lock release 127, an a carriage eject 119, an eject lever 145, a patch lock release Gear 147, an eject rack 155, an applicator button head hex drive screw 157, a gear pivot lock 159, a gear pivot sleeve 165, a low-carbon steel bar 167, and a 316 Stainless Steel Cylinder 189.

In these tissue repair and seal devices, a graft-clasp lock cylinder (169) holds the graft-clasp subassembly and surrounds the Graft-Clasp Positioning cylinder (187) and the graft-clasp release cylinder (189). The graft and clasp are sequentially deployed out of the graft-clasp lock cylinder as the Graft-Clasp Positioning cylinder (187) slides forward (the graft can also be pulled back into the cylinder from under the dura after deployment by backward movement of the graft-clasp positioning cylinder). After the graft is deployed a push rod for external clasp pushes the external clasp out of the clasp lock cylinder, then advances the external clasp ring along the graft-clasp positioning cylinder and over the central peg of the graft onto the locking groove of the peg.

The graft is attached to graft-clasp positioning cylinder by a central peg which couples to a ball and socket coupling joint (which enables rotation of the graft). The graft-clasp positioning cylinder moves forward and back within the graft-clasp lock cylinder to extrude and deploy the graft out of the cylinder, and to retract it back into the cylinder if necessary. The graft-clasp positioning cylinder-to-graft coupling allows stabilization of the graft in place below the dura as the push rod advances the external clasp ring along the graft-clasp positioning cylinder until the external clasp central ring locks onto the locking groove of the graft member central peg. At the satisfactory conclusion of the graft deployment and external graft-clasp locking, the central-most separation rod slides forward to disarticulate the graft-clasp positioning cylinder—graft coupling, and the applicator is removed from the field.

FIG. 15 is a right-side perspective view of exemplary graft-clasp subassemblies 103 according to embodiments of the tissue repair and sealing devices disclosed herein (shown with alternative clasp configurations).

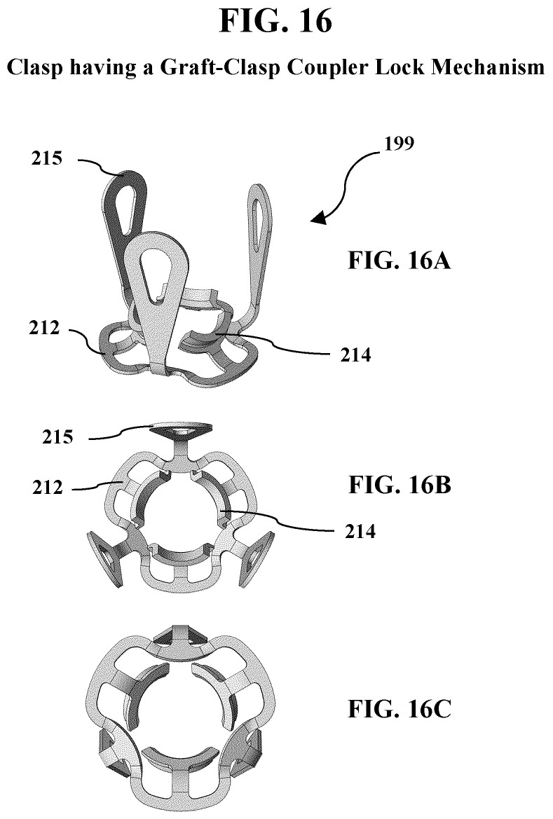

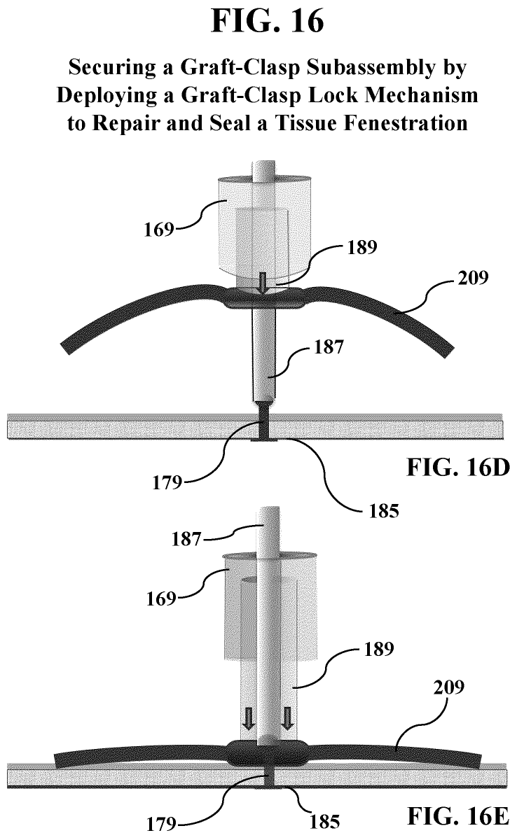

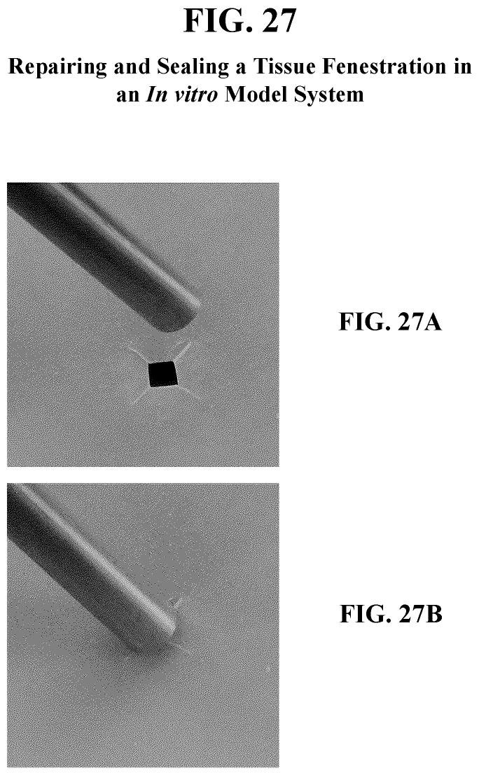

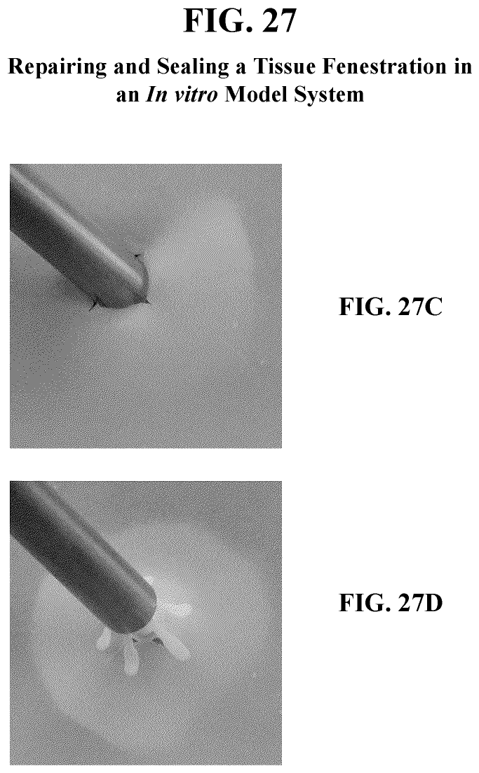

FIG. 16 are perspective views of the external clasp (FIG. 16A-C) and right side schematic views (FIG. 16C-D) of the graft-clasp subassembly locking mechanism according to embodiments of the tissue repair and sealing devices disclosed herein. FIGS. 16A-C are side, top and bottom views of the external clasp 209 showing a multiplicity of radial arms with wide oval distal segments 215 emanating from a central ring 212. Within the central ring are numerous short radial struts 214 which bend to stabilize the external clasp as it slides proximally along the applicator subassembly toward the graft-clasp subassembly, then revert to their original configuration to lock the ring to the base of the coupler. FIG. 27 C-D are schematic side views of the external clasp locking mechanism as the external clasp 209 is advanced proximally along the applicator subassembly by a push rod 189, and locks into a groove in a post, ball, and flange coupler 179 according to embodiments of the tissue repair and sealing devices disclosed herein.

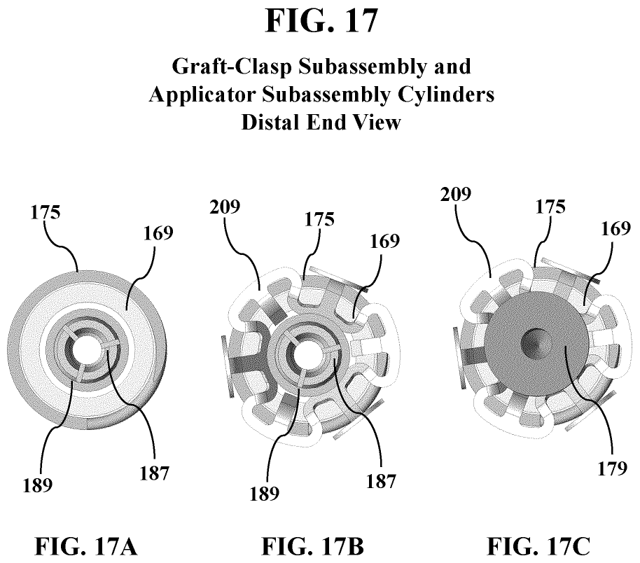

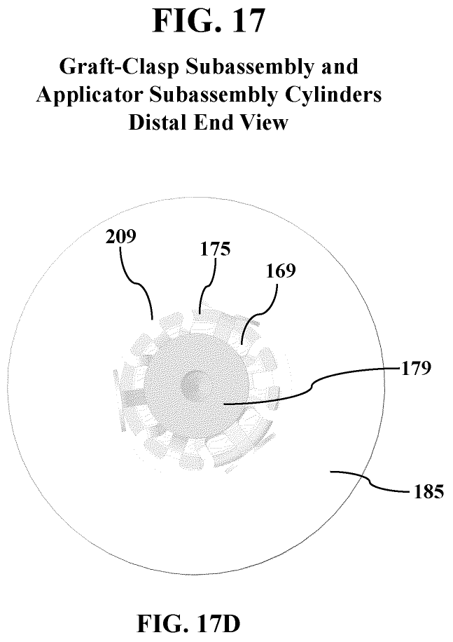

FIG. 17A-D are distal end views of an exemplary graft-clasp subassembly releasably attached to the distal end of an applicator subassembly graft-clasp positioning cylinder according to embodiments of the tissue repair and sealing devices disclosed herein.

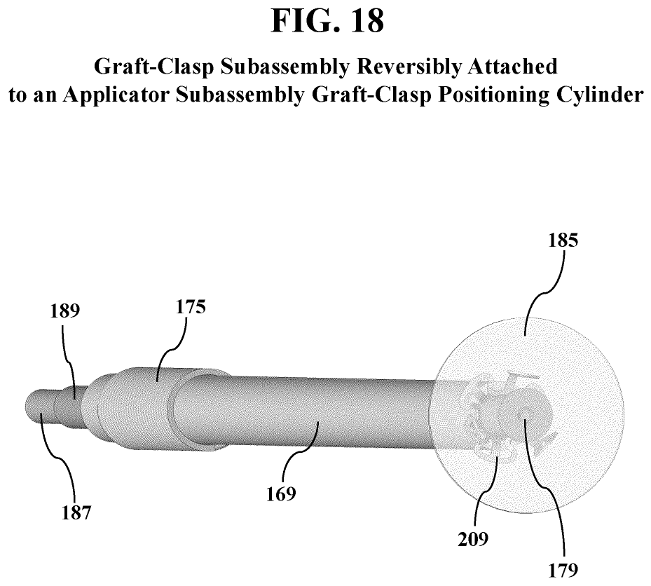

FIG. 18 is a right-side view showing the attachment of an exemplary graft-clasp subassembly 185 via coupler 179 to a ball socket cylinder 187 of an applicator subassembly according to embodiments of the tissue repair and sealing devices disclosed here.

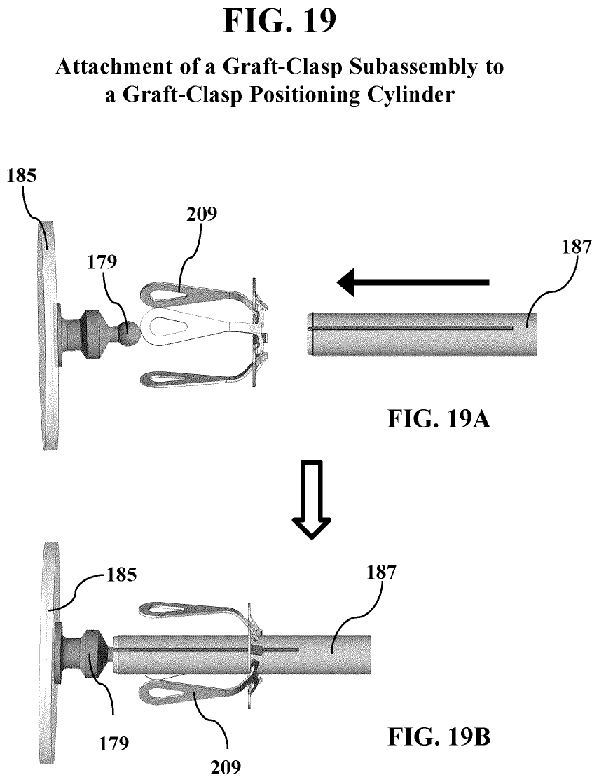

FIGS. 19A-19B are left-side views that show the external clasp 209 mounted on the applicator subassembly 187 which is releasably attached to the coupler 179 of the graft-clasp subassembly prior to folding the graft and clasp distally, away from the applicator handle, and loading into a graft-clasp lock cylinder 169.

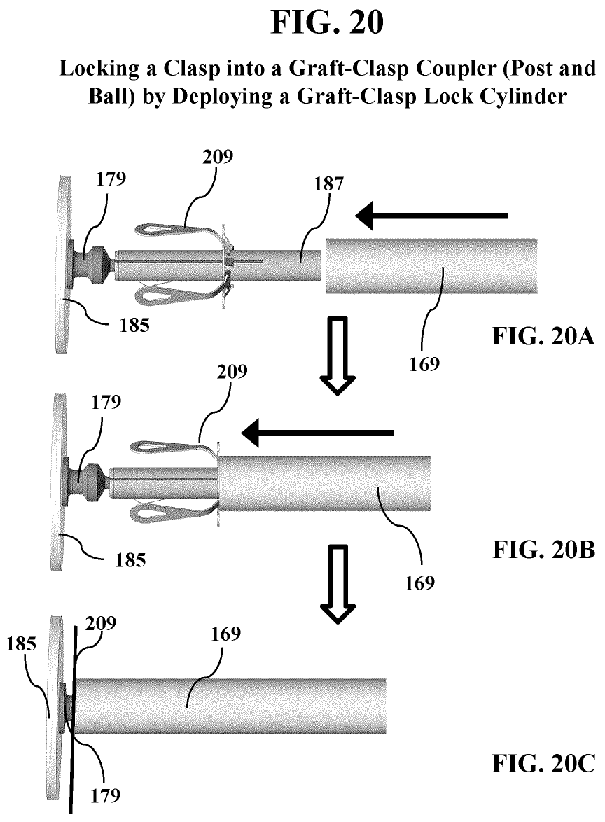

FIGS. 20A-20C are left side views of a graft-clasp positioning cylinder 187 attached to the coupler 179 of a graft-clasp subassembly 103 and an external clasp 209 mounted on the distal end of graft-clasp positioning cylinder with radial struts folded toward the graft and distally from the applicator handle (FIG. 20A) and enclosed within graft-clasp lock cylinder 169.

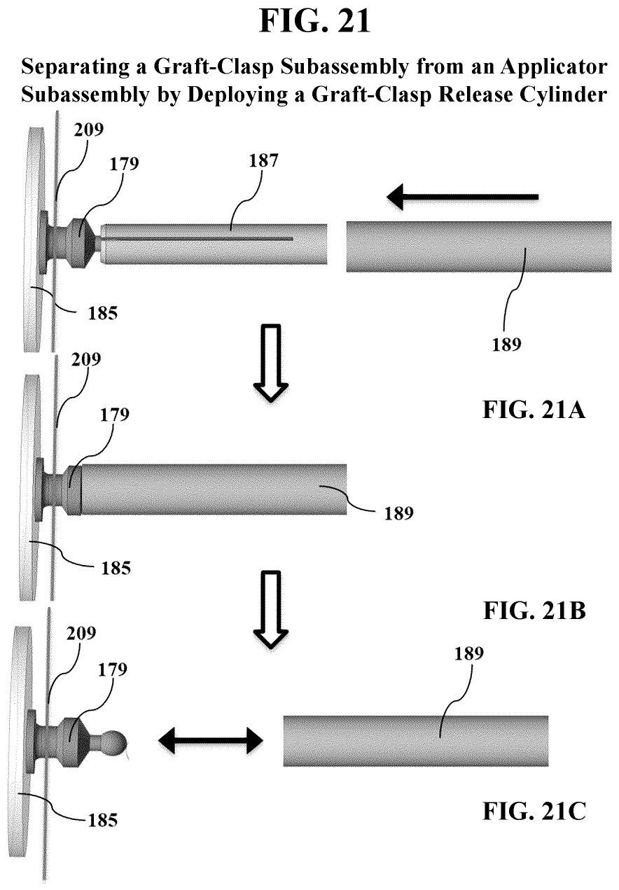

FIG. 21 is a left side view showing the attachment (FIG. 21A) and release (FIG. 21C) of a graft-clasp coupler 179 from the distal end of graft-clasp positioning cylinder 187 to the coupler of graft-clasp subassembly 103 by proximal and distal advancement of a deployable graft-clasp lock cylinder 189 according to embodiments of the tissue repair and sealing devices disclosed herein.

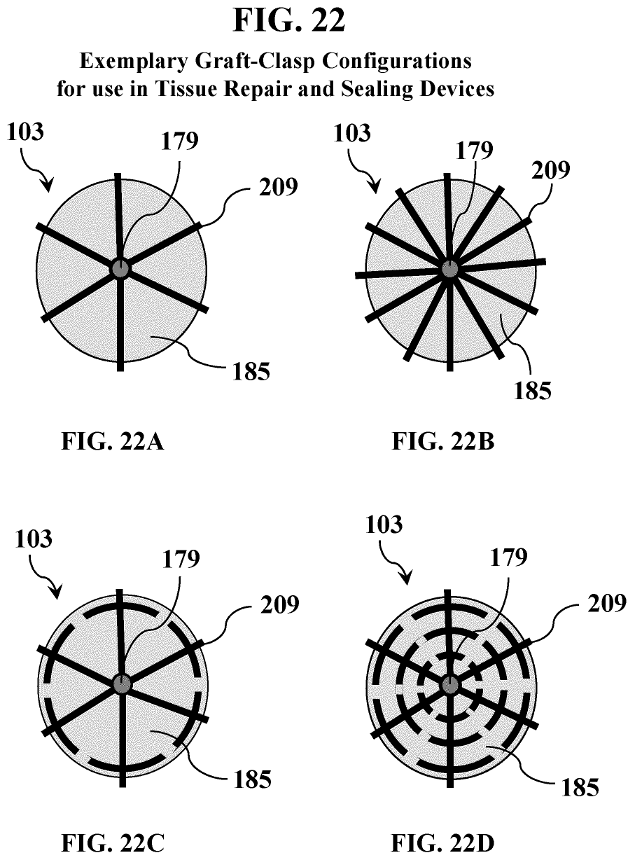

FIGS. 22A-22D shows a variety of potential external clasp strut configurations for a graft-clasp subassembly 103 to, for example, increase the surface area between the clasp struts and tissue outer surface, thereby providing more force to position and secure the graft.

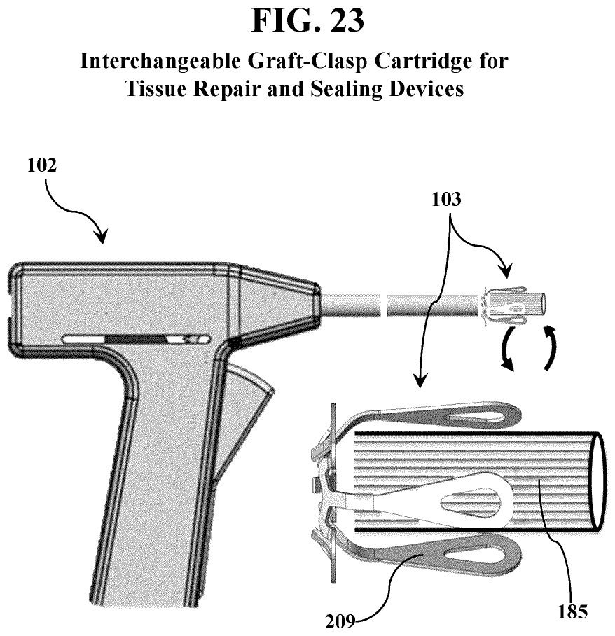

FIG. 23 is a tissue repair and sealing device applicator subassembly 102 shown with an interchangeable graft-clasp subassembly self-contained cassette 103 comprising a graft that can be quickly substituted on the applicator subassembly handpiece during a surgical procedure.

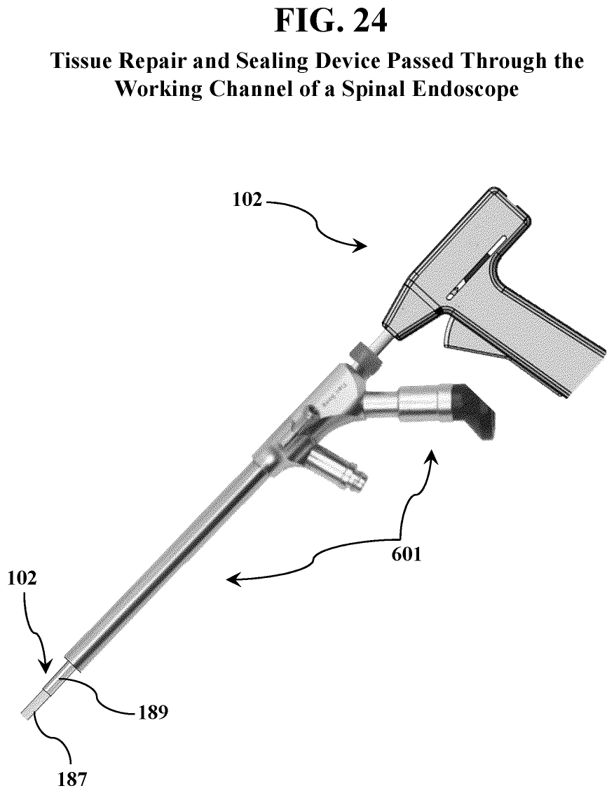

FIG. 24 shows a tissue repair and sealing device applicator subassembly 102 for use in combination with a spinal endoscope 601 for accessing the spine via a working channel.

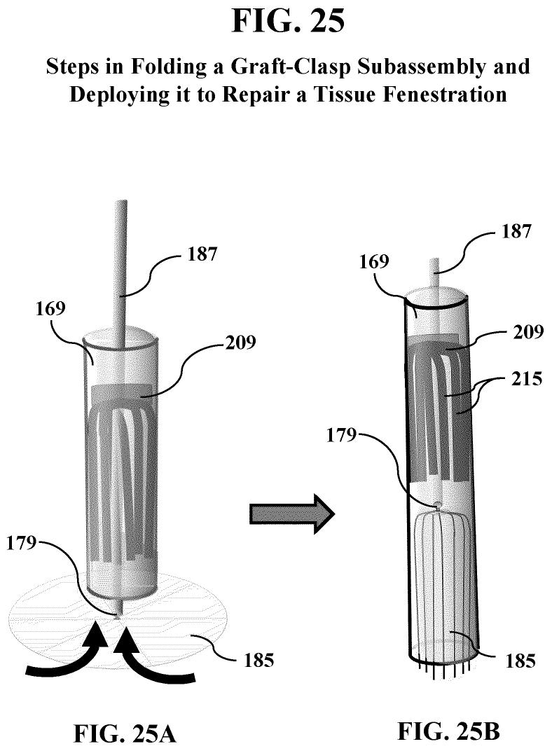

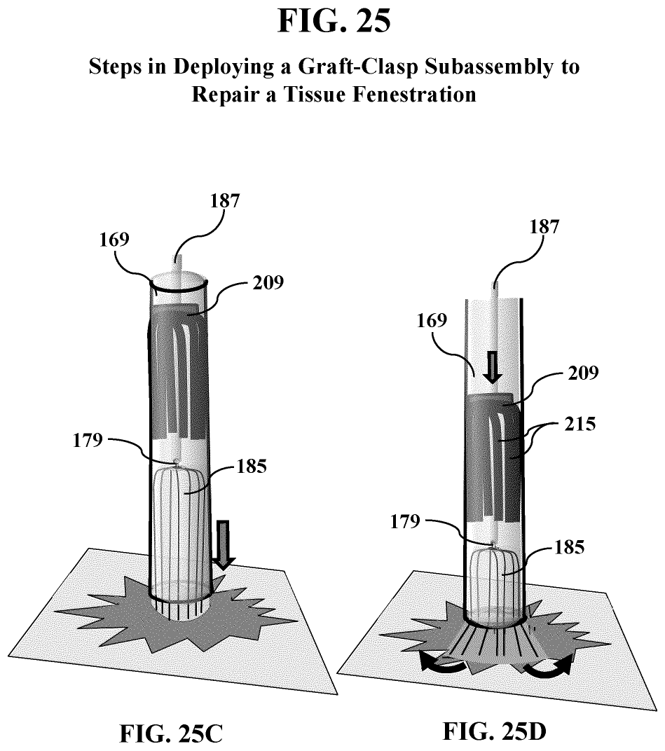

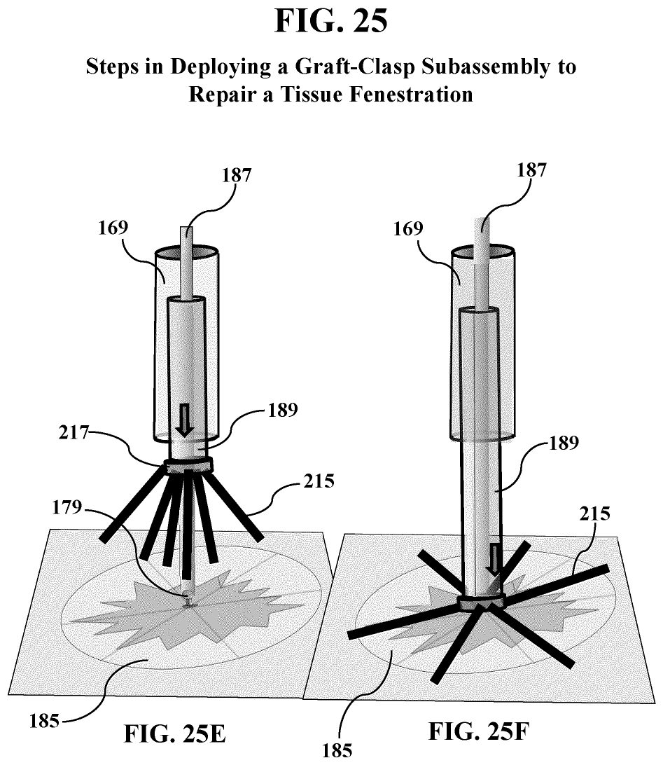

FIG. 25 is a schematic representation of the steps involved in deploying a graft-clasp subassembly 102 to repair and seal a tissue fenestration according to embodiments of the tissue repair and sealing devices disclosed herein and showing that both the graft 185 and the external clasp struts 215 are both folded in the same direction and away from the distal end of the applicator subassembly 102. In FIG. 25A, the external clasp has been loaded into the outer graft-clasp lock cylinder with the ring proximally and the radial struts folded and pointing distally. Graft 185 is attached to the central graft-clasp positioning cylinder 187 via a coupler 179 (e.g., ball-socket) and the central graft-clasp positioning cylinder is withdrawn into the graft-clasp lock cylinder 169, where it resides in a folded configuration (FIG. 25B).

In FIG. 25C the central graft-clasp positioning cylinder is attached to the folded graft and used to advance the graft distally until 1-2 mm of graft is extruded from the graft-clasp lock cylinder, and the tip of the folded graft is passed into the tissue fenestration. In FIG. 25D the graft is advanced distally into the sub-membrane space and spontaneously unfolds into its original flat configuration. The graft-clasp positioning cylinder 187 remains at the orifice of the membrane defect which causes the graft expansion to occur in close proximity (2-3 mm) to the inner surface of the tissue.

In FIG. 25E the expanded graft is pulled a against the inner membrane surface and inspected and moved, if necessary, to completely occlude the fenestration. If the graft is the wrong size or shape, it can be withdrawn back into the graft-clasp lock cylinder and exchanged for a different graft. In FIG. 25E, the graft-clasp positioning cylinder is withdrawn as the graft-clasp lock cylinder is deployed to extrude the external clasp ring thereby allowing the external clasp struts 215 to partially expand. In FIG. 25F, with the graft positioned on the inner surface under continuous view by the surgeon, the external clasp ring 217 is advanced as the struts slide across the membrane surface, and the clasp ring is locked onto coupler 179 (e.g., via a locking groove in the coupler that receives a locking member in the clasp) and thereby maintaining the force that the struts apply to the membrane and stabilizing the position of the graft.

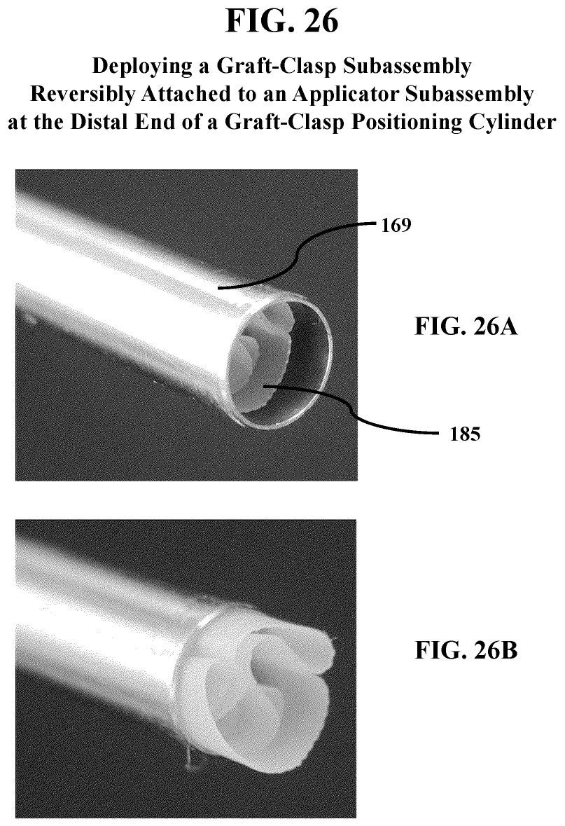

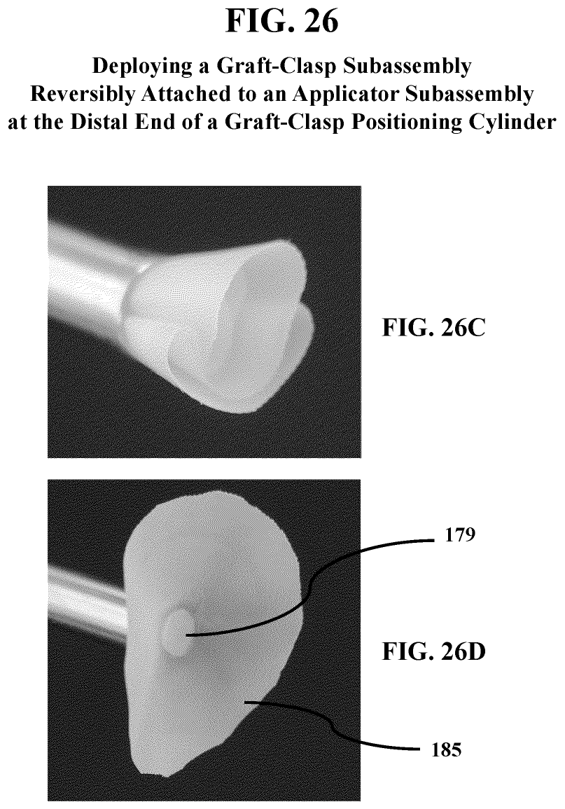

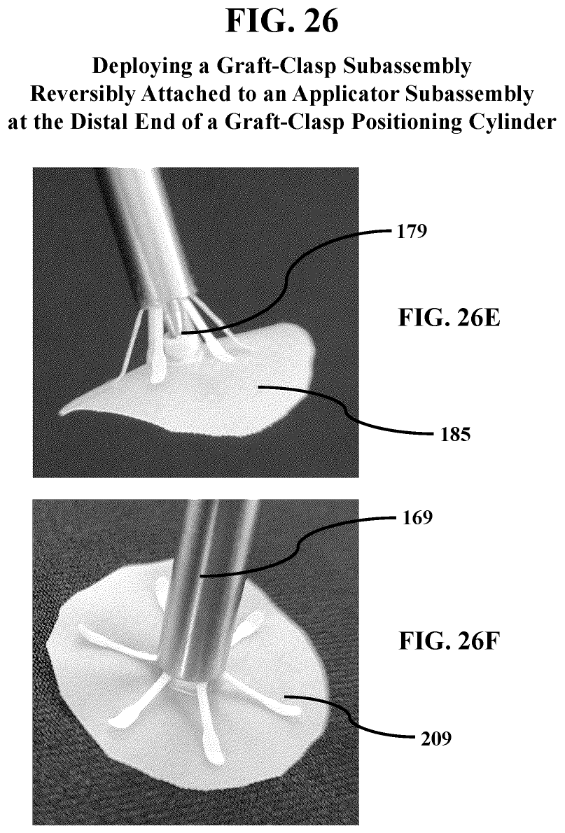

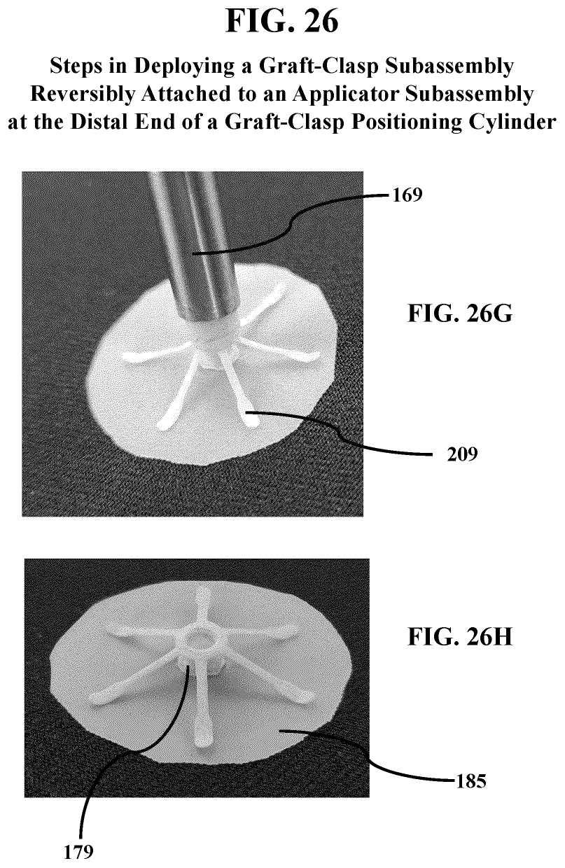

FIGS. 26A-26H are photographic illustrations of the deployment of graft-clasp subassembly with a tissue repair and sealing device without the presence of a tissue membrane. In FIG. 26A, the graft can be seen folded in the distal end of the graft-clasp lock cylinder 169. The graft is progressively advanced out of the graft-clasp lock cylinder by deploying a graft-clasp positioning cylinder (FIGS. 26B-26C) and spontaneously expands to its original flat configuration (FIG. 26D). In FIG. 26E, the struts of the external clasp are advanced out of the distal end of the graft-clasp lock cylinder 169 and the clasp ring 217 locks into a groove in the coupler 179, thereby maintaining force of the struts against the outer tissue surface of to stabilize the position of the graft and create a pressure-resistant, watertight seal. The curved tips of the clasp struts facilitate smooth expansion of the struts as they slide across the membrane surface during the locking procedure.

FIG. 27 is a photographic illustration the deployment of a tissue repair and sealing device graft-clasp subassembly to seal and repair an in vitro model silastic membrane fenestration. In FIG. 27A-26B, the distal end of the graft-clasp lock cylinder is placed at the 10 mm×10 mm fenestration. In FIG. 27C the graft-clasp lock cylinder is passed slightly through the fenestration and the graft is released by deploying the graft-clasp positioning cylinder to expand on the inner surface of the membrane. After full deployment (FIG. 26D), the graft is pulled back up against in inner surface of the membrane and the biodegradable external clasp struts are advanced out of the graft-clasp lock cylinder and the clasp ring forced into a groove in the coupler, thereby maintaining force of the struts against the outer surface of the membrane to stabilize the position of the graft.

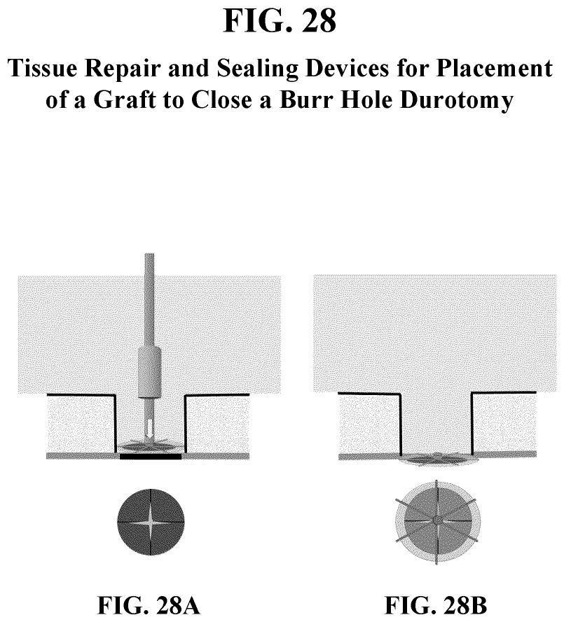

FIG. 28 illustrates embodiments of the tissue repair and sealing devices disclosed herein for use in the repair and sealing a dural incision at the base of a burr hole in the skull. Burr holes are 10-15 mm diameter holes drilled to a depth of 1-2 cm) for placement of catheters or electrodes, drainage of fluid collections or tissue biopsies. The dura at the base of the hole is incised and can be difficult to repair due to the limited diameter of the hole. FIG. 28A shows the tissue repair and sealing device above the dura at the base of a burr hole (upper) and the cruciate incision in the dura (lower). In FIG. 28B (upper and lower) the graft is placed beneath the dura and secured with an extradural clasp.

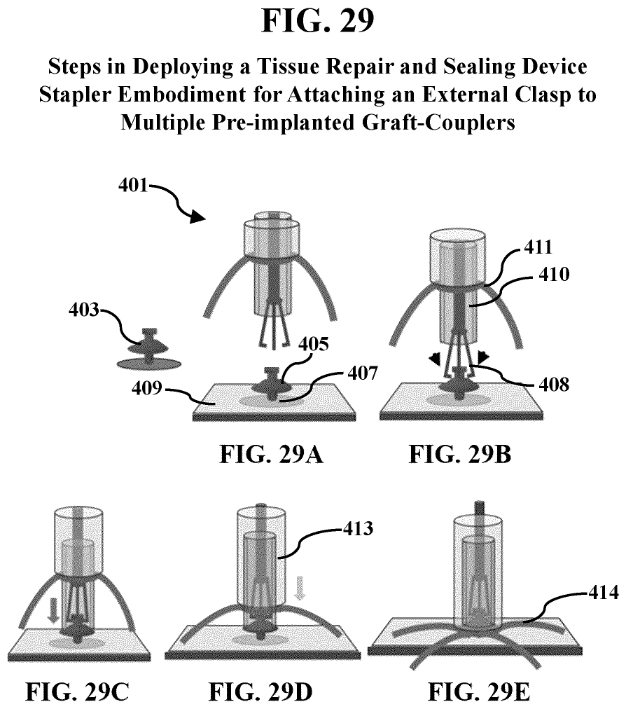

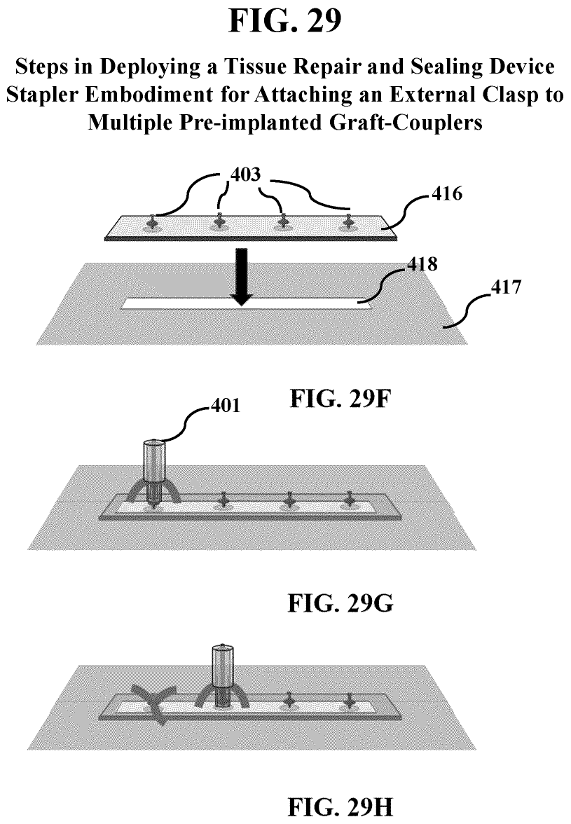

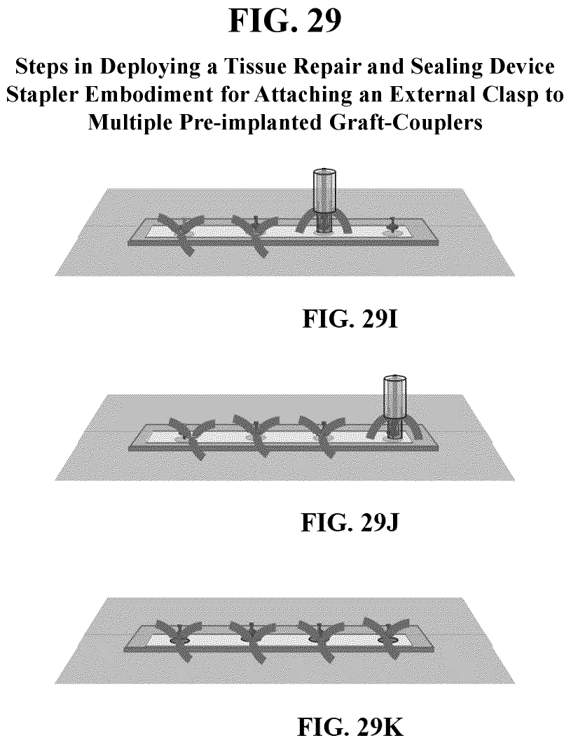

FIGS. 29A-K show a modification of the device to function separately from the graft as an external clasp attachment assembly 401 in a stapler-like fashion by sequentially attaching several external clasps to multiple pre-implanted couplers in dural grafts for the rapid closure of long open cranial or spinal incisions. FIGS. 29A-29E show the method by which the attachment assembly attaches an external clasp to individual pre-implanted couplers 403 by grasping the coupler peg (FIG. 29A-B) and advancing the external clasp over the coupler peg FIG. 29C-E) to lock the clasp into place. FIGS. 29F-29K shows the method by which a rectangular graft of dural substitute material is secured along a dural incision with multiple clasps. The stapler clasp attachment is loaded with multiple external clasps along its central applicator tube (not shown) that drop into the attachment position sequentially as the prior clasp is attached. In FIG. 29A the coupler is inserted into a dural substitute graft 409 with multiple pre-implanted couplers with the base plate beneath the graft and with a ball peg 405 and locking groove 407 above the graft. The external clasp attachment assembly consists of a central shaft with grasping hooks 408 at the distal end, contained within a sliding grasp hook closure cylinder 410, on the surface of which the ring of the external clasp 411 slides distally and proximally to close and open the grasping hooks, respectively. The external clasp attachment assembly is positioned just above the coupler (FIG. 29B) and the grasp hook closure cylinder moved proximally to close the grasping hooks onto the grasping peg (FIG. 29C), after which the external clasp push rod 413 advances the ring of the external clasp distally (FIG. 29C) until it locks in the groove at the base of the coupler peg, thereby securing the graft material to the adjacent dura by the force of the external clasp struts 414. Sliding the grasp hook closure cylinder proximally releases the grasping hooks from the coupler peg and another external clasp drops into the breach of the external clasp attachment assembly, which then moves to the next coupler to attach another external clasp as below. FIGS. 29F-29K show the method by which several external clasps are sequentially attached to the couplers in the graft to secure it beneath a long incision in the dura 417. In FIG. 29F a long rectangular graft 416 with multiple couplers 403 pre-implanted is placed through a dural incision 418 with the graft and coupler bases below the dura and the coupler pegs protruding through the incision to be visible above. In FIGS. 29G-29K the external clasp attachment assembly 401 is used to attach and secure an external clasp to each peg sequentially, such that the graft below the dural incision is secured fixed and watertight (FIG. 29K).

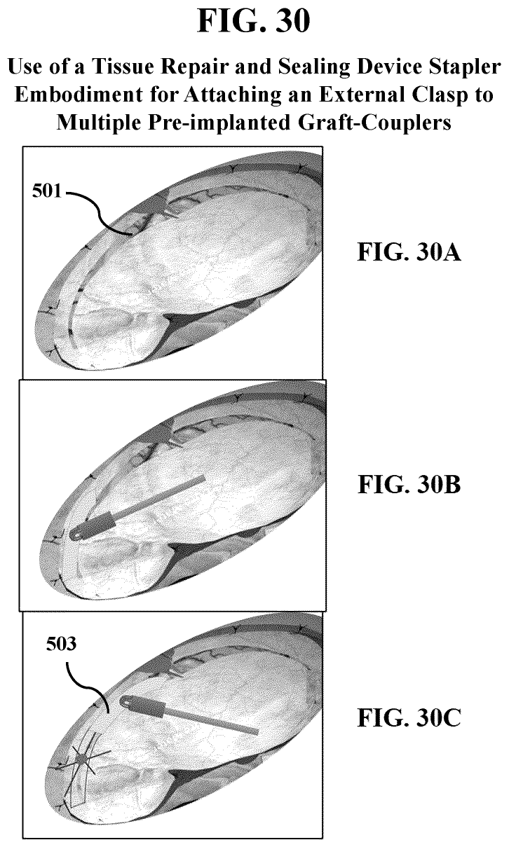

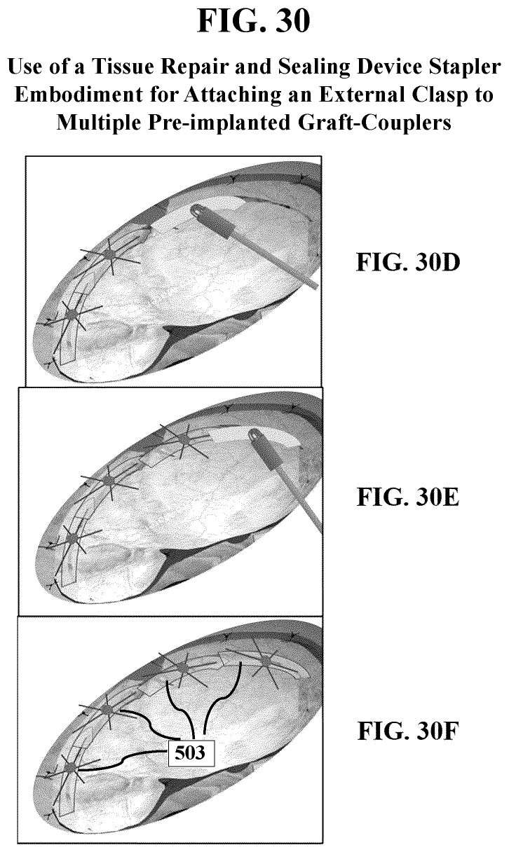

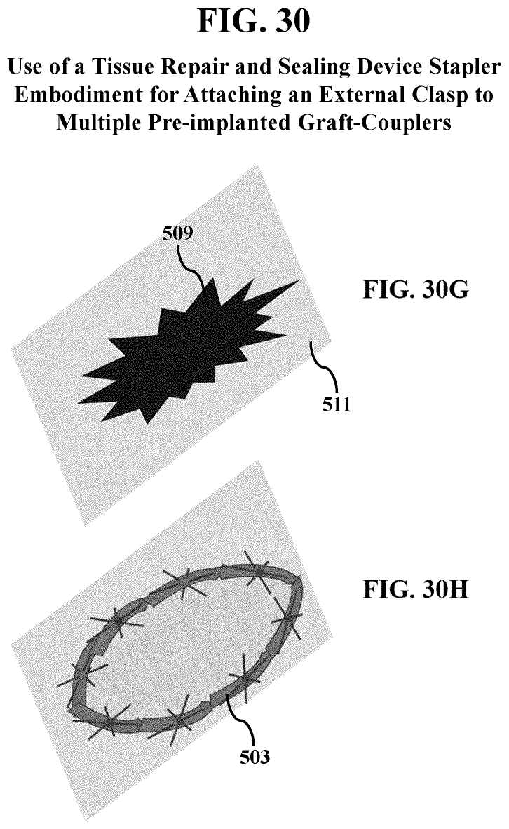

FIG. 30A-30F shows repair and sealing of a long open surgery craniotomy dural incision 501 by application of multiple individual curved rectangular grafts 503 placed sequentially end-to-end under the dura. In a similar fashion, a duroplasty to repair a large defect 509 in the dura 511 is performed (FIG. 30G) by securing a piece of graft material sized to the defect with multiple circumferential individual curved rectangular 503 (FIG. 30H).

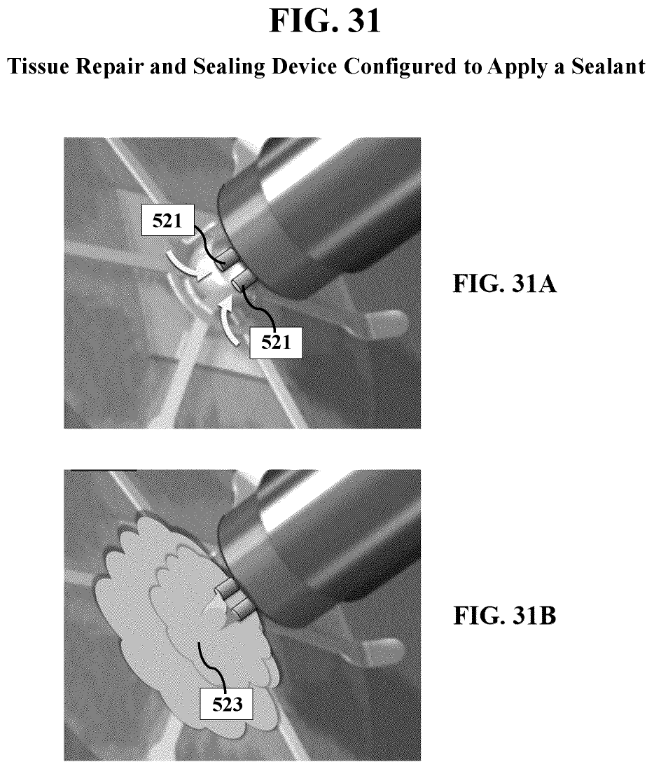

FIG. 31A shows a modification of a tissue repair and sealing device applicator for delivering tissue sealants such as fibrin (Tisseal®) or polymer gels (DuraSeal®) as sealing adjuncts to the repair and seal site following deployment of the graft. Tissue sealants of various compositions may be administered as 2-component solutions through two tubes 521 which are embedded in the wall of the device applicator tube. In FIG. 31B the components mix at the application site 523 under direct vision without changing the field of view and quickly polymerize in situ to form an adherent polymer layer over the graft at the repair site.

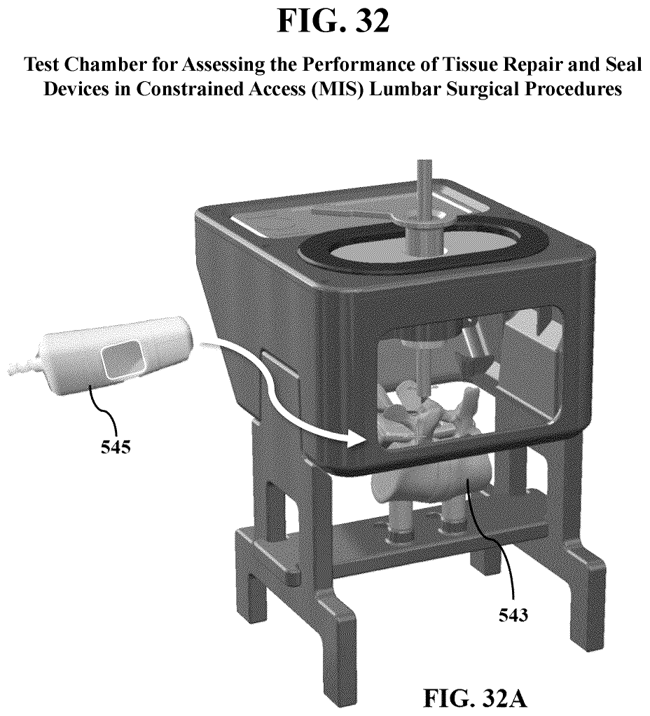

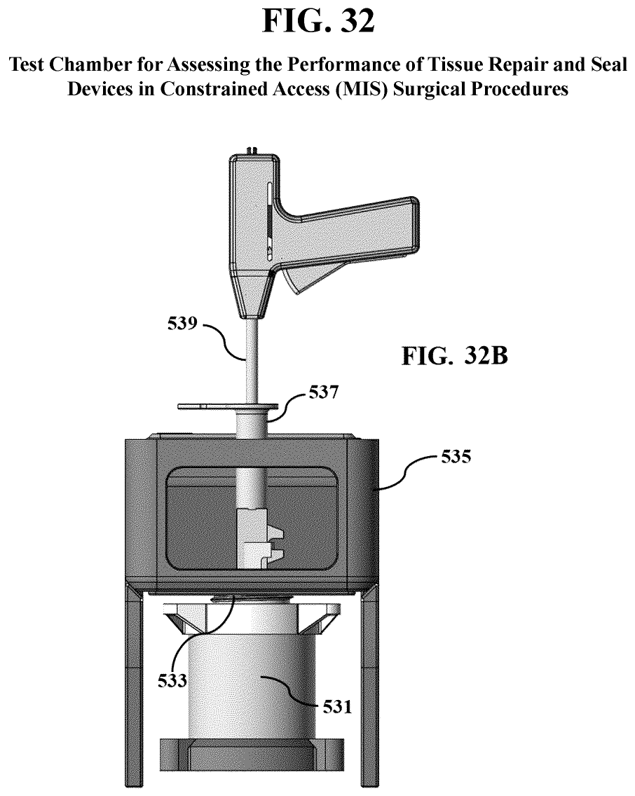

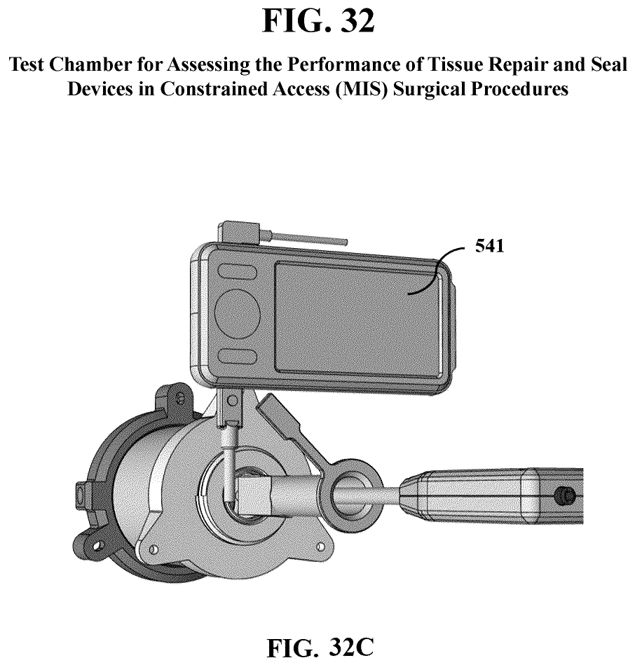

FIG. 32A (lateral view) and FIG. 32B (top view) show a test pressure chamber and simulator frame for assessing the performance of tissue repair and seal devices in MIS procedures characterized by limitations in surgical access to the target tissue due to small access portal, limited visualization or unfavorable angle of approach. A 3-D printed cylindrical chamber 531 filled with artificial CSF with an aperture 533 at the top which holds interchangeable tissue membranes (e.g. porcine dura, bovine pericardium) which emulate the tissue barrier being studied. Chamber pressures are regulated by a calibrated digital manometer with computer interface and servo-infusion pump to produce and record accurate internal pressures, and dual CMOS video cameras are mounted above and below the membrane to assess graft deployment and post-deployment membrane integrity. The membrane is mounted 10 cm below an access platform 535 which accommodates 6 cm long printed cylindrical access portals 537 of 8-22 mm diameter. A standardized 3-15 mm opening in the membrane is created and the tissue repair and sealing applicator assembly tube 539 is inserted through the access portal to the membrane, a graft is deployed below the membrane, secured in place by an external clasp and internal chamber pressures increased corresponding to the pressures inside the human tissue being simulated. The top view with access platform removed (FIG. 32B) illustrates that the access portal can be mounted at angles from 0° to 30° offset from perpendicular to the membrane to simulate angled surgical approaches. Deployment of the graft is visualized on an integrated video screen 541 providing a simulation of endoscopic MIS repair. In FIG. 32C a pressurized cassette 545 is inserted into the spinal canal of a printed model of the human lumbar spine 543 to simulate durotomy in a MIS spinal dural repair.

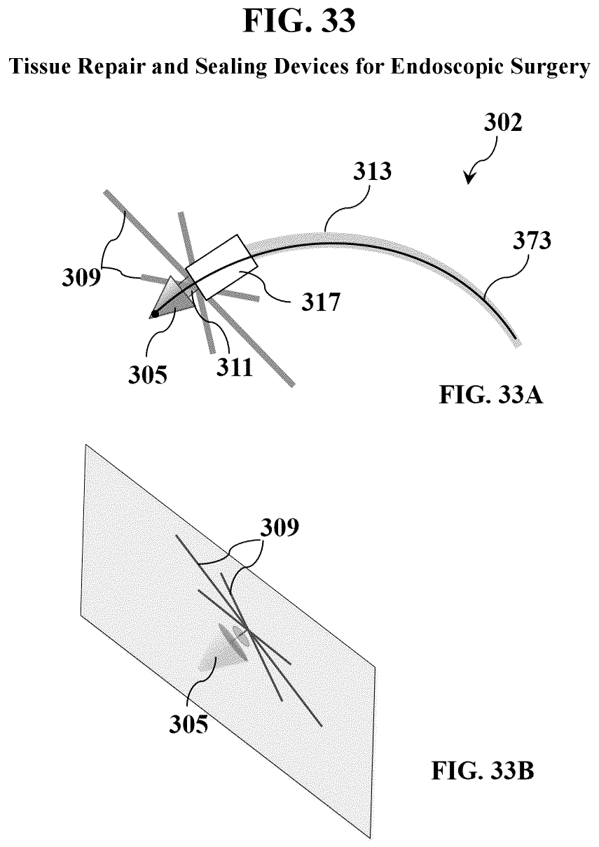

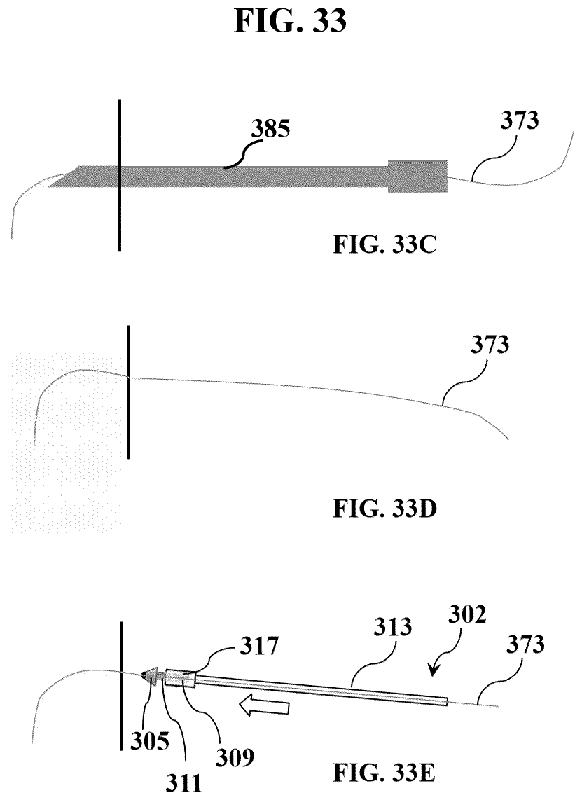

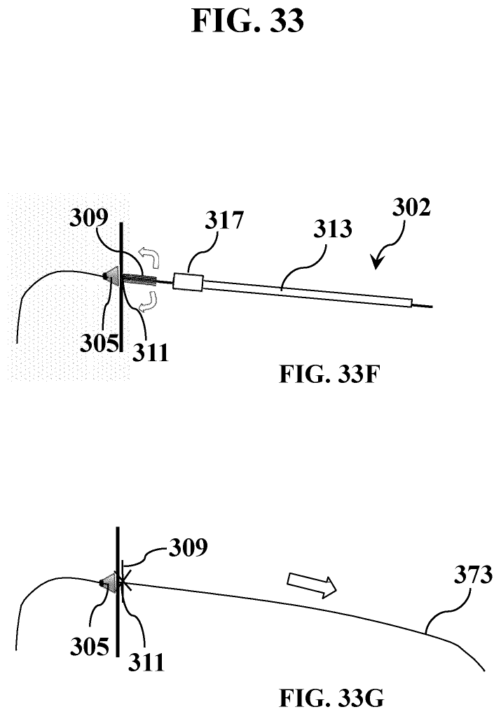

FIG. 33 illustrates an embodiment of the presently disclosed tissue repair and sealing device that is configured for use in surgical procedures (e.g., lumbar punctures and gastrostomies) to occlude a large-bore needle puncture or percutaneous ostomy site. In FIG. 33A is shown a tissue repair and sealing device 302 comprising an applicator assembly 313 having an applicator shaft 373 and a graft-clasp lock cylinder 317 and a detachable graft and clasp assembly having a graft subassembly and a deployable clasp and coupler assembly 311, wherein the graft is a sponge-like conical occluder graft 305, wherein the applicator shaft is fabricated out of a flexible material, and wherein the applicator shaft, central coupler, and graft are configured with a central channel to accommodate a guidewire. In certain aspects, the conical occluder graft is comprised of a bioabsorbable material. In FIG. 33B is shown the deployment of tissue repair and sealing device according to the embodiment presented in FIG. 33A, wherein a conical occluder graft 305 is positioned on an inner tissue surface and the radial struts or spokes 309 of a deployable clasp, or a disc clasp as above are positioned on an outer tissue surface to apply pressure against the outer tissue surface, secure the conical occlude graft, and, thereby, repair a tissue fenestration (i.e., a puncture or ostomy site) and create a pressure-resistant, watertight seal. FIGS. 33C-33G show a tissue repair and sealing device used to repair a puncture site with the use of a guidewire 373. As shown in FIG. 33C, a guidewire is passed through a large-bore needle * that is inserted through a tissue barrier for drainage of fluid. In an alternative aspect of this method, the guidewire may be passed through an indwelling catheter prior to its removal. After removal of the large-bore needle or indwelling catheter, the guidewire remains in place (FIG. 33D). FIG. 33E illustrates the passage of the proximal (external) end of the guidewire 373 through the central channel within the conical occluder graft 305, central coupler 309, and applicator shaft 313. The tissue repair and sealing device is advanced along the guidewire to the puncture site and the conical occluder graft is passed through the puncture hole and positioned against the inner surface of the punctured tissue and the tissue repair and sealing device is deployed by moving the graft-clasp lock cylinder toward the distal end of the applicator shaft to release the deployable clasp 309 and coupler subassembly 311 (FIG. 33F). The deployable clasp is positioned against and applies pressure to the outer tissue surface to secure the conical occlude graft, repair the puncture, and create a pressure-resistant, watertight seal. The applicator assembly is detached from the detachable graft and clasp assembly, which remains at the puncture site, and the applicator assembly is removed by sliding along the guidewire after which the guidewire is then removed (FIG. 33G).

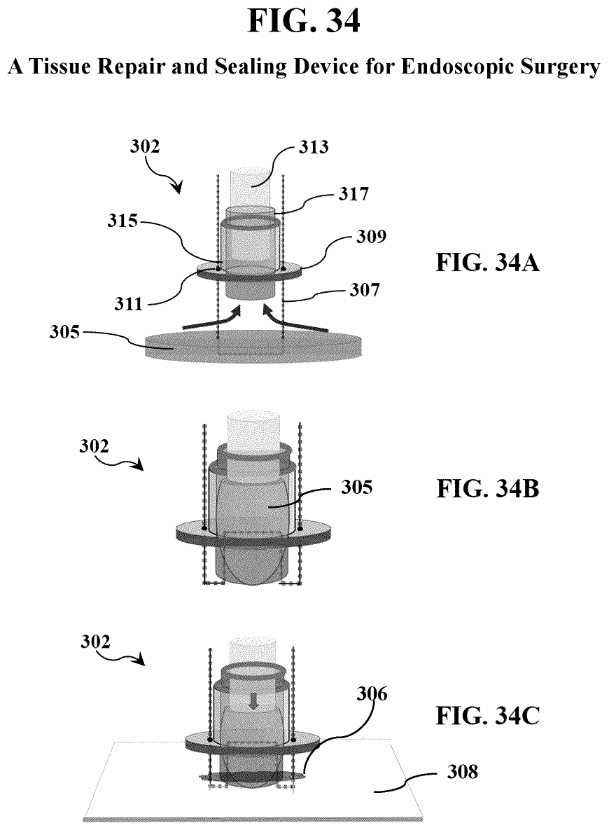

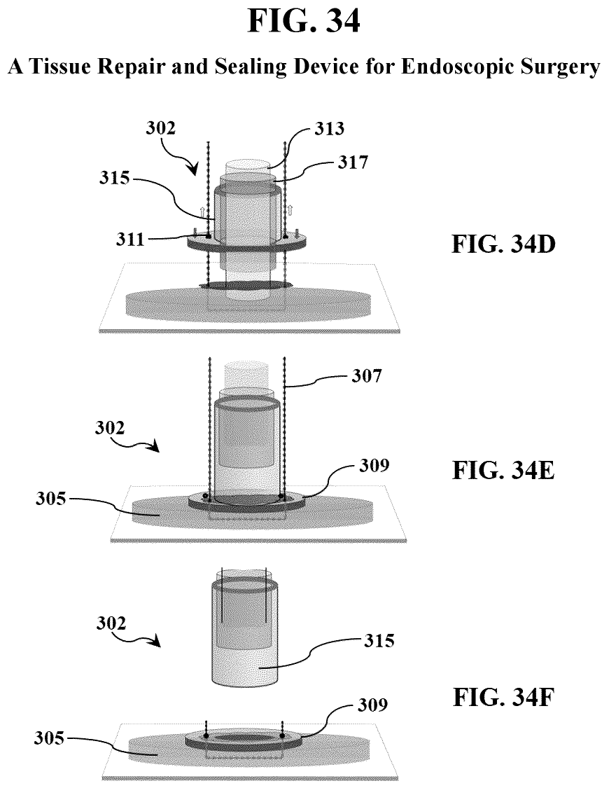

FIGS. 34A-34E show steps in the deployment of a graft-clasp subassembly 302 for use with a narrow delivery tube in an endoscope or large-bore spinal needle comprising a disc-shaped locking clasp 309 and compressible sponge graft 305 according to various tissue repair and seal device embodiments. FIG. 34A shows an external disc clasp which slides over the outer surface of a 1-3 mm delivery tube 317. Barbed suture 307 embedded in the graft is brought through two small holes 311 in the external disc clasp which resist the movement of the clasp relative to the suture. FIG. 34B shows the sponge-like graft with suture 305 compressed inside the delivery tube 317. In FIG. 34C, the delivery tube is positioned at the opening of a fenestration 306 in the dura 308 and a central shaft 313 is moved distally to advance the compressed graft. In FIG. 34D the graft 305 has passed through the dural opening and has expanded in the CSF below the inner surface of the dura. The sliding external disc clasp 309 is advanced proximally (down arrows) along the outer surface of the delivery tube 317 with a cylinder 315 (FIG. 34D) while the barbed suture is retracted proximally (up arrows) holding the graft against the inner dura such that the external disc moves against the outer surface of the dura and the graft-clasp subassembly is held securely by the suture (FIG. 34E), after which the applicator subassembly is detached and removed (FIG. 34F) and the excess suture trimmed.

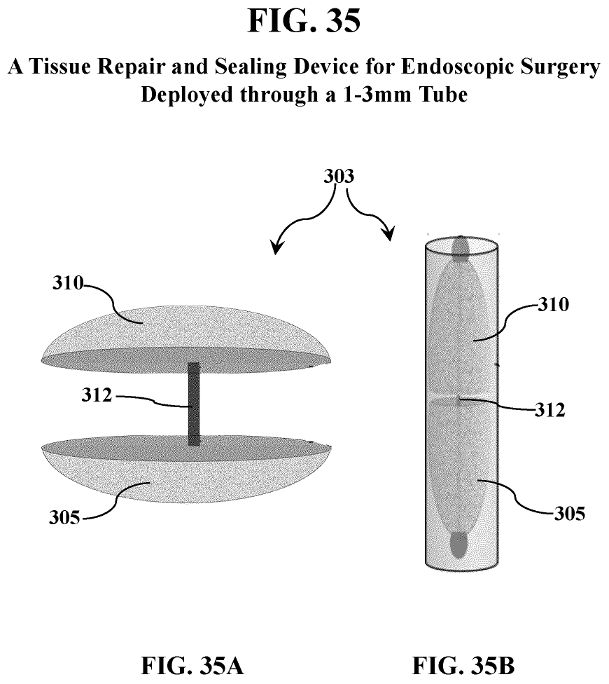

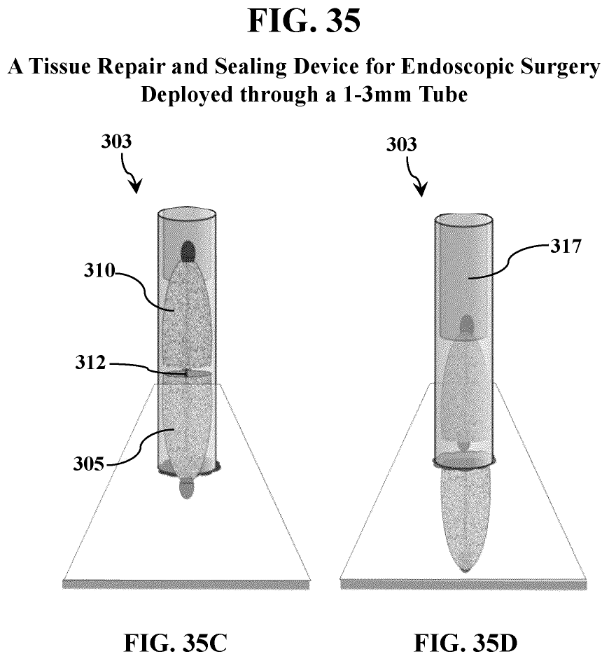

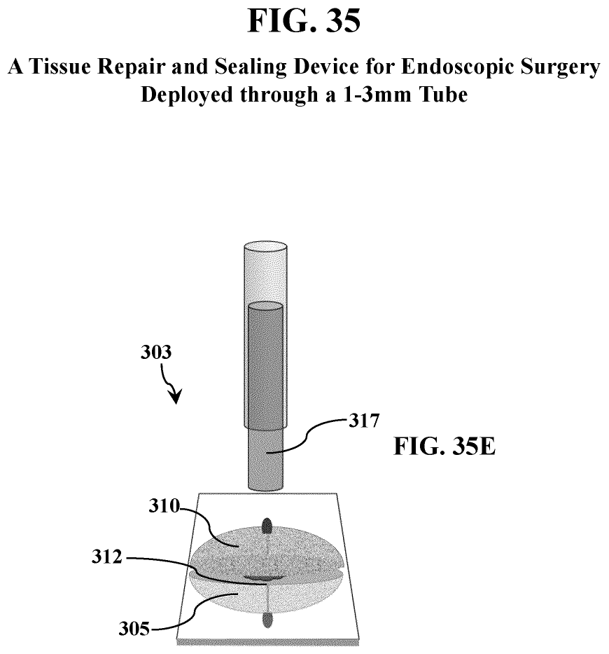

FIG. 35A-E show an alternative configuration of the graft-clasp subassembly 303 for use with an endoscope comprised of internal and external compressible sponges according to various tissue repair and seal device embodiments. FIG. 35A shows the fully deployed graft-clasp subassembly with sponge-like graft or other materials comprising both the internal graft 305 and external clasp 310 connected by a short filament 312. In FIG. 35B, both elements of the graft-clasp subassembly are compressed in a 1-3 mm delivery tube which is positioned at the dural fenestration (FIG. 35C), advanced by a push rod 317 and released sequentially (FIG. 35D) to initially position the graft on the inner surface of the dura, followed by the clasp component to secure the position of the graft on the outer surface (FIG. 35E).

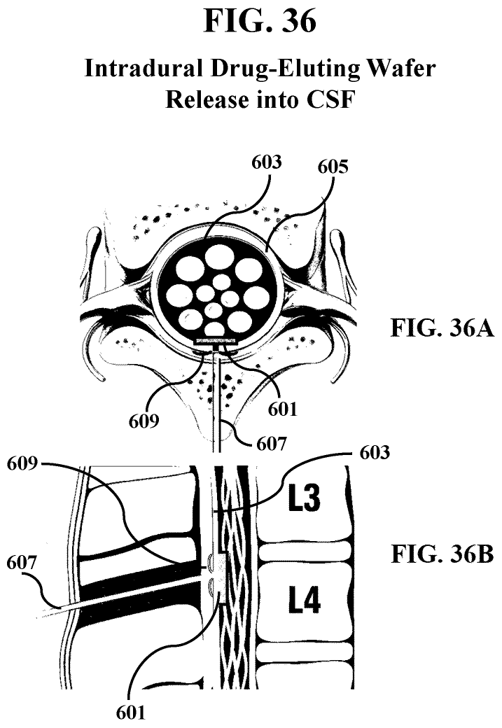

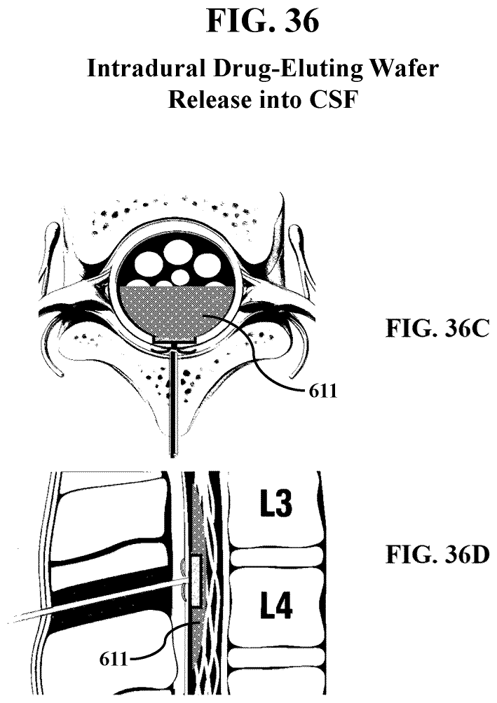

FIG. 36 shows the use of a tissue repair and sealing device comprising an intradural drug-eluting wafer 601 for localized release and administration of a drug to the cerebrospinal fluid 603 (CSF). The wafer is placed inside the dura 605 with the applicator through a large-bore spinal needle 607 and secured with an external clasp 609 (FIG. 36A—coronal view; FIG. 36B—sagittal view. Drug 611 is released from the polymer into the CSF 621 in a continuous regulated manner, where it diffuses throughout the CSF (FIG. 36C-coronal view; FIG. 36D-sagittal view).

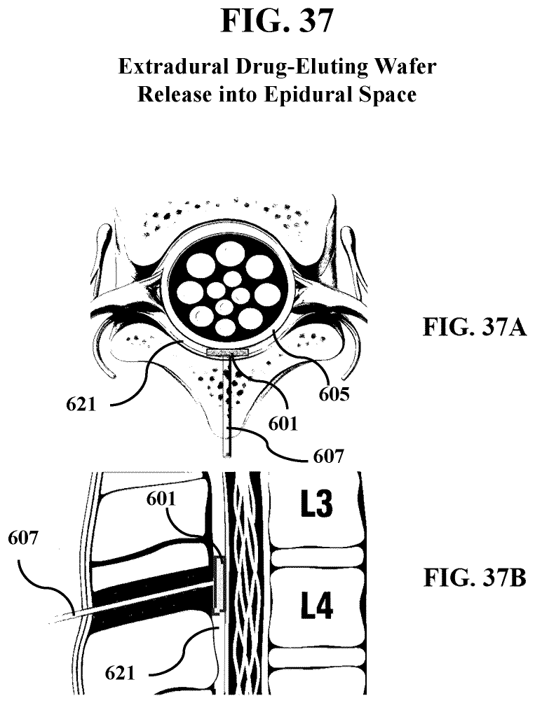

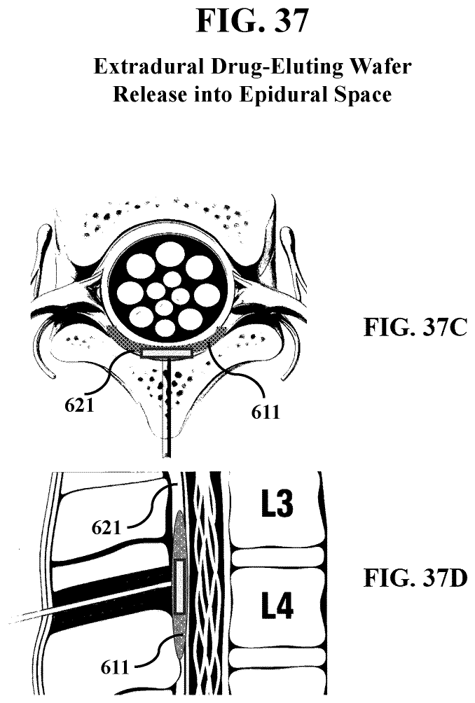

FIGS. 37A-37D show the use of a tissue repair and sealing device comprising an extradural drug-eluting wafer 601 for localized release and administration of a drug to an epidural space 621. The wafer is placed with the applicator immediately outside the dura 605 through a large-bore spinal needle 607 (FIG. 37A—coronal view; FIG. 37B—sagittal view. Drug 611 is released from the polymer in a continuous regulated manner, where it diffuses into the epidural space 621 to provide a local effect on the nerves and spine (e.g. opioid, corticosteroid) at the level of administration (FIG. 37C—coronal view; FIG. 37D—sagittal view.

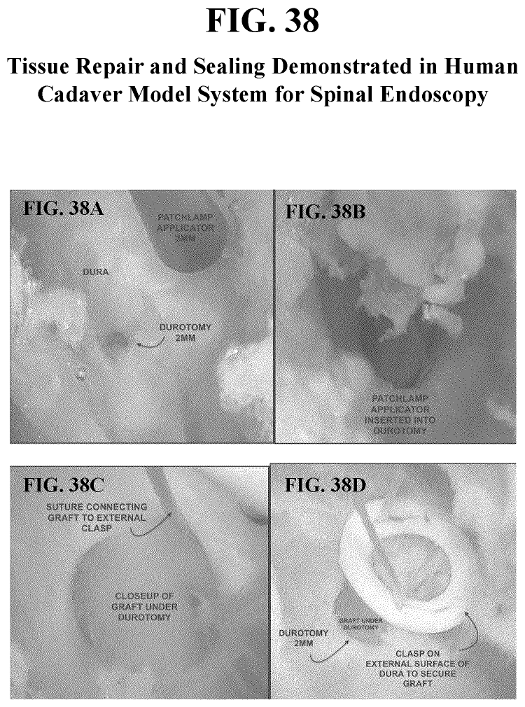

FIG. 38 are photographs demonstrating the use of a tissue repair and sealing device in a human cadaver model system for spinal endoscopy. FIG. 38A shows an endoscopic view of the L4 dura with the 3 mm Tissue repair and sealing device adjacent to a 2 mm durotomy. In FIG. 38B, the graft-clasp lock cylinder has inserted into the entrance of the durotomy. In FIG. 38C, the graft deployed on the inner dural surface is visible through the translucent dura. In FIG. 38D, a disc-shaped bioabsorbable external clasp that was advanced distally on the surface of the applicator assembly is being directed onto the outer surface of the dura to stabilize the internal graft.

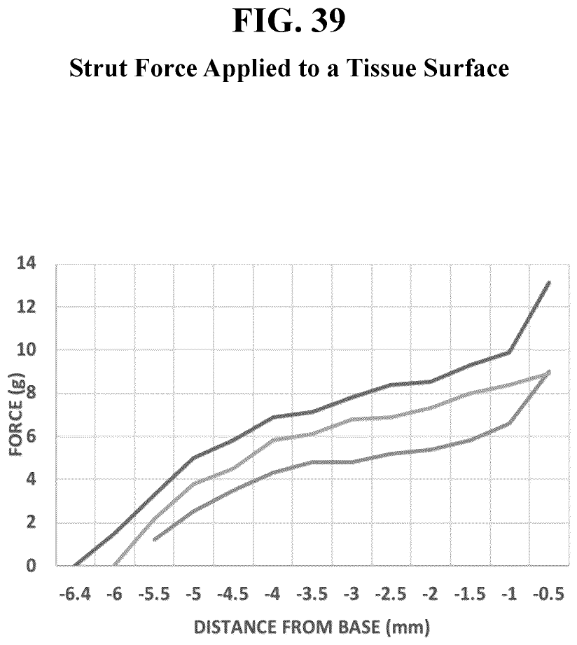

FIG. 39 is a graph of strut force of an external clasp applied to a tissue surface. An external clasp loaded into a tissue repair and sealing device graft-clasp lock cylinder typically deforms the PLA struts to approximately 45-degree angle with approximately 6 mm elevation above the surface. The deformed external clasp component was placed onto a scale, and a precision actuator was used to deflect it downward, and force measurements (grams) were taken at each 0.5 mm increments until the clasp was at its locking position within 0.5 mm of the surface. Force applied to the dura by the external clasp struts increased from 0 to 9-13 gm at their locking position.

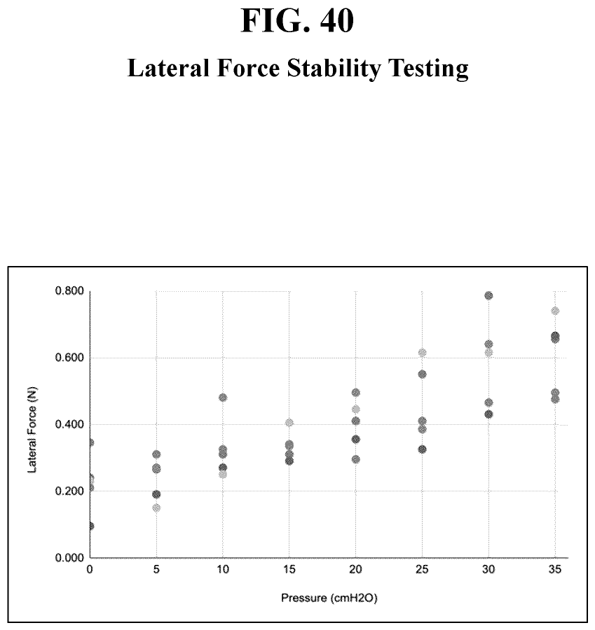

FIG. 40 is a graph of lateral force stability testing of a tissue repair and sealing device graft that is deployed in bovine dura mounted in a pressure chamber over pressures from 0-35 cm H₂O. A wire attached to a coupler base connected to a load cell measured a force of 0.2-0.7 Newtons required to initiate lateral movement which increased with increasing internal pressure of the chamber.

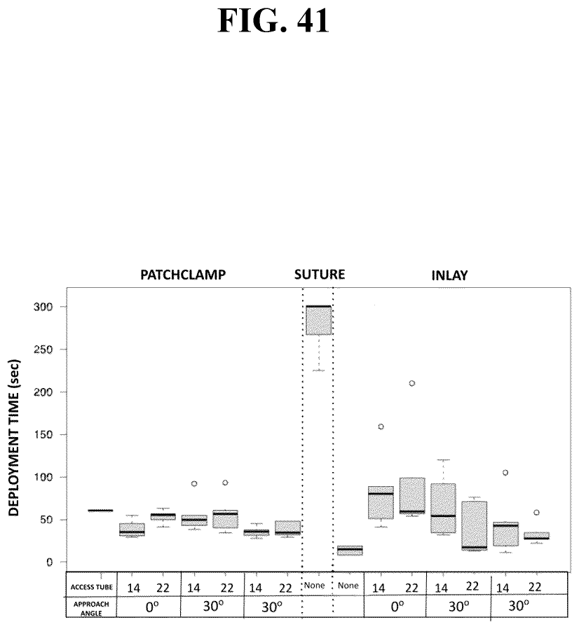

FIG. 41 is a graph representing comparison of the amount of time required for a board-eligible neurosurgeon to repair and seal a durotomy in a constrained access pressure chamber model using three contemporary repair and seal techniques (1) suture closure; (2) placement of an inlay graft; and (3) the tissue repair and sealing device described herein. The device (as presently disclosed) required slightly less repair time (58 sec) compared to inlay graft placement (74 sec), but was substantially better than suture closure (286 sec).

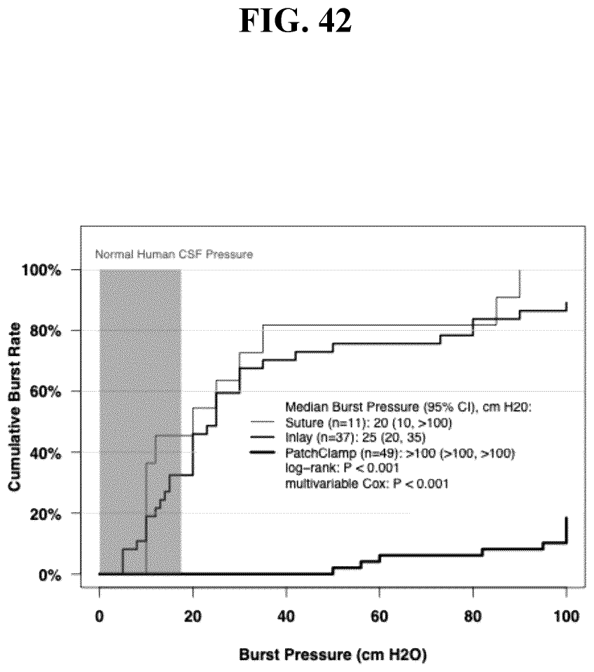

FIG. 42 is a graph comparing measured burst pressure (the internal pressure at which the dural repair burst with unobstructed egress of CSF) for various repair and sealing of durotomy techniques in the pressure chamber. Bursting of the repair occurred at significantly lower pressures for suture repair (20 cm H₂O) and inlay graft (25 cm H₂O) compared to that observed for tissue repair and sealing device described herein (>100 cm H₂O) (p<0.001).

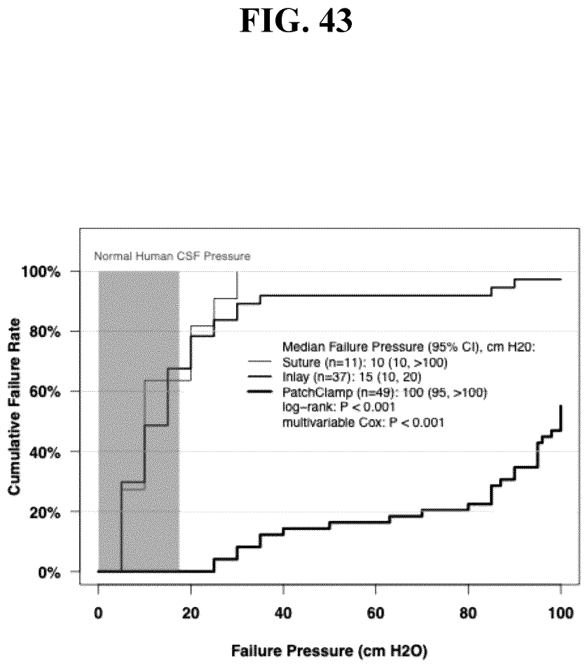

FIG. 43 is a graph comparing measured failure pressure (the internal pressure at which the dural repair first showed leakage of CSF for various repair and sealing of durotomy techniques in the pressure chamber. Failure of the repair occurred at significantly lower pressures for suture repair (10 cm H₂O) and inlay graft (15 cm H₂O) than that observed for tissue repair and sealing device described herein (100 cm H₂O) (p<0.001).

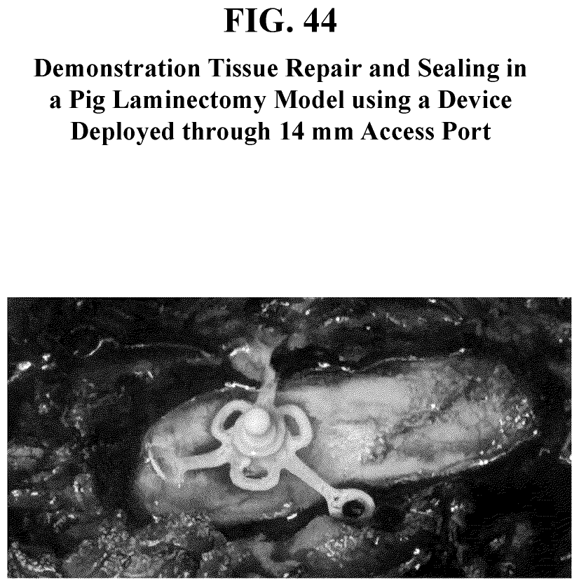

FIG. 44 is a photograph demonstrating the use of the tissue repair and sealing device described herein in a pig laminectomy in vivo model system of durotomy using a graft-clasp subassembly deployed in a MIS procedure through a 14 mm access port. The external clasp and coupler are seen on the external surface of the dura; there was negligible apparent CSF leak after graft placement.

This disclosure will be better understood in view of the following definitions, which are provided for clarification and are not intended to limit the scope of the subject matter that is disclosed herein.

### Definitions

Unless specifically defined otherwise herein, each term used in this disclosure has the same meaning as it would to those having skill in the relevant art.

As used herein, the terms “minimally invasive surgery” and “MIS” are used interchangeably to refer to surgical procedures that avoid the use of open, invasive surgery in favor of closed or local surgery that limit the size of incisions to lessen wound healing time, associated pain, and risk of infection as compared to traditional “non-MIS” procedures. MIS procedures, such as such as endoscopy, laparoscopy, arthroscopy, involve the use of laparoscopic devices and remote-controlled manipulation of instruments with indirect observation of the surgical field through an endoscope or similar device, such as neuroendoscopy. MIS procedures also include the use of hypodermic injection, and air-pressure injection subdermal implants, refractive surgery, percutaneous surgery, cryosurgery, microsurgery, keyhole surgery, endovascular surgery using interventional radiology (such as angioplasty), coronary catheterization, permanent placement of spinal and brain electrodes, stereotactic surgery, the Nuss procedure, radioactivity-based medical imaging methods, such as gamma camera, positron emission tomography and SPECT (single photon emission tomography). Related procedures are image-guided surgery, and robot-assisted surgery.

As used herein, the term “tissue barrier” refers to a layer of tissue in the body that separates two body compartments. “Tissue barriers” function in vivo as both protective shields and gate keepers between different compartments (e.g., blood and tissue) and are created by specialized membrane-associated proteins, located at the lateral plasma membrane of epithelial and endothelial cells. By sealing the paracellular space, such barriers impede the free diffusion of solutes and molecules across epithelial and endothelial monolayers, thereby creating an organ-specific homeostatic milieu. Tissue barriers include tissues that comprise the meninges, dura of the nervous system, abdominal wall, muscle fascia, blood vessels, esophagus, oropharynx, stomach, small and large intestine, rectum, trachea, bronchus, heart, bladder, ureter, urethra, uterus, peritoneum, pleura, fallopian cylinder, sclera of the eye, synovium, tympanic membrane or the capsule of a solid organ (e.g., kidney, liver, and pancreas), fluid or space contained by the tissue barrier (blood, cerebrospinal fluid, gastrointestinal contents, pleural cavity, peritoneal cavity, vitreous humor, inner ear, fallopian cylinder or joint space).

As used herein, the term “meninges” refers, collectively, to the three membranes (the dura mater, arachnoid mater, and pia mater) that line the skull and vertebral canal, enclose the brain and spinal cord, and protect the central nervous system. “Meningitis” is the inflammation of the meninges, which is typically caused by an infectious agent.

As used herein, the terms “dura” and “dura mater” are used interchangeably and refer to the outermost (i.e. closest to the skull and vertebrae) of the three layers of membrane called the meninges (i.e. the meningeal layers) that are made of dense irregular connective tissue. “Dura mater” (a/k/a “pachymeninx”) is derived primarily from the neural crest cell population, with postnatal contributions of the paraxial mesoderm. “Dura mater” protects the central nervous system by surrounding the brain and the spinal cord and along with the arachnoid mater provides a watertight enclosure for the cerebrospinal fluid (CSF). As used herein, the terms “arachnoid mater” and “pia mater” refer to the two inner meningeal layers that are enveloped by the “dura” or “dura mater.” The “arachnoid mater” is interposed between the much thicker “dura mater” and the deeper “pia mater.” The “arachnoid mater” is separated from the “pia mater” by the subarachnoid space and is responsible for retaining cerebrospinal fluid (“CSF”) within the subarachnoid space (“SAS”), The “pia mater” is a thin, water permeable, fibrous tissue that permits blood vessels to pass through and nourish the brain. The “arachnoid mater” and “pia mater” are known collectively as the “leptomeninges” and have complex functions as barriers and facilitators for the movement of fluid, solutes and cells at the surface of the CNS and of fluid and solutes within the CNS parenchyma. Reviewed by Weller et al., Acta Neuropathologica 135:363-385 (2018). Both the “arachnoid mater” and “pia mater” derive from the neural crest.

As used herein, the term “fenestration” refers to an opening in a body tissue barrier, such as a cut, tear, puncture, defect, or other breach. A fenestration may be spontaneous (e.g., a cerebrospinal leak from a congenital defect); secondary (e.g., a tissue barrier that is compromised by a tumor or infection); planned (e.g., an incision or puncture of a blood vessel, dura mater, or outer wall of a body organ); or unplanned (e.g., inadvertent durotomy, intestinal breach, or laceration of the wall of a body organ during a surgical procedure). A fenestration in a tissue barrier usually requires repair and sealing to prevent serious complications (e.g., infection, bleeding, and wound breakdown). However, for MIS procedures, the combination of restricted working space and access vectors, limitation of vision, and the nature and consistency of the fenestrated tissue barrier, significantly limit direct repair and sealing by traditional methods (e.g., suturing or stapling).

As used herein, the terms “durotomy,” “unintended durotomy,” and “incidental durotomy” refer to an unintended tear of the dura mater (dural tear) that commonly occurs during MIS procedures performed on the spine (e.g., lumbar microdiscectomy). The complexity of spinal MIS procedures contributes to the incidence of“durotomy.” Durotomies require immediate repair and watertight sealing to prevent post-surgical complications, including leakage of cerebrospinal fluid with subsequent meningitis, or the accumulation of air in the spinal canal (i.e. pneumorachis, aerorachia, or epidural emphysema), most commonly within the extradural or subarachnoid space with disruption of the surrounding dura mater.

As used herein, the term “graft” refers, generally, to tissues, membranes, meshes, matrices, and the like that exhibit suitable biophysical properties and are of the appropriate size, shape, and other dimensions for adhering to inner tissue surfaces, repairing tissue fenestrations, and creating pressure-resistant, watertight seals. “Grafts” may derive from natural sources such as animal organ tissues and tissue barriers and include tissues from a donor that exhibit a defined genetic relationship to tissues from a recipient such as, for example, autografts (tissue obtained from patient), isografts (tissue obtained from a monozygotic twin), allografts (tissue obtained from another person), or xenografts (tissue obtained from a non-human animal species). Grafts from such natural sources may be autologous, homologous, or heterologous and may incorporate one or more synthetic material.

As used herein, the term “drug eluting graft” refers to graft materials that incorporate a drug eluting matrix to provide controlled focal drug release. Han and Lelkes, Focal Controlled Drug Delivery, Advances in Delivery Science and Technology (Springer, Boston, 2014).

As used herein, the term “non-resorbable” refers to materials that are not broken down and absorbed by the body, and thus are intended for long-term, structural applications. “Non-resorbable” materials include implantable polymers, such as polyethylene and polyketones (PEEK), phase pure R Tricalcium phosphate (TCP), and hydroxyapatite (HA).

As used herein, the term “bioresorbable” refers to materials that are broken down and absorbed by the body, and thus do not need to be removed manually. Bioabsorbable materials include polymers including biopolymers, and copolymers thereof, such as polylactide (PLA), polyglycolide (PGA), polylactide-co-D, L lactide (PDLLA), polylactide-co-glycolide (PLGA), polylactide-co-caprolactone (PLCL), polycaprolactone (PCL), polydioxanone (PDO), polylactide-co-trimethylene carbonate (PL-TMC) which can be customized to meet mechanical performance parameters, biocompatibility, and resorption rates. Within certain aspects, the tissue repair and sealing devices disclosed herein comprise a graft that is either directly incorporated (i.e., “integrated”) into the detachable graft and clasp assembly or is substituted at the time of surgery using the form rings as described herein. Tissue repair and sealing devices according to this disclosure may employ a fixed central coupler that maintains the detachable graft and clasp assembly in a perpendicular orientation relative to the applicator assembly or may employ an adjustable central coupler that permits movement of the detachable graft and clasp assembly relative to applicator assembly for use in enhancing the visibility of the tissue fenestration and nearby structures.

As used herein, the term “graft” refers, generally, to tissues, membranes, meshes, matrices, and the like that exhibit suitable biophysical properties and are of the appropriate size, shape, and other dimensions for adhering to inner tissue surfaces, repairing tissue fenestrations, and creating pressure-resistant, watertight seals. “Grafts” may derive from natural sources such as animal organ tissues and tissue barriers and include tissues from a donor that exhibit a defined genetic relationship to tissues from a recipient such as, for example, autografts (tissue obtained from patient), isografts (tissue obtained from a monozygotic twin), allografts (tissue obtained from another person), or xenografts (tissue obtained from a non-human animal species). Grafts from such natural sources may be autologous, homologous, or heterologous and may incorporate one or more synthetic material.

As used herein, the term “synthetic mesh” refers to a graft made from non-biologic materials including poly(ethylene terephthalate) (a/k/a Dacron®) or expanded polytetrafluorethylene (ePTFE, Goretex®) and are described in Patera and Schoen, Biomaterials Science pp. 470-494 (Elsevier Academic Press, San DEieto, CA (2004)). “Synthetic meshes” are often permanent in nature, do not undergo bioresorption, and are associated with chronic inflammation and foreign body reactions, firmness and fibrosis, and infection. Schmatz, Cureus 10(1):e2127 (2018) provides a report on surgical experience with an synthetic, bioabsorbable graft material that has received FDA approval.

As used herein, the term “biologic mesh” refers to a graft that is derived from animal tissue, typically human or porcine dermis, and processed to an acellular, porous extracellular matrix scaffold of collagen and elastin. Often a “biological mesh” contains growth factors from the source tissue, which attract endothelial cells and fibroblasts, which release additional chemoattractants that signal the migration of other structural cells. The three-dimensional nature and porosity of “biological meshes” allow cells (mainly fibroblasts and inflammatory cells) to enter the mesh and adhere and undergo a cycle of remodeling consisting of degradation of the biologic mesh and regeneration of the collagen scaffold with host tissue. The balance of this degradation and rebuilding process, and the speed with which it occurs, influences the ultimate strength and structure of the repaired tissue. “Biologic meshes” can be crosslinked to increase graft firmness, although greater cell infiltration is typically observed with biologic meshes that are not crosslinked. Crosslinking can also prevent collagen breakdown and inhibit macrophage migration, which poses and increased risk of infection.

As used herein, the term “dural substitute” refers to a graft, either synthetic or biologic, for use in sealing dural tissue fenestrations by absorbing and integrating onto the patient's tissue to prevent CSF leaks and to allow openings in the dura to heal after surgery. “Dural substitutes” that may be advantageously employed in the tissue repair and sealing devices disclosed herein include the Duraform® dural graft implant (Natus, Medical Inc., Middleton, WI), which is a collagen-based biocompatible material with high tensile strength that is manufactured from processed bovine tendons; the Biodesign® Dural Graft and Duraplasty graft (Cook Medical, Bloomington, IN), which employ a natural extracellular matrix (ECM) derived from porcine small intestinal submucosa (SIS); DuraGen® Matrix (Integra LifeSciences, Princeton, NJ), which is a collagen matrix; DuraMatrix® (Stryker, Kalamazoo, MI), which is a collagen matrix derived from bovine tissues; Cerafix dural Graft®, which is a synthetic, resorbable material; PRECLUDE Dura Substitute®, which is an inert elastomeric fluropolymer (ePTFE): Lyoplant Onlay Graft®, which is an absorbable collagen bilayer: Neuro-Patch Dural Graft®, which is microporous fleece; SEAMDURA®, which is a copolymeric film layered with PGA; and Durepair™ Regeneration Matrix (Medtronic, Minneapolis, MN), which is a non-synthetic collagen matrix derived from Type III fetal bovine tissue.

As used herein, the term “drug eluting graft” refers to graft materials that incorporate a drug eluting matrix to provide controlled focal drug release. Han and Leikes, Focal Controlled Drug Delivery, Advances in Delivery Science and Technology (Springer, Boston, 2014.

As used herein, the term “non-resorbable” refers to materials that are not broken down and absorbed by the body, and thus are intended for long-term, structural applications. “non-resorbable” materials include implantable polymers, such as polyethylene and polyketones (PEEK), phase pure β Tricalcium phosphate (TCP), and hydroxyapatite (HA).

As used herein, the terms “passivated metal” or “passivated metal alloy” refer to metals and metal alloys that are resistant to corrosion and exhibit enhanced biocompatibility as compared to the native metal or metal alloy. Passivation may be achieved by applying an outer layer of shield material as a microcoating on the exposed surface of the metal or metal alloy.

Words and phrases using the singular or plural number also include the plural and singular number, respectively. For example, terms such as “a” or “an” and phrases such as “at least one” and “one or more” include both the singular and the plural. Terms that are intended to be “open” (including, for example, the words “comprise,” “comprising,” “include,” “including,” “have,” and “having,” and the like) are to be construed in an inclusive sense as opposed to an exclusive or exhaustive sense. That is, the term “including” should be interpreted as “including but not limited to,” the term “includes” should be interpreted as “includes but is not limited to,” the term “having” should be interpreted as “having at least.”

The use of the term “or” in the claims is used to mean “and/or” unless explicitly indicated to refer to alternatives only or the alternatives are mutually exclusive, although the disclosure supports a definition that refers to only alternatives and “and/or.”

Additionally, the terms “herein,” “above,” and “below,” and words of similar import, when used in this application, shall refer to this application as a whole and not to any particular portion of the application.

It will be further understood that where features or aspects of the disclosure are described in terms of Markush groups, the disclosure is also intended to be described in terms of any individual member or subgroup of members of the Markush group. Similarly, all ranges disclosed herein also encompass all possible sub-ranges and combinations of sub-ranges and that language such as “between,” “up to,” “at least,” “greater than,” “less than,” and the like include the number recited in the range and includes each individual member.

The practice of the present disclosure will employ conventional techniques and methodologies that are in common use in the field of medicine, in particular in conjunction with minimally invasive surgical (MIS) procedures and non-minimally invasive surgical (non-MIS) procedures. Such techniques and methodologies are explained fully in treatises on surgical procedures as well as the medical, scientific, and patent literature. See, e.g., Hunter and Spight, “Atlas of Minimally Invasive Surgical Operations” (McGraw-Hill Education, Inc., 2018); Jones and Schwaltzberg, “Operative Endoscopic and Minimally Invasive Surgery” (CRC Press, 2019); and Nahai, “The Art of Aesthetic Surgery” (2^{nd} Ed., Thieme, 2010).

All references cited herein, whether supra or infra, including, but not limited to, patents, patent applications, and patent publications, whether U.S., PCT, or non-U.S. foreign, and all technical, medical, and/or scientific publications are hereby incorporated by reference in their entirety.

### Tissue Repair and Sealing Devices and Uses Thereof

Within certain embodiments, the present disclosure provides tissue repair and sealing devices and methods for the use of those tissue repair and sealing devices, which uses comprise: (1) selecting a releasable graft-clasp subassembly, as disclosed herein, (2) attaching the releasable graft and clasp assembly to an applicator subassembly comprising a graft-clasp positioning cylinder, a graft-clasp release cylinder, and a graft-clasp lock cylinder that are nested concentrically, (3) folding the clasp and inserting into the outer graft-clasp lock cylinder (4) positioning the graft on an inner tissue surface and positioning the deployable clasp on an outer tissue surface by deploying the inner graft-clasp positioning cylinder, (5) securing the graft to the inner tissue surface by deploying the graft-clasp lock cylinder to lock the clasp into the graft-clasp coupler, (8) releasing the deployable clasp onto the outer surface by deploying the central graft-clasp release cylinder to, thereby, repair a tissue fenestration and create a pressure-resistant, watertight seal.

In operation, graft and coupler members can be assembled together separately from a clasp member, which comprises radial external struts emanating from a central ring. In an exemplary embodiment, the coupler has a small coupling peg on the external side of the graft, which attaches reversibly to the graft-clasp positioning cylinder and has a groove for locking the clasp ring tightly against the outer tissue surface to create a pressure-resistant, watertight seal.

The graft and clasp are folded and contained within the graft-clasp lock cylinder. It is released to expand and be positioned beneath the tissue fenestration. In certain aspects of embodiments for open (non-MIS) surgical procedures having large tissue fenestrations, the graft is left expanded at the time of application rather than folded into the graft-clasp lock cylinder. After the graft is deployed under the tissue fenestration, the radial struts are released from the graft-clasp lock cylinder which, when deployed, contacts the ring/bushing of the deployable clasp and slides the ring/bushing into a groove in the graft-clasp coupler to lock the clasp in place.

In certain aspects of these embodiments, the tissue repair and sealing devices disclosed herein employ a detachable graft-clasp subassembly comprising a deployable clasp and graft connected via a central coupler, with the deployable clasp having a plurality of radial struts or spokes that emanate from the central coupler at or near the geometric center of the detachable graft and clasp assembly. The graft-clasp subassembly reversibly attaches via the central coupler to the applicator subassembly at the distal end of the graft-clasp positioning cylinder and is deployed by the actuating rocker 135 to secure the graft to the inner tissue surface and the clasp to the outer tissue surface to repair a tissue fenestration and create a pressure-resistant, watertight seal.

In operation, tissue repair and sealing devices disclosed herein permit the positioning of (1) a graft subassembly on an inner tissue surface and (2) a deployable clasp and coupler subassembly on an outer tissue surface. Prior to use, a detachable graft and clasp assembly is attached via a coupler to a graft-clasp positioning cylinder applicator assembly at the distal end of a graft-clasp positioning cylinder. The radial spokes or struts of a deployable clasp are folded toward the folded graft such that both the clasp and the graft are folded toward the distal end of the graft-clasp positioning cylinder and within the graft-clasp lock cylinder.

Using the applicator subassembly, the graft-clasp subassembly is inserted through a tissue fenestration and positioned on an inner tissue surface while the deployable clasp and coupler assembly remains outside of the fenestrated tissue. The tissue repair and sealing devices are deployed by actuating the graft-clasp positioning cylinder to position the graft and clasp, locking the clasp against the outer tissue surface by actuating the graft-clasp lock cylinder, and releasing the graft-clasp subassembly from the applicator subassembly by actuating the graft-clasp release mechanism to rapidly repair a tissue fenestration and reliably create a pressure-resistant, watertight seal.

Additional modifications of the tissue repair and sealing devices are described herein that address specific technical problems encountered in MIS surgery. These include (1) variations in the size and shape of graft subassemblies and deployable clasp and coupler subassemblies, (2) variations in the materials used for the graft subassemblies and deployable clasp and coupler subassemblies, (3) rotation of the coupling component such that the graft can be oriented such that it is not perpendicular to the graft-clasp positioning cylinder, thereby improving line-of-sight visualization of the fenestration during insertion of the graft, (4) configurations that permit the use of tissue repair and sealing devices in endoscopic or percutaneous procedures (e.g., modifications of the graft-clasp subassembly or the use of conical graft elements and flexible applicator assemblies having a channel for accommodating a guide wire), and (5) the incorporation of drug-eluting matrix materials in place of or in combination with the graft component to provide the continuous drug delivery at the site of application.

Exemplified herein are tissue repair and sealing devices that comprise a deployable clasp having a plurality of flexible spokes or struts that emanate radially from a coupler wherein the deployable clasp exhibits suitable biophysical properties, size, shape, and dimensions to secure a graft that is positioned on an inner tissue surface and a clasp that is positioned on an outer tissue surface and to, thereby, repair a tissue fenestration and create a pressure-resistant, watertight seal.

In operation, tissue repair and sealing devices disclosed herein permit the positioning of (1) a graft on an inner tissue surface and (2) a deployable clasp and coupler on an outer tissue surface. Prior to use, a detachable graft-clasp subassembly is attached via a coupler to an applicator subassembly at the distal end of a graft-clasp positioning cylinder. The radial spokes or struts of a deployable clasp and graft are both folded toward the distal end of the applicator subassembly.

Using the applicator assembly, the graft is positioned inserted through a tissue fenestration and positioned on an inner tissue surface while the deployable clasp remains outside of the fenestrated tissue. The tissue repair and sealing devices are deployed by (1) actuating the graft-clasp positioning cylinder to position the grasp on an inner tissue surface and clasp on an outer tissue surface, (2) moving the graft-clasp lock cylinder toward the distal end of the graft-clasp positioning cylinder to release the deployable clasp, which permits the deployable clasp to unfold, apply pressure to the outer tissue surface, secure the graft subassembly to the inner tissue surface and, thereby, to rapidly repair a tissue fenestration and reliably create a pressure-resistant, watertight seal.

Exemplified herein are deployable devices that comprise a deployable clasp having a plurality of flexible spokes or struts that emanate radially from the coupler wherein the deployable clasp exhibits suitable biophysical properties, size, shape, and dimensions to secure a graft that is positioned on an inner tissue surface and a clasp that is positioned on an outer tissue surface and to, thereby, repair a tissue fenestration and create a pressure-resistant, watertight seal.

Exemplified herein are deployable devices that comprise a deployable clasp having a plurality of flexible spokes or struts that emanate radially from the coupler wherein the deployable clasp exhibits suitable biophysical properties, size, shape, and dimensions to secure a graft that is positioned on an inner tissue surface and a clasp that is positioned on an outer tissue surface to, thereby, repair a tissue fenestration and create a pressure-resistant, watertight seal.

In certain deployable devices, the graft comprises a flexible, bendable, firm, and compressible material. In some aspects of these embodiments the graft exhibits shape memory and superelasticity characteristics. Grafts according to these embodiments, when used in combination with a deployable clasp, are suitably employed for the repair of tissue fenestrations and creation of pressure-resistant, watertight seals when the graft is positioned on an inner tissue surface and secured with a deployable clasp on an outer tissue surface.

The devices and methods described herein may be applied to direct visual, percutaneous, or endoscopic repair and sealing of multiple tissues in the body. In addition, the device and methods described herein may be modified to address problems specific to the nature, condition and surgical exposure of the fenestrated tissue, including variations of the clasp material and orientation, a component to enable the intraoperative substitution of different graft materials, variations in the size and shape of the graft-clasp unit, and percutaneous or endoscopic repair and sealing of punctures or ostomies using a flexible applicator with or without guide wire.

Prior to using the repair and sealing device, the deployable clasp is folded and inserted into the slidably attached graft-clasp lock cylinder. Upon positioning of the graft on the inner tissue surface of the fenestrated tissue and the clasp on the outer tissue surface, the device is deployed by sliding the graft-clasp lock cylinder along the graft-clasp positioning cylinder toward its distal end. When the device is deployed, the clasp struts or spokes are released from the graft-clasp lock cylinder and contact the outer tissue surface of the fenestrated tissue, thereby securing the graft and clasp in place to repair the tissue fenestration and create a watertight seal.

Additional modifications of the tissue repair and sealing device are described herein which address specific technical problems encountered in MIS surgery. These include variations in graft-clasp unit size and shape, a rotational attachment at the coupling device to enable positioning of the graft relative to the applicator to improve line of vision and access, variations in the strut materials and configuration, use of a flexible applicator and guide wire channel for use of the device in endoscopic or percutaneous procedures, and the incorporation of drug-eluting matrix materials in the graft component to provide continuous drug delivery at the site of application.

Within further aspects, the tissue repair and sealing devices disclosed herein utilize a graft-clasp subassembly wherein the graft comprises a material that is selected from the group consisting of an autograft, an isograft, an allograft, and a xenograft. In related aspects grafts are derived from an animal tissue selected from the group consisting of a human tissue, a bovine tissue, and a porcine tissue and include, for example, an animal tissue is selected from the group consisting dermis, pericardium, and intestine.

In still further aspects, the graft comprises a material that is derived from an animal tissue, such as an animal tissue that is selected from the group consisting a human tissue, a bovine tissue, and a porcine tissue, including an animal tissue that is selected from the group consisting of dermis, pericardium, and intestine. Grafts according to these embodiments may comprise one or more of the following: (1) an acellular, porous extracellular matrix scaffold; (2) collagen; (3) elastin; and (4) a growth factor. In some aspects, grafts according to these embodiments comprise a mesh having a porosity that is sufficient to allow cells to enter, adhere, and undergo a cycle of remodeling.

Within yet other aspects, the tissue repair and sealing devices disclosed herein utilize a graft comprising an acellular, porous extracellular matrix scaffold of collagen, elastin, and, optionally, a growth factor. Such grafts optionally comprise a mesh having a porosity that is sufficient to allow cells to enter, adhere, and undergo a cycle of remodeling. Grafts may additionally comprise a drug eluting matrix.

Within other aspects, the tissue repair and sealing devices disclosed herein utilize a detachable graft and clasp assembly in which one or more elements of the graft subassembly and/or the deployable clasp and coupler subassembly comprise comprises a biocompatible, non-ferromagnetic, passivated metal or metal alloy wire that exhibits shape memory and superelasticity characteristics that permit the folding of said metal or metal alloy while retaining the capacity to unfold to a pre-folded state. Suitable biocompatible, non-ferromagnetic, passivated metal or metal alloy wires include wires comprising a metal or metal alloy that is selected from the group consisting of pure titanium; a titanium-based alloy; a cobalt-based alloy; a platinum-based alloy; and a molybdenum, tungsten, and tantalum alloy. Suitable metal or metal alloy having shape memory and superelasticity characteristics that are enhanced at elevated temperature include, for example, a nickel-titanium alloy (Nitinol) and a niobium-titanium alloy.

TABLE 2
Broad Suitability of Tissue Repair and Seal Devices for Use in Open and MIS
Surgical Procedures Across a Wide Range of Tissues, Diseases, and Conditions

| Tissue | | Open | MIS | Disease or Condition |
|---|---|---|---|---|
| Cranial Dura | Large Defect with Graft | X | | Tumor |
| | | | | Chiari repair |
| | | | | Trauma Infection |
| | | | | Augmentation |
| | Incision - Large | X | | Tumor |
| | | | | Epilepsy |
| | | | | Hemorrhage |
| | Incision - Burr Hole | X | X | Ventriculostomy |
| | | | | Electrode placement |
| | | | | Hemorrhage |
| | Pituitary | | | Tumor |
| | | | | Apoplexy |
| | Anterior Skull Base | X | X | Tumor |
| | | | | CSF Leak |
| | Posterior Skull Base | X | X | Tumor |
| | | | | CSF Leak |
| Spinal Dura | Large Defect with Graft | X | | Tumor |
| | | | | Augmentation for cord |
| | | | | decompression |
| | Planned Durotomy - Large | X | | Tumor |
| | | | | Infection |
| | Inadvertent Durotomy | X | X | Many procedures |
| | Lumbar Puncture | | X | Lumbar puncture |
| Cranial Sinus | Sinus Wall | | X | Defect from tumor |
| | | | | Infection |
| | Inadvertent | | | CSF leak after endonasal |
| | | | | surgery |
| Oral | | X | | Post molar extraction |
| Maxillofacial | | | | |
| Cardiovascular | Heart | | X | PFO |
| | Artery | X | X | Arteriotomy |
| | Vein | X | X | Venipunture or Trauma |
| Gastrointestinal | Abdominal Wall | X | X | Gastrostomy |
| | | | | Hernia |
| | Peritoneum | X | X | Abdominal surgery |
| | | | | Trauma |
| | Intestine | | | Tumor |
| | | | | Inflammation |
| | | | | Trauma (Inadvertent) |
| | Gall Bladder | X | X | Cholecystectomy |
| | Liver/Spleen Capsule | X | X | Trauma |
| | Appendix | X | | Appendectomy |
| | Esophagus | X | X | Tumor |
| | | | | Radiation |
| | | | | Surgery (Inadvertent) |
| | Stomach | X | X | Gastrostomy |
| Genito Urinary | Kidney Capsule | | X | Trauma |
| | Kidney Calyx | | X | Kidney stones |
| | | | | Infection |
| | Ureter | | X | Kidney Stones |
| | | | | Infection |
| | | | | Surgery (Inadvertent) |
| | Bladder | X | X | Tumor |
| | | | | Infection |
| | | | | Surgery (Inadvertent) |
| | Urethra | X | | Trauma |
| | Seminal Vesicle | | X | Inadvertent Vasectomy |
| Otologic | Eardrum | X | X | Infection |
| | | | | Surgery |
| Respiratory | Oropharynx | X | X | Tumor |
| | | | | Radiation |
| | Trachea/Bronchus | X | X | Tumor |
| | | | | Radiation |
| | Pleura | | X | Chest Cylinder |
| | Chest Wall | X | | Thoracotomy or Trauma |
| Orthopedic | Synovium | | X | Arthroscopy |
| Ophthalmologic | Sclera | | X | Vitrectomy |

Another application of the inventions described herein for dural closure may be for open (non-MIS) procedures of the cranium and spine, wherein a planned incision in the dura is made to expose the underlying neural structures during craniotomy or laminectomy procedures. Suture closure of the dural incision is generally employed, but is time-consuming and often not watertight. Multiple grafts of variable size and shape may be combined for closure of a long dural incision of duroplasty. Additionally, the device may utilize the fixed perpendicular orientation of the graft-clasp unit on the applicator shaft or an adjustable coupling device to enable rotation of the graft-clasp unit to facilitate visualization of the fenestration and nearby structures. Modifications of the applicator assembly are described to enable the rapid application of multiple external clasps to a dural graft pre-implanted with multiple couplers.

Additionally, the device may utilize the fixed perpendicular orientation of the graft-clasp unit on the graft-clasp positioning cylinder or an adjustable coupling device to enable rotation of the graft-clasp unit to facilitate visualization of the fenestration and nearby structures.

Within certain embodiments, tissue repair and seal devices disclosed herein are configured for the repair and sealing of fenestrations in the wall of visceral hollow organs, including but not limited to esophagus, stomach, small and large intestine, rectum, bladder, ureter, uterus and vagina. Such fenestrations occur both spontaneously (e.g. tumor, infection), purposefully (e.g. incision or biopsy of the organ during surgery), or inadvertently (laceration or puncture during surgery). Fenestrations in such hollow organ walls usually require repair to prevent intraperitoneal leakage of enteral contents or urine, or ingress of bacteria through uterus or vagina, which can lead to peritonitis or fistula formation. Rapid and watertight repair and sealing of such organs can be accomplished using the tissue repair and sealing device described herein at the time of the procedure, thus preventing leakage and subsequent infection or the need for re-operation. In any of these settings, the graft may consist of autologous, homologous, heterologous, or synthetic materials, either directly incorporated as an integrated graft-clasp unit, or substituted at the time of surgery using the bioresorbable graft frame and holder apparatus. Additionally, in any of these settings the device may utilize the fixed perpendicular orientation of the graft-clasp unit on the graft-clasp positioning cylinder or an adjustable coupling device to enable rotation of the graft-clasp unit to facilitate visualization of the fenestration and nearby structures.

Within certain embodiments, tissue repair and seal devices disclosed herein are configured for the repair and sealing of punctures, perforations or ostomies in abdominal hollow organs after biopsy or removal of a cylinder or cannula. Examples of the biopsy-related uses include perforations of esophagus, stomach, small or large intestine, or rectum occurring during trans-oral or trans-anal endoscopic biopsies, or perforations of the vagina and uterus, or bladder and ureters during trans-vaginal and trans-urethral endoscopic procedures, respectively. In another related embodiment, the tissue repair and sealing described device herein may be used for the percutaneous repair of an ostomy or needle puncture in a hollow organ wall, after removal of a drainage cylinder. This embodiment uses the flexible repair and sealing device advanced through and endoscope or over a guide wire, and may be used for external percutaneous cylinders, drains, or cannulas removed from the esophagus, stomach, small or large intestine, rectum, or bladder (suprapubic cylinder). The graft component in these applications may include either flat grafts of natural or synthetic material, or conical occluder grafts. As above, the benefit of immediate sealing of the cylinder ostomy is the prevention of leakage of internal fluids into the peritoneum or through the skin via the percutaneous cylinder tract.

Within certain embodiments, tissue repair and seal devices disclosed herein are configured for the repair and sealing of fenestrations of body cavities, including but not limited to peritoneum, pleural cavity, inner ear or joint space. Drainage of the pleural cavity via thoracentesis can be complicated by pneumothorax, caused by ingress of air through the puncture site in the pleura. Similarly, percutaneous or endoscopic punctures of the peritoneum for surgical access or abdominal paracentesis (e.g. for drainage or dialysis) may subsequently leak along through the cutaneous incision. Similarly, surgical procedures of the ear or joints may create fenestrations in the tympanic membrane or synovium, respectively. In these situations, the tissue repair and sealing device described herein, in either the rigid or flexible form with flat or conical graft, can immediately seal the puncture site and prevent subsequent complications. Additionally, the device may utilize the fixed perpendicular orientation of the graft-clasp unit on the graft-clasp positioning cylinder or an adjustable coupling device to enable rotation of the graft-clasp unit to facilitate visualization of the fenestration and nearby structures.

Within certain embodiments, tissue repair and seal devices disclosed herein are configured for the repair and sealing of fenestrations of defects in body fascia, including but not limited to abdominal wall, chest wall or muscle and ligament fascia. Defects in these fascial structures can lead to herniation of underlying tissues or wound breakdown. Repair of body fascia using the device described herein may include direct closure of the fascia in open (non-MIS) procedures using single or multiple graft-clamp components applied to the fascial edges, or in the case of a large defect, the incorporation of a free graft of natural or synthetic material, which is secured circumferentially to the defect edges using multiple graft-clasp units. In addition, the flexible or rigid tissue repair and sealing device may be used to close fenestrations in fascia via endoscope or by percutaneous approach. In any of these settings the device may utilize the fixed perpendicular orientation of the graft-clasp unit on the graft-clasp positioning cylinder or an adjustable coupling device to enable rotation of the graft-clasp unit to facilitate visualization of the fenestration and nearby structures.

### Tissue Repair and Sealing Devices Comprising a Biocompatible/Bioresorbable Material

Graft-clasp subassemblies that are used in conjunction with applicator subassemblies in the tissue repair and sealing devices disclosed herein are used in surgical procedures and remain in a patient after a surgical procedure is completed. For any given surgical application, one or more component of these graft-clasp subassemblies will comprise one or more biocompatible and/or bioresorbable material.

The tissue repair and sealing devices of the present disclosure comprise several biocompatible and/or bioresorbable elements configured to place a graft composed of natural or synthetic material on the inner surface of a tissue fenestration, at which time the graft is secured in place by release of a biodegradable clasp mechanism onto the outer surface of the tissue. After passage of the graft through the tissue, and placement to completely cover the inner edges of the defect, a flexible bioresorbable clasp is deployed on the outer surface of the tissue, thereby securing the graft in place and providing an immediate watertight, pressure-resistant, repair and sealing of the defect.

Thus, the present disclosure provides grafts, clasps, and couplers comprising biocompatible and/or bioresorbable materials. In certain aspects, coupling devices and clasps are composed of flexible bioabsorbable material that can be designed to apply the required tensile strength of the radial struts to secure the graft in place, and to be completely absorbed over a period of time which allows healing of the graft to the tissue. As part of the present disclosure, in vitro and in vivo feasibility studies were performed and demonstrated favorable mechanical properties with graft-clasp subassemblies comprising PLA (polylactic acid) bioabsorbable components that improved device and graft deployment, created a superior watertight seal, and reduced time of surgical repair and sealing compared to contemporary suture closing or inlay/fibrin sealant techniques, and confirmed immediate leak-proof sealing and short-term durability in vivo in a porcine laminectomy model.

Within certain embodiments, graft-clasp subassemblies disclosed herein incorporate a biocompatible, non-ferromagnetic, passivated metal or metal alloy to enhance the shape memory and superelasticity characteristics of those component parts the tissue repair and sealing device. Suitable biocompatible, non-ferromagnetic, passivated metal or metal alloys for use in the tissue repair and sealing devices disclosed herein include, but are not limited to, cobalt-based alloys, pure titanium, titanium-based alloys, platinum-based alloys, molybdenum, tungsten, and tantalum alloys. Suitable passivated metal or metal alloy wires for use in detachable graft and clasp assemblies exhibit desirable shape memory and superelasticity characteristics such as those exhibited by nickel-titanium (Nitinol) and/or niobium-titanium.

Biocompatible, non-ferromagnetic, passivated metal or metal alloy have been described in the art that may be adapted for use in the presently disclosed tissue repair and sealing devices. See, for example, U.S. Pat. No. 8,349,249 (“Wachter”) and U.S. Pat. No. 8,992,761 (“Lin”), which are incorporated by reference herein.

The selection of biodegradable, biocompatible polymers that adhere to medical-grade standards and that are suitable for use in the tissue repair and seal devices disclosed herein include polylactic acid (PLA), which is known for its biocompatibility, predictable bio-absorption kinetics and mechanical properties as well as its suitability for use in medical applications. Handpiece parts may be CNC machined from production equivalent materials. Components are tested in vitro using the pressure chamber and MIS simulator paradigms to demonstrate comparable efficacy in dural sealing to that observed in the 3-D printed prototype.

In some embodiments, the graft material without support is flexible enough to be passed through the tissue defect, but also firm enough to retain its shape during positioning. In other embodiments, a thin bioresorbable form ring is bonded to the outer circumference of the graft, which is flexible enough to deform during passage through the defect, then return to its original shape on the inner surface of the tissue. Attached to the center of the graft is a coupler that joins the graft to the clasp and includes a mechanism for reversibly attaching to a graft-clasp positioning cylinder of a tissue repair and sealing device applicator subassembly.

As used herein, the term “bioresorbable” refers to materials that are broken down and absorbed by the body, and thus do not need to be removed manually. Bioabsorbable materials include (1) metals or their alloys, commonly magnesium-based and iron-based alloys and (2) polymers including biopolymers, and copolymers thereof, such as polylactide (PLA), polyglycolide (PGA), polylactide-co-D, L lactide (PDLLA), polylactide-co-glycolide (PLGA), polylactide-co-caprolactone (PLCL), polycaprolactone (PCL), polydioxanone (PDO), polylactide-co-trimethylene carbonate (PL-TMC) which can be customized to meet mechanical performance parameters, biocompatibility, and resorption rates.

Bioresorbable materials may be processed via traditional manufacturing methods including injection molding, extrusion, compression molding and machining. These polymers may also be used in novel manufacturing methods such as electrospinning, selective laser sintering, and fusion deposition modeling.

Biopolymers are available that exhibit good biocompatibility and produce degradation products that are eliminated from the body by metabolic pathways. PLA-based substrates are non-toxic and permit cells to differentiate to, for example, produce extracellular matrix components.

The mechanical properties of bioresorbable materials as well as the ability to prolong the degradation time makes polylactide (PLA) poly(lactide-co-glycolide) (PLGA,) and poly(L-lactide-co-D, L lactide) (PDLLA) particularly advantageous material options. As with suture anchors the addition of calcium phosphate helps promote bone growth, while absorbing at a slow enough rate to allow proper functionality of the implant. This controlled degradation is highly beneficial for this application as the ingrowth of bone tissue into the interference screw region allows for the native tissue fixation of the implanted tendon to occur resulting in better patient outcomes once the bioresorbable screw is completely degraded.

Poly L-lactide-co-D, L lactide (PDLLA) implants have good tensile strength, excellent mechanical and thermal properties. Since most of these applications do not require the implant to be placed under an elevated mechanical load, bioresorbable materials used for these treatments have focused on enhancing the biological response and ability to promote healthy bone regeneration without causing any adverse side effects upon degradation.

Polydioxanone (PDO) polymers can be fabricated to provide materials having a desired degree of flexibility, good mechanical properties, and a fast to moderate degradation profile ranging from about 6 to about 12 months. Poly dioxanone (PDO) polymers are suitable for use in the manufacture of grafts, clasps, and central couplers according to the present disclosure, which are able to secure regenerating tissue systems in place long enough to allow for full healing after which the grafts and sutures degrade and become resorbed by the body. The degradation profile of the depends on multiple factors such as polymer crystallinity, molecular weight, sterilization method, and in vivo environment.

Biocompatible and bioresorbable materials have been described in the art that may be adapted for use in the presently disclosed tissue repair and sealing devices. See, for example, AZoM, Biomaterials, 2630 (2004) (describing the classifications and physical characteristics of biomaterials for use in medical devices); Evonik, Medical Plastics News (describing applications for bioresorbable materials in medical devices); Gilding, Polymer 20(12):1459 (1979) (describing biodegradable polymers, including polyglycolic acid (PGA) and polylactic acid (PLA) homo- and copolymers for use in medical devices, in particular in surgical devices); Kadam, Medical Plastics News 15:22 (2020) (discussing applications for medical polymers for developing efficient medical device technologies); Middleton, Biomaterials 21(23):2335 (2000) (discussing synthetic biodegradable polymers for use in orthopedic devices); Santos, Tissue Engineering 225 (Ed. Daniel Eberli, 2010) (reviewing bioresorbable polymers for use in tissue engineering); and Sheikh, Materials 8:5744 (2015) (reviewing biodegradable materials for use in bone repair and tissue engineering). Each of these scientific and medical articles is incorporated by reference herein in its entirety.

### Tissue Repair and Sealing Devices Comprising a Drug Eluting Graft

Drug-eluting matrices and grafts for use with the tissue repair and sealing devices disclosed herein may include one or more drugs or therapeutic agents including, for example, anti-infectives, antineoplastics, biologicals, cardiovascular agents, central nervous system agents, coagulation modifiers, gastrointestinal agents, genitourinary tract agents, hormones, immunologic agents, and metabolic agents as are well known and readily available in the art. (See, Table 3).

TABLE 3
Exemplary Drug-Eluting Patches for Use with Tissue Repair and Sealing
Devices for the Treatment of Exemplary Diseases and Conditions

| Application Site | Cavity | Target | Representative Therapeutics |
|---|---|---|---|
| Cranial | Cranial | Cerebrospinal | Neurodegenerative Diseases: Neuroprotective agents, |
| | Intradural | Fluid (CSF) | enzyme replacement therapy, biologics |
| | | | Chronic Pain: Long-acting analgesics (morphine, |
| | | | ziconotide, baclofen) |
| | | | CNS Infections: Antifungal, antiviral, or antibiotic |
| | | | therapy |
| | | | Epilepsy: Anti-epileptic drugs |
| | | | Meningeal Tumor (Meningeal carcinomatosis; Dural- |
| | | | based tumors): Localized chemotherapy delivery |
| | Abscess/tumor | Brain | Intraparanchymal Brain Tumors: (Primary or metastatic) |
| | cavity | Parenchyma | Localized chemotherapy delivery (carmustine, |
| | | | temozolomide, bevacizumab) |
| | | | CNS Infections: Antifungal, antiviral, or antibiotic |
| | | | therapy |
| Spine | Spine | CSF | Chronic or Acute Pain: Long-acting analgesics |
| | (Thecal Sac) | | (morphine, ziconotide, baclofen) |
| | | | CNS Infections: Antifungal, antiviral, or antibiotic |
| | | | therapy |
| | | | Spasticity; |
| | | | Tumor (Chemo, Biologics) |
| | Spinal Cord | Spinal cord | Autoimmune (Multiple Sclerosis/Guillian-Barre): |
| | Surface | | Biologic agents |
| | | | Spinal Cord Injury: Neuronal Growth Factor, Gliosis |
| | | | Inhibitor |
| | | | Infection: (Viral myelitis): Anti-viral |
| Vascular | Artery or Vein | Bloodstream | Intravascular Drug Infusion (all categories of IV drugs) |
| | | Systemic | |
| | Artery feeding | Tumor | Chemotherapy with localized distribution |
| | tumor | | |
| Gastrointestinal | Stomach, gall | Intestinal Wall | Autoimmune/Inflammatory (Crohn's, Ulcerative Colitis, |
| | bladder, | | Celiac Sprue; Pancreatitis; Peptic Ulcer Disease, |
| | intestine, | | Cholecystitis): Biologics, Anti-inflammatory |
| | rectum; liver | | Infection: (Diverticulitis, Hepatitis) - Antibiotics, Anti- |
| | | | viral |
| | | | Motility Disorders (Constipation, Diarrhea) |
| | | | Degenerative (Liver disease, Pancreatic disease): |
| | | | Biologics |
| Genito Urinary | Genito Urinary | Ureter/ | Chronic UTI; Tumor |
| | | bladder | |
| Cranial Sinus | Cranial Sinus | Sinus contents | Infection; Tumor |
| Otologic | Otologic | Middle ear; | Infection: (Otitis, Mastoiditis; Labyrinthitis) - |
| | | IAC | Antibiotics, Anti-viral, Anti-vertigo |
| | | | Tumor: Anti-neoplastic |
| Respiratory | Respiratory | Bronchus | Airway Disorders (Asthma; COPD): Bronchodilators, |
| | | | Anti-inflammatory |
| Orthopedic | Orthopedic | Joint Space | Arthritis: Anti-inflammatory, Growth factors, stem cells |
| Ophthalmologic | Globe | Intra-ocular | Degenerative (AMD): Biologics, , AREDS2 |
| | | contents | Supplements, Complement C3 inhibitor, Anti-VEGF |
| | | | Glaucoma: prostaglandin analogs, beta-blockers, alpha- |
| | | | adrenergic agonists, and carbonic anhydrase inhibitor |
| | | | Inflammatory (Uveitis) |

The dural substitute graft on a graft-clasp subassembly is replaced with a drug-eluting wafer composed of one of several biocompatible drug-delivery polymers (e.g., PGLA). The wafer is placed through the dura under direct vision with a burr hole, MIS incision, or via a large-bore spinal needle so that the drug-eluting wafer releases drug into the CSF in known amounts and time periods according to the parameters of the polymer. This method may be applicable for any cavity in the body. In addition, the wafer can be reversed to be placed on the outside of the dura, thus releasing drug (e.g., corticosteroid or opioid) into the epidural space.

Within certain embodiments, the present disclosure provides tissue repair and seal devices for minimally invasive deployment of drug-eluting polymer wafers to achieve continuous cerebrospinal fluid drug delivery and treatment of diseases and conditions of the Central Nervous System (CNS). These devices are configured to deliver a graft through a cylinder as small as 3 mm or, alternatively, in a spinal needle (lumbar puncture). In particular, such devices utilize self-expanding, biodegradable, pre-loaded drug-eluting polymer wafer that is deployed via a tissue repair and seal device that is amenable to minimally invasive placement and retrieval directly into the CSF compartment. In certain aspects of these embodiments, drugs are delivered through (1) a cranial twist drill or burr hole, (2) an MIS approach (e.g., using a 6-10 mm tubular portal), or (3) a large-bore needle lumbar puncture.

### Tissue Repair and Sealing Devices for Lumbar Puncture or Endoscopic Surgery

Within certain embodiments, the present disclosure provides tissue repair and sealing devices for use in endoscopic surgical applications. Components of modified graft-clasp for spine endoscope. The graft is a sponge-like consistency which compresses into a cylinder inside 3 mm applicator cylinder. The external clasp is a disc that slides along the outer surface of the 3 mm cylinder and is secured by passage of barbed suture through the external clasp, which creates a resistance to movement of the clasp.

Sponge graft is compressed into a cylinder comprised of a 3 mm applicator cylinder. An external clasp disc slides distally along the outer surface of the 3 mm cylinder. The cylinder is placed into the small durotomy. Compressed graft passes through durotomy and self-expand to full size, then is pulled up against the inner surface of the dura. The Push Rod advances the external clasp disc distally toward the dura. The external clasp disc slides against the outer surface of the dura and locks in place at the dural surface with barbed suture, stabilizing the graft in place. The graft-clasp subassembly is detached from the applicator subassembly by deploying the graft-clasp release cylinder.

The present disclosure provides tissue repair and sealing devices for use in endoscopic surgical applications. Components of modified graft-clasp for spine endoscope. The graft is a sponge-like consistency which compresses into a cylinder inside 3 mm graft-clasp lock cylinder. The external clasp is a disc that slides along the outer surface of the 3 mm cylinder and locks into a groove at the base of the coupler, after which the coupler cylinder detaches.

Sponge graft is compressed into a cylinder inside a 3 mm graft-clasp lock cylinder. An external clasp disc slides distally along the outer surface of the 3 mm cylinder. The cylinder is placed into the small durotomy. Compressed graft passes through durotomy and self-expand to full size, then is pulled up against the inner surface of the dura. The Push Rod advances the external clasp disc distally toward the dura. The External Clasp disc slides against the outer surface of the dura and locks in place at the coupler locking groove, stabilizing the graft in place. Coupler cylinder is detached and removed with graft-clasp positioning cylinder.

Within other embodiments, the present disclosure provides tissue repair and sealing devices for use in repairing lumbar puncture. A large bore spinal needle (3 mm ID) may be inserted percutaneously in the lower back through dura into the CSF space in the thecal sac, usually to insert a small cylinder for either draining CSF (to lower the pressure) or for instilling drugs. After the procedure, CSF leakage through the puncture hole is common and often causes debilitating headaches; an indwelling drain is cumbersome and has frequent complications and requires hospitalization for insertion and continuous drainage.

FIGS. 33-38 illustrate various aspects of tissue repair and sealing devices for use in endoscopic surgical procedures (e.g., lumbar punctures and gastrostomies) to occlude a large-bore needle puncture or percutaneous ostomy site.

FIGS. 33A-33G show a tissue repair and sealing device used to repair a puncture site with the use of a guidewire. In FIG. 33A is shown a tissue repair and sealing device comprising an applicator subassembly having a graft-clasp positioning cylinder, a graft-clasp lock cylinder, and a detachable graft-clasp subassembly, a deployable clasp, and a coupler, wherein the graft is a conical occluder graft, wherein the graft-clasp positioning and lock cylinders are fabricated out of a flexible material, and wherein the clasp, central coupler, and graft are configured with a central channel to accommodate a guidewire. In certain aspects, the conical occluder graft is comprised of a bioabsorbable material.

In FIG. 33B is shown the deployment of tissue repair and sealing device according to the embodiment presented in FIG. 33A, wherein a conical occluder graft is positioned on an inner tissue surface and the radial struts or spokes of a deployable clasp are positioned on an outer tissue surface to apply pressure against the outer tissue surface, secure the conical occlude graft, and, thereby, repair a tissue fenestration (i.e., a puncture or ostomy site) and create a pressure-resistant, watertight seal.

As shown in FIG. 33C, a guidewire is passed through a large-bore needle that is inserted through a tissue barrier for drainage of fluid. In an alternative aspect of this method, the guidewire may be passed through an indwelling catheter prior to its removal. After removal of the large-bore needle or indwelling catheter, the guidewire remains in place (FIG. 33D). FIG. 33E illustrates the passage of the distal (external) end of the guidewire through the central channel within the conical occluder graft, central coupler, and graft-clasp positioning cylinder. The tissue repair and sealing device is advanced along the guidewire to the puncture site and the conical occluder graft is passed through the puncture hole and positioned against the inner surface of the punctured tissue and the tissue repair and sealing device is deployed by moving the graft-clasp lock cylinder toward the distal end of the graft-clasp positioning cylinder to release the deployable clasp and coupler subassembly (FIG. 33F). The deployable clasp is positioned against and applies pressure to the outer tissue surface to secure the conical occlude graft, repair the puncture, and create a pressure-resistant, watertight seal. The applicator assembly is detached from the detachable graft and clasp assembly, which remains at the puncture site, and the applicator assembly is removed by sliding along the guidewire after which the guidewire is then removed (FIG. 33G).

FIGS. 34A-34F show steps in the deployment of a graft-clasp subassembly 302 for use with a narrow delivery cylinder in an endoscope or large-bore spinal needle comprising a disc-shaped locking clasp 309 and compressible sponge graft 305 according to various tissue repair and seal device embodiments. FIG. 34A shows an external disc clasp which slides over the outer surface of a 1-3 mm delivery cylinder 317. Barbed suture 307 embedded in the graft is brought through two small holes 311 in the external disc clasp which resist the movement of the clasp relative to the suture. FIG. 34B shows sponge-like graft with suture 305 compressed inside the delivery cylinder 317. In FIG. 34C, the delivery cylinder is positioned at the opening of a fenestration 306 in the dura 308 and a central shaft 313 is moved distally to advance the compressed graft. In FIG. 34D the graft 305 has passed through the dural opening and has expanded in the CSF below the inner surface of the dura. The sliding external disc clasp 309 is advanced distally (down arrows) along the outer surface of the delivery cylinder 317 with a cylinder 315 (FIG. 34D) while the barbed suture is retracted proximally (up arrows) holding the graft against the inner dura such that the external disc moves against the outer surface of the dura and the graft-clasp subassembly is held securely by the resistance of the suture (FIG. 34E), after which the applicator subassembly is detached and removed (FIG. 34F) and the excess suture trimmed. (See, Table 4).

TABLE 4
Graft-Clasp Subassemblies for Tissue Repair and Sealing
Devices for Endoscopy and Lumbar Puncture Elements

| | |
|---|---|
| ELEMENT NO. | ELEMENT NAME |

SPONGE GRAFT-DEPLOYABLE CLASP SUBASSEMBLY

| | |
|---|---|
| 302 | Sponge Graft-Clasp Subassembly |
| 307 | Sponge Graft-Clasp Coupler (Ball) |
| 311 | Sponge Graft-Clasp Coupler (Post) |
| 313 | Sponge Graft-Clasp Coupler (Positioning Cylinder) |
| 305 | Sponge Graft-Clasp Graft (Sponge) |
| 309 | Sponge Graft-Clasp Clasp (Locking) |

APPLICATOR SUBASSEMBLY

| | |
|---|---|
| 315 | Applicator Graft-clasp lock Cylinder |
| 317 | Applicator Sponge Graft Positioning Cylinder |

FIG. 35A-E show an alternative configuration of the graft-clasp subassembly 303 for use with an endoscope comprised of internal and external compressible sponges according to various tissue repair and seal device embodiments. FIG. 35A shows the fully deployed graft-clasp subassembly with sponge-like graft or other materials comprising both the internal graft 305 and external clasp 310 connected by a short filament 312. In FIG. 35B, both elements of the graft-clasp subassembly are compressed in a 1-3 mm delivery cylinder which is positioned at the dural fenestration (FIG. 35C), advanced by a push rod 317 and released sequentially (FIG. 35D) to initially position the graft on the inner surface of the dura, followed by the clasp component to secure the position of the graft on the outer surface (FIG. 35E). (See, Table 5).

TABLE 5
Tissue Repair and Sealing Device Graft-Clasp Subassemblies
for Endoscopy and Lumbar Puncture Elements

| | |
|---|---|
| ELEMENT NO. | ELEMENT NAME |

SPONGE GRAFT-SPONGE CLASP SUBASSEMBLY

| | |
|---|---|
| 303 | Sponge Graft-Clasp Subassembly |
| 312 | Sponge Graft-Clasp Coupler (Rod) |
| 305 | Sponge Graft-Clasp Graft (Sponge) |
| 310 | Sponge Graft-Clasp Clasp (Sponge) |

APPLICATOR SUBASSEMBLY

| | |
|---|---|
| 317 | Applicator Sponge Graft Positioning Cylinder |

Devices according to these embodiments can be used to plug a needle puncture hole in the dura after a drain or to insert a drug-eluting graft for drug delivery into the CSF (opioids, anti-spasticity, antibiotics, etc). This figure from the original patent shows a flexible Device applicator passing over a guide wire placed when the drain is removed.

Tunable, zero-order drug release can be achieved over 1-6 weeks, using FDA-approved, biocompatible polymers with known drug delivery characteristics such as polylactic acid (PLA), polyglycolic acid (PLGA) or Polycaprolactone (PCL). Local therapy is achieved with minimized systemic toxicity, improved efficacy, the option of device removal/replacement, and the capability of delivering drugs directly into the CNS.

Polymeric drug delivery offers several advantages pertinent to the treatment of CNS disorders which apply to the presently-disclosed drug delivery device. These include (1) tunable zero- or first-order drug release; (2) biocompatibility and/or biodegradability; (3) localized therapy; and (4) conformability. Lumbar puncture to remove CSF or to administer drugs is done with a large bore needle. Often CSF leaks through the puncture hole. A modified flexible tissue repair and seal device is configured with a central channel and inserted over a guidewire and a bioresorbable plug is placed beneath to hold in the dura and secured with external clasps.

FIG. 36 shows the use of a tissue repair and sealing device comprising an intradural drug-eluting wafer 601 for localized release and administration of a drug to the cerebrospinal fluid (CSF). The wafer is placed inside the dura with the applicator through a large-bore spinal needle and secured with an external clasp (FIG. 36A coronal view; FIG. 36B sagittal view). Drug is released from the polymer in a continuous regulated manner, where it diffuses throughout the CSF (FIG. 36C coronal view; FIG. 36D sagittal view).

FIGS. 37A-37D show the use of a tissue repair and sealing device comprising an extradural drug-eluting wafer for localized release and administration of a drug to an epidural space according to embodiments of the tissue repair and sealing devices disclosed herein. The wafer is placed immediately outside the dura with the applicator through a large-bore spinal needle (FIG. 37A coronal view; FIG. 37B sagittal view. Drug is released from the polymer in a continuous regulated manner, where it diffuses into the epidural space to provide a local effect on the nerves and spine (e.g., opioid, corticosteroid) at the level of administration (FIG. 37C coronal view; FIG. 37D sagittal view).

Polymer-based implants can achieve supratherapeutic drug concentrations in the CNS, with minimal systemic exposure. A variety of physical constructs have been proposed to demonstrate the potential of sustained, local release to maximize CNS exposure, including polyanhydride wafers, PLGA/PLA/PCL co-polymeric discs, nano-particles, microspheres and membranes, lipid-polymer hybrids and PEGylated nanoparticles. Several classes of suitable drug delivery polymers for drug delivery to the CNS are presented in Table 6 and include the FDA-approved Gliadel® wafer (polyanhydride/BCNU) platform for local brain tumor therapy.

TABLE 6
Polymer Drug Delivery Platforms for the Central Nervous System (CNS) *

| | FDA-Approved | | | |
|---|---|---|---|---|
| Polymer Class | Use in CNS | Experimental Use in CNS | Example Drugs | Advantages |
| Polyanhydrides: | Carmustine | Polysebacaic acid (PSA) | NGF, BDNF, | Documented |
| | (Gliadel) | | Anti-inflammatory | efficacy; provide |
| | | PSA-CPP Hybrids - | Temozolomide, | 3-7 day controlled |
| | | | paclitaxel, | release |
| | | | dexamethasone, | |
| | | | lidocaine | |
| | | Other Hybrids | Anti-epileptic, | |
| | | (microspheres or | neuroprotective | |
| | | nanofibers-) | | |
| Polylactic Acid | No CNS Use | PCL-PLGA microspheres | Opioids | Fine-tuned |
| (PLA) | Neurologic Non- | PCL nanoparticles | Neuroprotective | degradation profiles |
| Polyglycolic | CNS Use - nerve | | agents | (days to months |
| Acid (PLGA) | repair scaffolds | PCL/PLGA implants | Anti-inflammatory | |
| Polycaprolactone | | | (spinal cord injury) | |
| (PCL) | | PCL/PLGA Wafers | Glioma cavity | |
| | | Thermoresponsive PLGA- | Paclitaxel, NGF, | |
| | | PEG-PLGA Hydrogels | dexmedetomidine | |
| PEGylation and | Doxorubicin | PEGylated liposomes with | Chemotherapy | BBB Permeability, |
| copolymer blend | PEGylated | surface ligands to cross the | | improved release |
| liposomes | liposomes (not | BBB. | | profiles, |
| | approved for CNS) | PEGylated Enzymes | Mucopolysaccharidoses | programmable |
| | | | (MPS) | mechanical |
| | | Neurotrophic factors | Neurodegenerative | properties |
| | | (BDMNF, NGF) | disorders | |

Where potent therapeutics are none-the-less ineffective when administered systemically because the blood-brain barrier (BBB) and/or blood-CSF barrier sharply restricts drug access to the CNS and clinicians are forced to contend with systemic toxicity from high oral or intravenous doses, unpredictable CNS drug levels due to rapid clearance or low penetration, and lack of durable, local therapy for conditions such as glioblastoma, leptomeningeal metastases, CNS infections, and inflammatory syndromes. Moreover, current localized drug delivery approaches for the CNS require either open surgery (e.g., Gliadel® wafer for carmustine), which are only feasible for select tumor settings, or cannot provide sustained or replaceable therapy to the CSF compartment.

There is no approved minimally invasive method for delivering and retrieving a biodegradable, drug-eluting wafer into the CSF via a lumbar or cranial MIS port, or for combining watertight dural repair with chronic local CNS therapy. Addressing this unmet need could transform the management of multiple CNS disorders by enabling continuous, localized treatment tailored to the patient and disease biology.

Tissue repair and sealing devices according to these embodiments permit the effective treatment of many central nervous system (CNS) conditions including brain and spinal tumors, infections, chronic pain, and neurodegenerative diseases by providing sustained therapeutic drug concentrations within the brain or spinal cord (Table 7) thereby overcoming limitations in the art by (1) substituting a drug-eluting polymer wafer for the graft on the device for delivering drugs to the CSF (opioids, anti-spasmodic agents, anti-neoplastic agents) and to the epidural space just outside the dura (corticosteroids, opioids), (2) configuring the tissue repair and sealing device for delivery through a 3 mm cylinder that permits the placement of a polymer wafer with a lumbar puncture through a large-bore spinal needle, and (3) providing a retrieval mechanism to permit the removal of a drug polymer that causes a reaction or when a polymer has released all of the drug. Wafers delivered through a 3 mm cylinder can simply be pulled out without damaging the dura. Larger wafers may utilize nitinol tines and an umbrella-type mechanism to draw the wafer back into the delivery cylinder.

TABLE 7
Diseases Treatable by Deploying a Tissue Repair
and Sealing Device for Targeted CSF Drug Delivery

| Disorder | Specific Disease | Prevalence* |
|---|---|---|
| Neurodegenerative | Parkinson's Disease | 1,000,000 |
| Disorders | Alzheimer's Dementia | 6,700,000 |
| | Multiple Sclerosis | 1,000,000 |
| | ALS | 30,000 |
| | Huntington's Disease | 41,000 |
| CNS Infections | Bacterial Meningitis | 5,000 |
| | Viral Meningitis | 50,000 |
| | Fungal Meningitis | 2,000 |
| Pain and Spasticity | Chronic Pain | 10,000,000 |
| | Spasticity with Anti-spasticity Medication | 750,000 |
| Brain Tumors | Primary Brain Tumors | 25,000 |
| | Brain Metastases | 250,000 |
| Epilepsy | | 3,400,000 |

*Approximate Number of Cases Annually

Within certain embodiments, tissue repair and seal devices disclosed herein are configured for the continuous, localized delivery of drugs and other agents from a drug-eluting matrix incorporated into, or replacing the graft component. Localized delivery of drugs provides several benefits; (a) the concentration of the drug is highest at the site of application, and untoward effects from systemic distribution of the drug are minimized; (b) the drug can be administered in adequate concentration to body compartments that are relatively inaccessible to the drug administered by intravenous or oral route (e.g. cerebrospinal fluid due to blood-brain barrier restriction, poorly perfused compartments such as abscess cavity); (c) continuous delivery ensures a therapeutic steady-state concentration of the drug without the peak and trough fluctuations which occur with intermittent administration; (d) patient compliance is not an issue; and (e) the drug-eluting matrix can be biodegradable and engineered to release a specific drug at a known rate and duration depending on the site of delivery. Many drug-eluting matrices are currently in clinical use, although most involve implantation of the matrix into subcutaneous or solid tissues. In the current embodiment, any category of drug or bioactive agent could be implanted and secured at any site in the body using the modified tissue repair and sealing device, depending upon the clinical setting and intended therapeutic effect.

The distribution of the drug would depend upon the site of application of the matrix. For example, a matrix placed on the inner surface of a blood vessel could provide systemic distribution for a venous implant site, or provide regional drug distribution to the downstream tissues perfused by an artery (e.g. a neoplasm or single organ). Additionally, a matrix placed on the inner surface of a tissue barrier could provide drug delivery to the fluid or cavity enclosed by the barrier (e.g. dural implant releasing drug into cerebrospinal fluid, peritoneal implant releasing drug into peritoneal cavity, or gastrointestinal implant releasing drug into the bowel). Also, the matrix placed on the inside of a barrier could release drugs to modulate the barrier itself (e.g. promoting healing, inhibiting inflammation, scarring, or hyperplasia, or local pain control). Finally, a matrix implant placed inside the capsule of a solid organ or tumor, in contact with the parenchyma, could provide local drug delivery to that part of the organ or tumor (e.g. kidney, pituitary, malignant or benign tumor). As above, this application of the repair and sealing device could be applied to nearly every category of drugs and every body organ and tissue type.

Suitable drug-eluting polymers for wafer design and preparation include, without limitation, PLA (Polylactic Acid), PLGA (Poly(lactic-co-glycolic acid, POC (copolymers of (poly(diol-citrate)) (POC) and PCL (Polycaprolactone) as unitary polymers or as co-polymers and which are FDA-approved for implantable medical devices, degrade by hydrolysis into lactic/glycolic acid (metabolized safely), and allow precise control of mechanical expansion (as required for delivery through narrow cylinders) and drug release rates.

PEGylated copolymers and blends may be employed to achieve enhanced flexibility and diffusivity, allowing controlled swelling, and to provide mechanical properties that are compatible with deployment, sealing, and tissue apposition. Formulations of the polymer will be considered on the basis of (1) compression for enclosure through introducer cylinders. to <6 mm (lumbar) and <10 mm (cranial); (2) expansion in CSF to 5-20 mm to conform to fit within CSF subarachnoid compartment); (3) mechanical strength to accommodate both secure dural contact and sealing, as well as flexibility for atraumatic deployment and retrieval; (4) drug load capacity to provide clinically meaningful dosing.

PLA, PLGA, and copolymer wafers are fabricated with drug loading for CNS therapeutics and characterized for in vitro drug release and mechanical performance. Using solvent casting and compression molding for PLA/PLGA with variable lactic:glycolic ratios to modulate degradation rates. Integrate drugs (BCNU, dex, peptide) during casting, ensuring even distribution. Test geometries for deployment through 3-6 mm (lumbar) and 6-10 mm (cranial) introducers.

Drug release is assessed in vitro by incubation of the wafer in a chamber replication the fluid dynamics of human CSF. Thus, within certain embodiments, the tissue repair and sealing devices described herein incorporate a drug-eluting matrix to provide a continuous release of drugs to tissues at the site of tissue repair and sealing that comprises a bioresorbable drug-eluting matrix to provide continuous drug delivery to the fluid, tissue or space within a body cavity, blood vessel, lumen or other structures within the body. By placing the drug-eluting matrix on the inner tissue surface, drugs or agents of many types can be locally and continuously released into blood, body fluids, or tissue parenchyma in contact with the matrix. The device can be either rigid or flexible as above, and passed under direct vision, by endoscope, or by a percutaneous approach using a guide wire.

The tissue repair and sealing devices may be adapted for use in securing a drug-eluting matrix, or a graft that incorporates a drug-eluting matrix, to an inner tissue surface including, without limitation, a tissue selected from dura, blood vessel, wall of esophagus, stomach or intestine, bladder wall, ureter, peritoneum, pleura, uterus, Fallopian cylinder, sclera of the eye, synovium, tympanic membrane or the capsule of a solid organ. Drugs that are incorporated into a drug-eluting matrix are released into the fluid or space contained by the tissue barrier (blood, cerebrospinal fluid, gastrointestinal contents, pleural cavity, peritoneal cavity, vitreous humor, inner ear, Fallopian cylinder or joint space) and can be fashioned to disperse drugs at a pre-determined rate and concentration based upon the nature of the drug, the target tissue, and the chemical composition of the drug-eluting matrix to achieve the intended therapeutic effect.

In certain embodiments, tissue repair and sealing devices as disclosed herein may be advantageously employed to provide the continuous delivery of therapeutic agents to the bloodstream via arteries or veins for systemic distribution, to the bloodstream of arteries serving tissues distal to the implant to produce a localized effect in those downstream tissues while minimizing systemic distribution, or to fluids and/or tissues within a cavity or space. In certain applications, the drug that is released can act locally and directly upon the tissue to which the drug-eluting matrix, or graft comprising a drug-eluting matrix, is secured. Thus, the present disclosure contemplates the use of the tissue repair and sealing devices disclosed herein for use in providing the local delivery of agents to promote healing, to prevent local cellular proliferation (e.g., intimal proliferation or excess scar formation), to provide local anesthesia, to inhibit fertilization, or to treat infection with antimicrobial agents. For either embodiment, the bioresorbable nature of the repair and sealing device and drug matrix would eliminate the need for removal of the device at the conclusion of therapy.

Drug-eluting matrices and grafts have been described in the art that may be adapted for use in the presently disclosed tissue repair and sealing devices. See, for example, Alvarez-Lorenzo, Journal of Pharmacology and Experimental Therapeutics 370:544 (2019) (describing implantable smart drug release devices and materials); Concheiro, Advanced Drug Delivery Review 65(9):1188 (2013) (describing chemically cross-linked and grafted cyclodextrin hydrogels for use in drug-eluting medical devices); Nie, Journal of Materials Chemistry 7:6515 (2019) (describing integrated grafts comprising a biologically developed cartilage-bone interface of osteochondural defect repair); Zilberman, 299 (Springer-Verlag 2010) (reviewing drug-eluting medical implants, including drug-eluting matrices and grafts); Zilberman, Journal of Controlled Release 130(3):202 (2008) (describing antibiotic-eluting medical devices, including drug-eluting matrices and grafts); Zuckerman, Gels 6:9 (2020) (describing affinity-based release from cyclodextrin hydrogels); Richter, U.S. Pat. No. 7,048,714 (describing drug eluting medical devices having an expandable portion for drug release); Ding, U.S. Pat. No. 7,758,909, Lye, U.S. Patent Publication No. 2005/0070989, and Feng, U.S. Patent Publication No. 2008/0051881 (describing medical devices having a porous surface/layer for controlled drug release); Fennimore, U.S. Pat. No. 8,007,737 (describing antioxidants for the prevention of oxidation and degradation of drugs in drug-eluting medical devices); Atanasoska, U.S. Pat. No. 8,815,273 (describing drug-eluting medical devices having porous layers); Jennings, U.S. Pat. Nos. 9,605,175 and 10,314,912 (describing polymer coating compositions for use in medical devices); Gemborys, U.S. Pat. Nos. 9,801,983 and 10,159,769 (describing medical devices for delivering bioactives to a point of treatment); Speck, U.S. Patent Publication No. 2011/0295200, Zilberman, U.S. Patent Publication No. 2016/0082161, and Hoffmann, U.S. Patent Publication No. 2011/0301697 (describing drug-eluting medical devices); Wong, PCT Patent Publication No. 2006/135609 (describing asymmetric drug-eluting hemodialysis grafts); Hanson, PCT Patent Publication No. 2008/156487 and Peck, PCT Patent Publication No. 2014/144188 (describing drug-eluting grafts for the local drug delivery to tissues). Each of these scientific and medical articles, patents, and patent publications is incorporated by reference herein in its entirety.

Within certain embodiments, the present disclosure provides tissue repair and sealing devices for the rapid dural closure of long incisions in open (non-MIS) surgical procedures. In open (non-MIS) cranial and spine neurosurgical cases, there is often a long incision (10 cm or more) which must be closed. This is done with suturing, which can take 20-30 minutes. Also, the brain is not visible as the dura re-approximates, and there is risk of lacerating the brain with the suture needle. In this application, curved rectangular Device grafts are sequentially placed with the graft under the dura and secured with external clasps in the usual fashion.

Often there is a large defect in the dura from tumor or trauma, which necessitates repair using a free graft (synthetic or tissue). The free graft must be carefully sutured circumferentially, which is time consuming and holds a small risk of injury to underlying brain. In this case, grafts are placed around the free graft and secured to the edges of the dural defect.

Within other embodiments, the present disclosure provides tissue repair and sealing devices for long incisions in open (non-MIS) procedures using a modification of the Device applicator rapidly to multiple couplers embedded in a dural substitute graft. In this repair, the dural substitute graft are fabricated in a long strip with the couplers pre-installed. A modified applicator is like a stapler, holding multiple external clasps that can be sequentially released one at a time. The strip of dural substitute graft is placed under the dural incision with the upper portion of the coupler exposed on the outside. The applicator then placed external clasps sequentially along the graft until clasps are securing the underlying graft at each coupler.

Within other embodiments, the present disclosure provides tissue repair and sealing devices for closing dura at a burr hole. Small holes (burr hole=12-16 mm; twist drill hole=5 mm) are frequently drilled through the skull for a number of reasons; (1) drain hematomas or infections, (2) insert shunt or drainage catheters into the ventricle, (3) insert electrodes for brain stimulation or epilepsy monitoring, (4) insert brain-computer interface, and increasingly (5) for endoscopic tumor or hemorrhage surgery). The dura is incised in each case but is difficult to suture due to lack of space. The standard Device graft can be used to seal the dural incision.

In other embodiments, the present disclosure provides tissue repair and sealing devices that employ a tissue sealant (e.g., Tisseal® fibrin or DuraSeal® polymer) added at the conclusion of a dural repair as an adjunct to sealing. These tissue sealants comprising 2-components that polymerize quickly when mixed and are injected at the dural repair site where they mix and polymerize in situ. The injector can obscure visualization of the repair in MIS, and requires the surgeon to remove dural repair instruments to bring the injector into the field. In this iteration, the two cylinders for sealant components are embedded in the wall of the Device graft-clasp lock cylinder, so that the sealant can be injected over the Device graft at the repair site under direct vision without changing the field of view.

In further embodiments, the present disclosure provides interchangeable graft-clasp cartridges for use in the tissue repair and sealing devices disclosed herein. Interchangeable graft cartridges are 4-6 mm cassettes for use in combination with an applicator subassembly as disclosed herein. Because different graft sizes require different graft-clasp lock cylinder sizes, a complete new device would be required if the surgeon finds he/she needs a different size or wants to use a different graft material. The new handpiece design allows an interchangeable cassette consisting of the desired size/material folded graft enclosed in an appropriate-sized cylinder, with the matching size external clasp on the cylinder. The cassette would attach to the distal end of the universal handpiece immediately before use. A selection of cassettes would be sterilized in separate packaging and available in the OR to meet the surgeon's needs.

For tissue repair and sealing devices engineered for repair of small durotomy (2-3 mm) through a spine endoscope working channel (Inside Diameter 7 mm), the device is modified to accept an interchangeable cassette at the end of the graft-clasp lock cylinder. The cassette contains the folded graft and the external clasp that are appropriately sized to the durotomy, and is fastened to the distal end of the graft-clasp lock cylinder. In this manner, the surgeon can choose a graft ranging from 4-8 mm in diameter (for a durotomy 2-6 mm), which is attached to the applicator handpiece during the procedure to best accommodate the durotomy size.

Within certain embodiments, the present disclosure provides tissue repair and sealing devices for use in endoscopic surgical applications. (A) Components of modified graft-clasp for spine endoscope. The graft is a sponge-like consistency which compresses into a cylinder inside 3 mm applicator cylinder. The external clasp is a disc that slides along the outer surface of the 3 mm cylinder and is secured at the dural surface by barbed sutures attached to the graft, which draw and hold the disc against the outer dural surface. (B) Sponge graft is compressed into a cylinder inside a 3 mm graft-clasp lock cylinder. An external clasp disc slides distally along the outer surface of the 3 mm cylinder. The cylinder is placed into the small durotomy. (C) Compressed graft passes through durotomy and self-expand to full size, then is pulled up against the inner surface of the dura. The Push Rod advances the external clasp disc distally toward the dura. (D) The External Clasp disc slides against the outer surface of the dura and is secured at the dural surface by barbed sutures attached to the graft, stabilizing the graft in place. (E) Sutures are trimmed with graft-clasp positioning cylinder and graft-clasp lock cylinder are removed.

Within other embodiments, the present disclosure provides tissue repair and sealing devices for use in repairing lumbar puncture. A large bore spinal needle (3 mm ID) may be inserted percutaneously in the lower back through dura into the CSF space in the thecal sac, usually to insert a small cylinder for either draining CSF (to lower the pressure) or for instilling drugs. After the procedure, CSF leakage through the puncture hole is common and often causes debilitating headaches; an indwelling drain is cumbersome and has frequent complications and requires hospitalization for insertion and continuous drainage.

Devices according to these embodiments can be used to plug a needle puncture hole in the dura after a drain or to insert a drug-eluting graft for drug delivery into the CSF (opioids, anti-spasticity, antibiotics, etc). FIGS. 33A-G shows a flexible Device applicator passing over a guide wire placed when the drain is removed.

### EXEMPLARY EMBODIMENTS

While various embodiments have been disclosed herein, other embodiments will be apparent to those skilled in the art. The various embodiments disclosed herein are for purposes of illustration and are not intended to be limiting, with the true scope and spirit being indicated by the claims. The present disclosure is further described with reference to the following examples, which are provided to illustrate certain embodiments and are not intended to limit the scope of the present disclosure or the subject matter claimed.

1. A tissue repair and sealing device for use in an open (non-MIS) or minimally invasive surgical (MIS) procedure for rapidly repairing a tissue fenestration and creating a pressure-resistant, watertight seal, said device comprising: a. an applicator assembly comprising an graft-clasp positioning cylinder having a proximal end and a distal end and a graft-clasp lock cylinder having a proximal end and a distal end, wherein said graft-clasp lock cylinder is movably connected to said graft-clasp positioning cylinder and b. a detachable graft and clasp assembly comprising, in operable combination, a graft subassembly comprising a graft fixedly attached to a deployable clasp and coupler subassembly comprising a deployable clasp and central coupler.

2. A tissue repair and sealing device for use in an open or minimally invasive (MIS) surgical procedure to rapidly repair a tissue fenestration and create a pressure-resistant, watertight seal, said device comprising (a) an applicator subassembly comprising, in operable connection, (i) a deployable graft-clasp lock mechanism, (ii) a deployable graft-clasp release mechanism, and (iii) a deployable graft-clasp positioning mechanism and (b) a graft-clasp subassembly comprising, in operable connection, (i) a foldable graft, (ii) a deployable clasp, and (iii) a bioabsorbable coupler.

3. a tissue repair and sealing device of embodiment 1 or 2 wherein said graft-clasp lock mechanism comprises a first cylinder having a first diameter, wherein said graft-clasp release mechanism comprises a second cylinder having a second diameter that is less than said first diameter, wherein said graft-clasp positioning mechanism comprises a third cylinder having a third diameter that is less than said second diameter, wherein said first, second, and third cylinders are aligned concentrically (i.e. nested), and wherein each of said first, second, and third cylinders has a proximal end and a distal end.

4. The tissue repair and sealing device of embodiments 1-3 wherein deploying said graft-clasp positioning mechanism permits the positioning of a graft on a tissue inner surface and a clasp on a tissue outer surface.

5. The tissue repair and sealing device of embodiments 1-4 wherein deploying said graft-clasp lock mechanism locks a clasp into a graft-clasp coupler on said graft-clasp subassembly.

6. The tissue repair and sealing device of embodiments 1-5 wherein deploying said graft-clasp release mechanism disconnects the graft-clasp subassembly from the applicator subassembly at the distal end of said graft positioning cylinder.

7. The tissue repair and sealing device of embodiments 1-6 wherein said clasp comprises one or more: (1) expands [based on material characteristics] and (2) is water impermeable.

8. The tissue repair and sealing device of embodiments 1-7 wherein said clasp comprises one or more: (1) curved tips to facilitate strut sliding while locking, (2) folded toward distal end of the applicator subassembly, (3) a central ring with radial struts, and (4) inner flexible posts that pass over a coupler ball to lock onto coupler groove.

9. The tissue repair and sealing device of embodiments 1-8 wherein said coupler comprises one or more: (1) is not integrated with an external clasp and (2) comprises a locking groove for a corresponding clasp strut ring.

10. The tissue repair and sealing device of embodiments 1-9 wherein said applicator comprises one or more: (1) a stationary external constraining cylinder, (2) multiple bushings for graft and clasp to move within an external constraining cylinder, (3) an additional bushing to push ring onto locking groove, (4) a mechanism to disarticulate applicator from coupler, (5) a grip rocker trigger to actuate various bushings, (6) a grip thumb lever to disarticulate a central cylinder from a coupler ball, and (7) a grip cylinder for administration of sealant.

11. The tissue repair and sealing device of embodiments 1-10 wherein said deployable clasp assembly comprises one or more: (1) is configured to adopt a folded configuration when retained by said graft-clasp lock cylinder and to rapidly unfold to a pre-folded state.

12. The tissue repair and sealing device of embodiments 1-11 wherein said deployable clasp comprises a biopolymer selected from the group consisting of a polylactide (PLA), a polyglycolide (PGA), a polylactide-co-D, L lactide (PDLLA), a polylactide-co-glycolide (PLGA), a polylactide-co-caprolactone (PLCL), a polycaprolactone (PCL), a polydioxanone (PDO), and a polylactide-co-trimethylene carbonate (PL-TMC), wherein said biopolymer exhibits shape memory and superelasticity characteristics that permit the folding of said biopolymer while retaining the capacity to rapidly unfold to a pre-folded state.

13. The tissue repair and sealing device of embodiments 1-12 wherein said deployable clasp assembly comprises a biocompatible, non-ferromagnetic, passivated metal or metal alloy wire that is selected from the group consisting of pure titanium; a titanium-based alloy; a cobalt-based alloy; a platinum-based alloy; and a molybdenum, tungsten, and tantalum alloy, wherein said biocompatible, non-ferromagnetic, passivated metal or metal alloy wire exhibits shape memory and superelasticity characteristics that permit the folding of said wire while retaining the capacity to rapidly unfold to a pre-folded state.

14. The tissue repair and sealing device of embodiments 1-13 wherein said biocompatible, non-ferromagnetic, passivated metal or metal alloy is selected from the group consisting of a nickel-titanium alloy (Nitinol) and a niobium-titanium alloy.

15. The tissue repair and sealing device of embodiments 1-14 wherein said graft assembly is configured (a) to adopt a folded configuration when traversing a tissue fenestration or when retained by said graft-clasp lock cylinder and (b) to rapidly unfold to a pre-folded state.

16. The tissue repair and sealing device of embodiments 1-15 wherein said graft is selected from the group consisting of an autograft, an isograft, an allograft, and a xenograft and wherein said graft is derived from an animal tissue is selected from the group consisting a human tissue, a bovine tissue, and a porcine tissue.

17. The tissue repair and sealing device of embodiments 1-16 wherein said graft material comprises one or more synthetic material selected from the group consisting of poly(ethylene terephthalate) and expanded polytetrafluoroethylene (ePTF).

18. The tissue repair and sealing device of embodiments 1-17 wherein said graft comprises an acellular, porous extracellular matrix scaffold of collagen, elastin, and, optionally, a growth factor.

19. The tissue repair and sealing device of embodiments 1-18 wherein said graft comprises a dural substitute selected from the group consisting of Duraform® dural graft implant, Biodesign® Dural Graft, DuraGen® Matrix, DuraMatrix® graft, Cerafix dural Graft®, PRECLUDE®, Lyoplant Onlay Graft®, Neuro-Patch Dural Graft®, SEAMDURA®, and Durepair™ Regeneration Matrix.

20. The tissue repair and sealing device of embodiments 1-19 wherein said graft comprises a drug eluting matrix.

21. The tissue repair and sealing device of embodiments 1-20 wherein said graft comprises a biocompatible, non-ferromagnetic, passivated metal or metal alloy wire that is selected from the group consisting of pure titanium; a titanium-based alloy; a cobalt-based alloy; a platinum-based alloy; and a molybdenum, tungsten, and tantalum alloy, wherein said biocompatible, non-ferromagnetic, passivated metal or metal alloy wire exhibits shape memory and superelasticity characteristics that permit the folding of said wire while retaining the capacity to rapidly unfold to a pre-folded state.

22. The tissue repair and sealing device of embodiments 1-21 wherein said biocompatible, non-ferromagnetic, passivated metal or metal alloy is selected from the group consisting of a nickel-titanium alloy (Nitinol) and a niobium-titanium alloy.

23. The tissue repair and sealing device of embodiments 1-22 wherein said deployable clasp assembly is configured to adopt a folded configuration when retained by said graft-clasp lock cylinder and to rapidly unfold to a pre-folded state.

While various embodiments have been disclosed herein, other embodiments will be apparent to those skilled in the art. The various embodiments disclosed herein are for purposes of illustration and are not intended to be limiting, with the true scope and spirit being indicated by the claims. The present disclosure is further described with reference to the following examples, which are provided to illustrate certain embodiments and are not intended to limit the scope of the present disclosure or the subject matter claimed.

### EXAMPLES

### Example 1

### In Vitro and In Vivo Model Systems for Testing Tissue Repair and Sealing Devices

The Examples that follow provide in vitro and in vivo model systems for assessing the mechanical attributes and performance characteristics of tissue repair and sealing devices under conditions of unconstrained access (no limitation of size of field or angle of approach as during an open (non-MIS) surgical procedure) and under conditions of constrained access (limitation of size of access portal and/or angle of approach as during a minimally invasive (MIS) surgical procedure). Visualization, access, deployment, and sealing efficacy have been confirmed under constrained conditions using (1) a pressurized MIS model using an MIS burst chamber (ASTM F2392) and (2) a pressurized MIS spine laminotomy model with narrow access portals, endoscopic vision, and limited angles of approach to a model of the human lumbar spine. See, Table 8.

TABLE 8
Burst Chamber Design Clinical Simulators for In Vitro Testing

| | Clinical | | |
|---|---|---|---|
| Simulation | Equivalent | Apparatus | Camera |
| Unconstrained | Open | Pressure Chamber | Internal and |
| Access | Procedures | | External to Dura |

| | Clinical | | Angle of Device | Access Port |
|---|---|---|---|---|
| Simulation | Equivalent | Apparatus | Insertion | Diameter |
| Constrained Access | Generic MIS | Endoscopic | 0° | 14/22 mm |
| | Procedures | | 30° | 14/22 mm |
| MIS Spine | MIS Spine | Endoscopic | 30° | 14/22 mm |
| | Simulator | | | |

In addition to the in vitro model systems disclosed herein, the scientific, medical, and patent literature describe in vitro model systems that can be configured for testing the repair and sealing of tissue fenestrations with the devices disclosed herein. See, Dafford, The Spine Journal 15(5):1099 (2015); Chauvet, Acta Neurochirurgica 153(12):2465 (2011); and Wang, MATEC Web of Conferences 119:01044 (2017).

In vitro model systems that use fresh porcine dura for testing acute burst pressures and resistance to intra-cranial pressure and assessing cerebrospinal fluid leakage in repaired and sealed tissue fenestrations are presented in Van Doormaal, Operative Neurosurgery 15(4):425 (2018) and Kinaci, Expert Review of Medical Devices 16(7):549 (2019).

In vitro model systems that use human cadaveric dura mater attached to a cylindrical metal glass filled with colored saline for measuring the water-tightness of repaired and sealed tissue fenestrations and for assessing the pressure at which a repaired and sealed tissue fenestration leaks are presented in Megyesi, Neurosurgery 55(4):950 (2004); Chauvet, Acta Neurochir (Wien) 153(12):2465 (2011); and Kizmazoglu, Br. J. Neurosurgery 33(6):655 (2019).

A porcine dura in vitro model system using a closed testing apparatus utilizing an infused saline solution to provide unidirectional pressure for determining mean failure pressures of repaired and sealed tissue fenestrations is presented in Lin, International Forum of Allergy and Rhinology 6(10):1034 (2016); Lin, International Forum of Allergy and Rhinology 5(7):633 (2015); Chorath, Allergy & Rhinology 10:1 (2019); and Chen, American Journal of Rhinology and Allergy 33(6):757 (2019).

3D printed burst test chambers that emulate different dural repair clinical scenarios were designed with regulated pressure, interchangeable dural membranes, reservoirs for collecting and measuring fluid leak and various access limitations of portal size and angle of approach. Burst chambers included a calibrated digital manometer with computer interface and servo-infusion pump to produce and record accurate internal pressures, and reservoirs to collect and measure fluid leakage. Burst chambers can also include cameras for viewing the dural membrane from above and below to access graft deployment and potential insertion trauma to the dura.

Because of inconsistencies in the thickness and waterproof integrity of commercially-available bovine dura, for both open and MIS sealing efficacy studies bovine pericardium (DuraGuard) was used to provide a more consistent and reliable membrane with structural characteristics nearly identical to human dura and is used in standard models for simulated dural repair.

In practice, a chamber was filled with normal artificial CSF and a baseline pressure of 35 cm H₂O before dural incision to confirm initial watertightness of the chamber. Dura are observed and recorded by cameras above and below the membrane through optical fiber cameras. Leak rate at the site of the dural repair was quantitated by the absorptive paper collection technique (cc/min). At 0 cm H₂O, a standardized 10 mm×10 mm cruciate incision is made in the center of the dural membrane and air bled from the chamber.

Commercially available synthetic graft material (DuraMatrix Basic) precision laser-cut into 16 mm circles are used for the graft, and the graft-clasp with 3-D printed PLA components is assembled and loaded into the applicator. Dural repairs were performed without either access cylinder or angular restriction. Repair sites were not augmented with fibrin sealant. Under servocontrol, artificial CSF was progressively added to the chamber to generate mean pressures ranging from 5-35 cm H₂O in 5 cm H₂O increments at 3 minute intervals (total time=21 min). Outcome measures included leak rate, failure pressure, burst pressure, and procedure completion time.

Pressure chambers were designed to be adjustable to meet the demands of various testing procedures. The body is made of a schedule 80 PVC Tee fitting that has been outfitted with two flanges and an end cap. On the left-side, the end cap is drilled and tapped for a push connect. cylinder fitting that will act as the influent port for our test fluid. This fluid flow is run through a three-way valve with one port controlled by a solenoid valve and the other by a syringe allowing for two different methods of controlling fluid flow.

On the right-side of the tee there is a flange upon which a membrane is fastened by a piece of acrylic. This acrylic has been designed to mount a 6.5″ speaker that will allow for testing of pressure changes created by sound waves that mimic the body's natural respiratory cycle and other human functions. On the top of the tee there is another flange which will hold the test bed. The test bed consists of two pieces of acrylic that will sandwich a piece of commercially-available synthetic Dural material. On the underside of the lower acrylic plate is the pressure transmitter which will monitor the changes in pressure for testing while also controlling the solenoid valve.

Pressure variance via an external speaker that creates waveforms similar to those created naturally by the body, including the natural rhythms of CSF flow, patient movements, coughing and sneezing. The seal is created by the overlap of the dura and the base material. Key physical forces relied on for a watertight seal are the backpressure of CSF, uniform load from the tension arms, and the coefficient of friction between the two surfaces. Back pressure of CSF varies constantly depending on the patient's body and movements. The load applied on the dura varies with the size of each device due to material properties of PLGA. The coefficient of friction helps hold the device in place. A leak that occurs due to any of these forces is overcome in testing.

### Example 2

### In Vitro Model Systems for Testing Tissue Repair and Sealing Devices for Use During an Open (Non-MIS) Surgical Procedure Under Conditions of Unconstrained Access

This Example discloses in vitro model systems for testing the mechanical attributes and performance characteristics of tissue repair and sealing devices under conditions of unconstrained access that approximate the conditions of an open (non-MIS) surgical procedure.

Intact commercially available synthetic dural substitute materials were tested for optimal mechanical deployment characteristics and water permeability using the burst chamber. Video recordings from cameras viewing the dural membrane were taken from above and below and were analyzed to access complete graft deployment (graft area as percentage of non-deployed graft), potential insertion trauma (length as percentage of original dural incision) and accurate deployment and locking of the external clasp during 10 deployments and removals of grafts through the standard 10 mm×10 mm.

FIG. 39 is a graph showing the strut force applied to a surface as a function of the distance (mm) from abase. Loading the external clasp component into the graft-clasp lock cylinder typically deforms the PLA struts to approximately 45 degree angle with approximately 6 mm elevation above the surface. The deformed external clasp component was placed onto a scale, and a precision actuator was used to deflect it downward, and force measurements (grams) were taken at each 0.5 mm increments until the clasp was at its locking position within 0.5 mm of the surface (N=3).

To measure strut force applied to the surface (FIG. 39), struts were placed on a platform scale and displaced downward by a precision actuator (N=3) with strut surface force measured as the displacement load (gm) per 5 mm downward displacement of the coupler, as measured by the load cell. In the locking position, maximum force (dis-placement load) against the surface of the 3 struts combined was 9-13 gm.

Lateral graft stability was assessed (FIG. 40) for a deployed graft in bovine dura mounted in chamber with a 2 cm linear dural incision under internal pressures of 0-30 cm H₂O, defined as the minimum surface force (N) required to prevent lateral displacement. Force to produce lateral movement of the graft in-creased with increasing chamber pressure from 0.2N (0 cm H₂O) to 0.8N (35 cm H₂O).

Device and experimental protocols were consistent with ASTM F2392-04 (surgical sealant protocol). Because of inconsistencies in the waterproof integrity of bovine dura, bovine pericardium (DuraGuard) was used as a dural membrane, which provided a more consistent and reliable membrane with structural characteristics nearly identical to adult human dura. Bovine pericardium has been used in models for simulated dural repair.

The pressure chamber was filled with normal saline, the intact dural membrane secured and a baseline pressure of 35 cm H₂O established to confirm initial watertightness of the chamber and dura. All dural repairs were performed using either Device graft, sutures, or inlay. In the unconstrained model, dural repairs were accomplished without access cylinder or angular restrictions.

FIG. 40 Lateral Force Stability Testing: A wire attached to the coupler base of a Patch-clamp graft deployed in bovine dura mounted in the pressure chamber over pressures from 0-35 cm H₂O was connected to a load cell and force in newtons required to initiate lateral movement was measured (N=5).)

Commercially available synthetic graft material (DuraMatrix Basic) was precision laser-cut into 16 mm circles, and the graft-clasp unit was assembled and loaded into the applicator. Three experimental groups were pressure tested as above following repair with (1) the tissue repair and seal device (N=10); (2) standardized interrupted suture repair with 5-0 Neurolon augmented with onlay synthetic graft (N=10) (DuraMatrix Onlay Plus), or (3) inlay synthetic graft (N=6) (DuraMatrix Onlay Plus) manually placed below the dural incision.

The suture and inlay control repairs were augmented with a coating of fibrin sealant (Tisseel®). Under servo-control, artificial was progressively added to the chamber to generate mean pressures ranging from 5-35 cm H₂O in 5 cm H₂O increments at 3 minute intervals. Fluid leaking through the membrane was collected in a reservoir and absorbed by a paper towel at the end of 3 minutes, and volume (cc) determined by difference in weight (gm) of the towel before and after absorption of fluid.

Outcome measures included (a) Leak Rate (cc/min); (b) Failure Pressure at which the watertight seal on the repair initially failed; and (c) Burst Pressure with complete loss of repair integrity. Failure and burst pressures rates were first compared using a simple log-rank test. In a second analysis, cumulative incidence of failure and burst rates were compared using Cox proportional hazards regression models with multivariable adjustment for: simulated surgery condition (open, MIS-0, MIS-30, spine) and portal size (none, 14 mm, 22 mm).

Tissue repairs that exceeded experimental pressures of 100 cm H₂O without failure or bursting were censored at 100 cm H₂O. A global Chi square test was used to compare failure and burst rates across repair groups in the multivariable Cox regression model. If the global test was statistically significant, a second Chi square test was used to evaluate Device performance against either of the other repair techniques. Reported P values are com-pared against a two-sided type 1 error rate of 0.05 without adjustment for multiple testing. Failure and burst pressures were commonly observed for suture and inlay repairs in the 5-35 cm H2O range. Device repairs performed significantly better than control methods (p>0.001), with a median burst pressure of well over 100 cm H₂O. Failure pressure results showed a similar significant difference (p>0.001) between Device and control groups. Mean failure pressures were: Device: 90 cm H₂O; sutures: 10 cm H₂O; inlays: 20 cm H₂O. Results for cumulative burst rate under open conditions are shown in FIGS. 42-43.

### Example 3

### In Vitro Model Systems for Testing Tissue Repair and Sealing Devices for Use During an MIS Surgical Procedure Under Conditions of Constrained Access

This Example provides in vitro model systems for assessing the mechanical attributes and performance characteristics of tissue repair and sealing devices under conditions of Constrained Access.

The constrained access spine model utilizing a dural incision in bovine dura is presented in FIG. 32A. Measurements from the internal surface of the dura showed: (a) no change in the two incision lengths (95.6% and 100.7%) compared to pre-insertion; (b) rapid (<2 sec) restoration of graft shape and area compared to non-deployed grafts (2.074 cm² vs 2.010 cm²) and (c) 100% sequential extra-dural clasp strut release and locking to the coupler.

A minimally invasive (MIS) surgical procedure (e.g., a human lumbar MIS laminotomy model) with narrow access portals, endoscopic vision, and limited angles of approach. Following fabrication of improved burst test chambers and a lumbar MIS laminotomy simulator.

A pressurized MIS burst chamber constrained access model used MIS access ports having an inside diameter of 14 mm or 22 mm and a length of from 60-90 mm. The MIS access ports are positioned just above dural membrane in an MIS Simulator. In 4 groups of 5 dural repairs each, 14 mm or 22 mm port; 0° or 30° approach angle, a standardized 10 mm cruciate incision was repaired though the access cylinder using the 16 mm circular Dura Matrix Basic graft (not augmented with fibrin sealant). Artificial CSF was progressively added to the chamber to generate mean pressures from 5-35 cm H₂O at 5 cm increments for 3 min each. Leak rate, failure pressure, burst pressure, and procedure completion time were assessed.

Constrained conditions were also obtained with a pressurized spine laminotomy model employing a 3D-printed plastic model of normal human lumbar spine with a typical MIS laminotomy is fitted with a hollow interchangeable cassette in the spinal canal containing a curved watertight dural membrane, situated immediately below the laminotomy. The cassette was pressurized with the servomanometer, tested for water-tightness at 35 cm H₂O and placed in the MIS simulator such that a 10 mm×10 mm cruciate incision in the curved dural exposure was at a 30° angle to the vector of approach.

In 2 groups of 5 experiments each (14 mm vs 22 mm access port), incremental pressure intervals were used to compare leak rate, failure pressure, burst pressure and completion time for grafts with IM vs 3D-printed.

Visualization, access, deployment, and sealing efficacy were assessed under constrained conditions using a pressurized spine laminotomy model (C). A plastic model was 3D-printed from a normal human lumbar spine CT, and the lateral aspect of the left L4 lamina removed in accordance with a typical MIS laminotomy. A hollow interchangeable cassette was printed to fit the spinal canal and contain a curved watertight aperture for the dural membrane, which was situated immediately below the laminotomy. An inlet cylinder to the cassette was connected to the servo-manometer and the cassette tested for water-tightness at 35 cm H₂O.

The laminotomy model containing the pressurized cassette was placed in the MIS simulator such that the curved dural exposure was at a 30° angle to the vector of the access port. After a standardized 1 cm cruciate dural incision, in 4 groups of 5-8 experiments each, an identical protocol as the pressure chamber and MIS experiments with incremental pressure intervals was used to compare leak rate, failure pressure, burst pressure and completion time for grafts vs. inlay graft (Tisseel); as above, suture repair with this model could not be achieved. Repairs performed significantly better (p>0.001), with a median burst pressure of well over 100 cm H₂O while the inlay median burst pressure was 14 cm H₂O. Failure pressure results showed a similar significant difference (p>0.001) between Device and inlays. Mean failure pressures were Device: 100 cm H₂O; inlays 5 cm H₂O. The cumulative failure rate by pressure and repair type across all conditions shows a similarly dramatic difference between Device performance and suture/inlay.

Visualization, access, deployment, and sealing efficacy were evaluated under constrained conditions with endoscopic vision, narrow access portals and various angles of approach using both a pressurized burst test chamber simulates either cranial or spinal MIS dural repair and a pressurized spine laminotomy model. Interchangeable plastic access ports (ID. 14 mm or 22 mm; length 60 mm) were directed through an opening in the MIS simulator to a position just above the dural membrane. In 8 groups of 5-9 dural repairs each, a standardized 10 mm cruciate incision was made with scalpel, and repaired under endoscopic vision though the access cylinder by an experienced neurosurgical fellow using either the Patch-clamp device with a 16 mm DuraMatrix Basic graft or circular DuraMatrix Onlay Plus in-lay graft augmented with Tisseel. For 4 suture repair groups, the neurosurgeon made 2 unsuccessful attempts each lasting 5-10.

FIGS. 42-43 show cumulative burst rate by pressure and repair type stratified under constrained conditions using a pressurized burst chamber for MIS angles 0 (left) and 30 (right). minutes to suture the dural incision with standard MIS needle drivers using 5-0 Neurolon suture at each port size and angle. Mean pressures from 5 cm-35 cm H₂O at 5 cm increments were generated for 3 min each. Outcome measures again included leak rate, failure pressure, burst pressure, and procedure completion time. User experience assessments used a 5-point Likert scale to assess ease of use, advantages, and specific limitations of the device.

Failure and burst pressures were commonly observed for inlay repairs in the 5-35 cm H2O range; Device repairs performed significantly better (p>0.001), with a median burst pressure of well over 100 cm H₂O while the inlay median burst pressure was 25 cm H₂O. Failure pressure results showed a similar significant difference (p>0.001) between Device and inlays. Mean failure pressures were (1) MIS-0: Device: 99 cm H₂O; inlays 15 cm H₂O. (2) MIS-30: Device: 100 cm H₂O; inlays 17.5 cm H₂O. Results stratified by aperture size (14 mm and 22 mm) were in line with all other findings with Device showing significant (p>0.001) advantages in burst pressure and failure rate.

A higher burst pressure observed for inlay grafts in open vs. constrained settings likely is due to the accuracy of positioning the inlay graft in the open setting, where the dura is trans-illuminated by the internal camera light and the graft can be placed more accurately directly below the incision, and the fibrin sealant provides some degree of stabilization (as the external clasp stabilizes the graft in Device). Restriction of view in the MIS setting negates the ability to precisely place the inlay graft, and even further diminished the inlay graft efficacy in a laminotomy model.

### Example 4

### In Vivo Animal Model Systems for Testing Tissue Repair and Sealing Devices

This Example provides in vivo model systems that may be adapted and employed for the testing various aspects of the tissue repair and sealing devices disclosed herein. Various physical properties and other parameters of tissue repair and sealing devices as disclosed herein may be tested in in vivo model systems, including in vivo model systems that are described in the scientific, medical, and patent literature and that may be configured for testing the repair and sealing of tissue fenestrations with the devices disclosed herein.

de Almeida, Otolaryngology Head Neck Surgery 141(2):184 (2009) and Seo, Journal of Clinical Neuroscience 58:187 (2018) describe in vivo porcine craniotomy model system that may be adapted for testing the repair of tissue fenestrations by assessing the leakage of cerebrospinal fluids (CSF). In de Almeida, pigs undergo a craniotomy to create fistula through the cribriform plate into the nasal cavity. CSF leaks may be assessed endoscopically prior to and following the repair of tissue fenestration. Inflammation and bone remodeling may be assessed via histopathological analysis.

Dafford, Spine Journal 15(5):1099 (2015) describes a comparison of the hydrostatic strength of dural repair techniques in a hydrostatic calf spine model system. Dural leakage is measured as a function of hydrostatic pressure and leak area. Leakage flow rate and the percent reduction of leak area is determined using analysis of variance (ANOVA).

Deng, Neurological Research 38(9):799 (2016); Preul, Neurosurgery 53(5):1189 (2003); and Zerris, Journal of Biomedical Materials Research 83(2):580 (2007) describe in vivo canine cranial dura and arachnoid model systems for assessing CSF leakage. Deng also reports macroscopic and microscopic observations at 30 and 90 days following dura repair. Preul reports the results of Valsalva tests at 1, 4, 7, and 56 days post-surgery and of histopathological analyses for control and treated animals.

Cosgrove, Journal of Neurosurgery 106:52 (2007); Osbun, World Neurosurgery 78(5):498 (2012); and Weinstein, Journal of Neurosurgery 112(2):219 (2010) describe in vivo craniotomy and craniectomy methodology that may be adapted for testing the repair of tissue fenestrations by assessing the leakage of CSF in humans. The neurological procedures used in Cosgrove are performed infratentorially or supratentorially using suboccipital, temporal, and frontal surgical approaches with durotomy lengths ranging from 1.0-19.0 cm. Osbun assesses complications resulting in unplanned postoperative interventions or reoperations following dural closure and compares the incidence of surgical site infections, CSF leaks, and other neurological complications in both treatment (dural repair) and control groups.

While heterologous bovine dura may be used for testing mechanical properties, tissue trauma, deployment efficacy and stability for the IM, bovine pericardium may be used as a dural membrane alternative due to its better consistency and physical characteristics closer to human dura. Experiments for sealing efficacy using bovine dura in the open pressure chamber model show comparable results to those seen with bovine pericardium, with the exception that the bovine was not a consistent or reliable waterproof membrane.

GLP-compliant biocompatibility and toxicology will establish safety. A rabbit craniotomy model will be used to characterize biocompatibility and long-term integrity of the graft-clasp assembly when applied through a durotomy. A non-GLP sheep laminectomy model will measure durability and function of the watertight seal and assess tissue reaction over 90 days. Additional biocompatibility studies will include cytotoxicity, sensitization, acute toxicity, genotoxicity, and carcinogenicity.

### Example 5

### An Ovine (Sheep) In Vivo Animal Model System for Testing Tissue Repair and Sealing Devices Under Constrained Conditions

This Example discloses the testing in vivo of GLP toxicology and long-term efficacy of tissue repair and sealing devices under constrained minimally invasive surgical (MIS) conditions in a clinically relevant ovine (male sheep) laminectomy model system.

Sheep were chosen due to the anatomic similarity of the lumbar spine and dura to that of humans. All pre-operative conditioning, surgery, immediate post-operative care and long-term care and monitoring were performed at an AAALAC-accredited facility (NAMSA) and protocols approved by the NAMSA IACUC. Test implants and the applicator/handpiece were manufactured, assembled, and sterilized in their final form. Sheep were acclimated in controlled humane conditions for 3-5 days prior to surgery. Following adequate anesthesia and preoperative antibiotics, a standard laminectomy was performed.

The tissue repair and sealing device was positioned via the 22 mm access port to apply and secure a 16 mm DuraMatrix Basic graft below the dura in treatment animals (N=6). In control animals, (N=6), a DuraMatrix Onlay Plus circular graft was advanced through the 22 mm port and placed beneath the dura in the usual fashion for inlay graft repair. Dural repairs in both groups were covered with Tisseel and artificial CSF instilled through the intrathecal catheter to bring the CSF pressure to 10 cm H₂O.

Observation of the dural repairs via video camera was continued for 30 minutes, during which 4 Valsalva maneuvers of 20 seconds each to intrathecal pressures of 40 cm H₂O were performed at 5 minute intervals, and repair sites closely observed for leakage, which was measured using the absorptive paper technique. The muscle, subcutaneous tissue and skin were reapproximated and sterile dressing applied to the wound. Results for time for closure and leak rate, timing and intrathecal burst pressure of the repair were recorded.

Post-operative outcome measures included (1) CSF leakage from the wound; (2) wound infection; and (3) anything necessitating euthanasia. At intervals of 4, 8, and 12 weeks, Control animals were euthanized for exploration of the surgical site to include analysis of the following outcome measures: (1) volume and infection of any paraspinal fluid collection; (2) ongoing CSF leak from the dura; (3) presence, appearance and weight of residual epidural polymer clasp material; and (4) signs and degree of gross inflammation in the dura and paraspinal muscles.

The dura at the laminectomy site was excised en bloc to the limits of the laminectomy and underwent histologic analysis, interpreted by a pathologist using a standard scale (1 to 4) to reflect the degree of response for each outcome; (1) physical status (volume, location) of the intradural graft and remaining polymer; (2) signs and degree of inflammation in dura and underlying nerves including characterization of white cell response (PMN, lymphocytes, Eosinophils); and (3) degree of fibroblast infiltration into graft and dural incision.

The ease of visualization, functional deployment, and precise positioning of the device were assessed through MIS laminotomy exposure. Animals underwent euthanasia and necropsy at designated time points for comprehensive post-mortem analysis, including visual inspection of the implant site, fluid collection, and culture, along with the excision and detailed examination of the implant and surrounding tissue.

A long-term (90-day) safety and efficacy study was performed in a sheep laminectomy model to assess the tissue repair and sealing device's biological integration and functional performance and to obtain data on local tissue response, long-term durability, and safety. Tissue repair and sealing devices consistently maintained a watertight seal for up to 90 days in ≥90% of grafts. No significant tissue reaction was observed. Optimal visualization, functional deployment, and positioning of a tissue repair and sealing device was achieved through MIS laminotomy exposure. Minimal impact on axonal integrity, gliosis, demyelination, and neuronal loss andinimal adhesion formation (i.e. scar tissue or unintended adhesions to dura or spinal cord) were observed. Complete dural healing was confirmed and a continuity of cellular infiltration was observed.

### Example 6

### A Bilateral Rabbit Craniotomy In Vivo Animal Model System for Testing Tissue Repair and Sealing Devices Under Constrained Conditions

This Example discloses the testing in vivo of biocompatibility and tissue reaction over a 90 day period in a bilateral rabbit craniotomy model. In four groups of five animals each, fully-grown male New Zealand White rabbits underwent bilateral 1.5 cm parietal craniotomies using a high-speed drill. Standardized 10 mm circular dural defects were created and repaired with a tissue repair and seal device and grafts were secured with a biodegradable clasp on one side, and a control inlay graft of commercially-available dural substitute material on the opposite side. Animals will be sacrificed at 2, 4, 8, and 12 weeks post-surgery and histologic assessment of biocompatibility performed according to GLP and ISO standards.

The entire dura exposed at the craniotomy will be circumferentially excised en bloc and histologic analysis performed by a pathologist to quantify using a standard scale the following responses; (1) inflammatory response (macrophage infiltration, lymphocytes, gliosis), (2) fibrosis and encapsulation, (3) angiogenesis, (4) tissue integration, (5) biodegradation profile and absorption rate, (6) neural tissue impact (e.g., astrocytosis, demyelination risk), (7) dural healing, (8) potential for adhesion formation, and (9) presence and volume of any extradural fluid collection. All preclinical safety studies will align with GLP standards (21 CFR Part 58), ISO 10993 (for Biocompatibility Testing), ISO 10993-6 (for Implantable Degradable Materials), and ASTM standards for bioresorbable implants. Comparative analysis with predicate devices are used to inspect intended uses, material compositions, degradation profiles, mechanical properties, and biocompatibility.

Additional biocompatibility testing was employed to assess cytotoxicity, sensitization, acute toxicity, genotoxicity, and carcinogenicity to verify the device's safety for human application. Rigorous testing will also evaluate degradation profile, sterility, shelf life, and packaging integrity. Standards from ISO 11137 (for radiation sterilization) and ISO 17665 (for steam sterilization) will guide the sterilization processes. Thorough validation of packaging integrity and sterility maintenance will comply with ASTM F1980 and ISO 11737-2.

### Example 7

### A Porcine Laminectomy In Vivo Animal Model System for Testing Tissue Repair and Sealing Devices Under Constrained Conditions

This Example discloses the testing in vivo of tissue repair and sealing devices in an acute pig laminectomy model. Device modifications were tested for (i) deployment of grafts of different sizes and shapes, (ii) fastening grafts for replacing dura in large dural defects, and (iii) enabling the capability to deliver adjunctive sealant.

Short-term device performance was evaluated in a non-survival, non-GLP proof-of-concept experiment in 4 male pigs. In the initial animal, the diameter of the thecal sac (12-14 mm) would not accommodate the circular 16 mm Device graft (DuraMatrix®) below the dura. The graft was trimmed into an oval shape and was successfully deployed with near complete cessation of CSF leakage. A lumbar drain was positioned at 30 cm above the heart, and observation of the dural repair via video camera was continued for 30 minutes, during which four Valsalva maneuvers of 20 seconds each to intrathoracic pressures of 20 cm H₂O were performed and CSF leak was quantitated by the paper towel absorption technique. The graft was removed and a 16 mm circular DuraMatrix Onlay Plus inlay graft placed beneath the dural incision and covered with the fibrin sealant

Tisseel, and CSF leak collected according to the same protocol. With the ability to rapidly modify Device components, PCS engineers laser cut 12 mm grafts and re-designed and printed the external clasp to work in the smaller implant; sealing efficacy for the smaller clasp was verified in vitro at PCS the following morning. The remaining 3 animals were then implanted with a device as disclosed herein vs inlay grafts through smaller durotomies (5 mm×7 mm). Both Device and the inlay graft reduced CSF leaks from a baseline dural incision CSF leak rate of 2.5-3 cc/min to less than 0.5 cc/min over the 30 minute monitoring period.

The scope of the disclosure is thus indicated by the appended claims rather than by the foregoing description, and all changes that come within meaning and range of equivalency of the claims are intended to be embraced herein.

What is claimed is:

1. A tissue repair and sealing device for use in an open or minimally invasive (MIS) surgical procedure to rapidly repair a tissue fenestration and create a pressure-resistant, watertight seal, said device comprising
(a) an applicator subassembly comprising, in operable connection, (i) a deployable graft-clasp lock mechanism, (ii) a deployable graft-clasp release mechanism, and (iii) a deployable graft-clasp positioning mechanism and
(b) a graft-clasp subassembly comprising, in operable connection, (i) a foldable graft, (ii) a deployable clasp, and (iii) a bioabsorbable coupler.

2. The tissue repair and sealing device of claim 1
wherein said graft-clasp lock mechanism comprises a first cylinder having a first diameter,
wherein said graft-clasp release mechanism comprises a second cylinder having a second diameter that is less than said first diameter,
wherein said graft-clasp positioning mechanism comprises a third cylinder having a third diameter that is less than said second diameter,
wherein said first, second, and third cylinders are aligned concentrically (i.e. nested), and
wherein each of said first, second, and third cylinders has a proximal end and a distal end.

3. The tissue repair and sealing device of claim 1 wherein deploying said graft-clasp positioning mechanism permits the positioning of a graft on a tissue inner surface and a clasp on a tissue outer surface.

4. The tissue repair and sealing device of claim 1 wherein deploying said graft-clasp lock mechanism locks a clasp into a graft-clasp coupler on said graft-clasp subassembly.

5. The tissue repair and sealing device of claim 1 wherein deploying said graft-clasp release mechanism disconnects the graft-clasp subassembly from the applicator subassembly at the distal end of said graft positioning cylinder.

6. The tissue repair and sealing device of claim 1 wherein said clasp comprises one or more: (1) expands [based on material characteristics] and (2) is water impermeable.

7. The tissue repair and sealing device of claim 1 wherein said clasp comprises one or more: (1) curved tips to facilitate strut sliding while locking, (2) folded toward distal end of the applicator subassembly, (3) a central ring with radial struts, and (4) inner flexible posts that pass over a coupler ball to lock onto coupler groove.

8. The tissue repair and sealing device of claim 1 wherein said coupler comprises one or more: (1) is not integrated with an external clasp and (2) comprises a locking groove for a corresponding clasp strut ring.

9. The tissue repair and sealing device of claim 1 wherein said applicator comprises one or more: (1) a stationary external constraining cylinder, (2) multiple bushings for graft and clasp to move within an external constraining cylinder, (3) an additional bushing to push ring onto locking groove, (4) a mechanism to disarticulate applicator from coupler, (5) a grip rocker trigger to actuate various bushings, (6) a grip thumb lever to disarticulate a central cylinder from a coupler ball, and (7) a grip cylinder for administration of sealant.

10. The tissue repair and sealing device of claim 1 wherein said deployable clasp assembly comprises one or more: (1) is configured to adopt a folded configuration when retained by said graft-clasp lock cylinder and to rapidly unfold to a pre-folded state.

11. The tissue repair and sealing device of claim 1 wherein said deployable clasp comprises a biopolymer selected from the group consisting of a polylactide (PLA), a polyglycolide (PGA), a polylactide-co-D, L lactide (PDLLA), a polylactide-co-glycolide (PLGA), a polylactide-co-caprolactone (PLCL), a polycaprolactone (PCL), a polydioxanone (PDO), and a polylactide-co-trimethylene carbonate (PL-TMC),
wherein said biopolymer exhibits shape memory and superelasticity characteristics that permit the folding of said biopolymer while retaining the capacity to rapidly unfold to a pre-folded state.

12. The tissue repair and sealing device of claim 1 wherein said deployable clasp assembly comprises a biocompatible, non-ferromagnetic, passivated metal or metal alloy wire that is selected from the group consisting of pure titanium; a titanium-based alloy; a cobalt-based alloy; a platinum-based alloy; and a molybdenum, tungsten, and tantalum alloy.
wherein said biocompatible, non-ferromagnetic, passivated metal or metal alloy wire exhibits shape memory and superelasticity characteristics that permit the folding of said wire while retaining the capacity to rapidly unfold to a pre-folded state.

13. The tissue repair and sealing device of claim 12 wherein said biocompatible, non-ferromagnetic, passivated metal or metal alloy is selected from the group consisting of a nickel-titanium alloy (Nitinol) and a niobium-titanium alloy.

14. The tissue repair and sealing device of claim 1 wherein said graft assembly is configured (a) to adopt a folded configuration when traversing a tissue fenestration or when retained by said graft-clasp lock cylinder and (b) to rapidly unfold to a pre-folded state.

15. The tissue repair and sealing device of claim 1 wherein said graft is selected from the group consisting of an autograft, an isograft, an allograft, and a xenograft and
wherein said graft is derived from an animal tissue is selected from the group consisting a human tissue, a bovine tissue, and a porcine tissue.

16. The tissue repair and sealing device of claim 1 wherein said graft material comprises one or more synthetic material selected from the group consisting of poly(ethylene terephthalate) and expanded polytetrafluoroethylene (ePTF).

17. The tissue repair and sealing device of claim 1 wherein said graft comprises an acellular, porous extracellular matrix scaffold of collagen, elastin, and, optionally, a growth factor.

18. The tissue repair and sealing device of claim 1 wherein said graft comprises a dural substitute selected from the group consisting of Duraform® dural graft implant, Biodesign® Dural Graft, DuraGen® Matrix, DuraMatrix® dural graft, Cerafix dural Graft®, PRECLUDE®, Lyoplant Onlay Graft®, Neuro-Patch Dural Graft®, SEAMDURA®, and Durepair™ Regeneration Matrix.

19. The tissue repair and sealing device of claim 1 wherein said graft comprises a drug eluting matrix.

20. The tissue repair and sealing device of claim 1 wherein said graft comprises a biocompatible, non-ferromagnetic, passivated metal or metal alloy wire that is selected from the group consisting of pure titanium; a titanium-based alloy; a cobalt-based alloy; a platinum-based alloy; and a molybdenum, tungsten, and tantalum alloy.
wherein said biocompatible, non-ferromagnetic, passivated metal or metal alloy wire exhibits shape memory and superelasticity characteristics that permit the folding of said wire while retaining the capacity to rapidly unfold to a pre-folded state.

21. The tissue repair and sealing device of claim 13 wherein said biocompatible, non-ferromagnetic, passivated metal or metal alloy is selected from the group consisting of a nickel-titanium alloy (Nitinol) and a niobium-titanium alloy.
